SEQUENCE LISTING (1 sequence)

SEQ ID NO: 1 - protein, 14 aa, Artificial Sequence
  Biotinylated Synthetic peptide
ARKRERAYSFGHHA

(19) United States
(12) Patent Application Publication
(10) Pub. No.: US 2008/0255143 A1
(43) Pub. Date: Oct. 16, 2008

(54) Inhibitors of Akt Activity

(75) Inventors: Dirk A. Heerding, Collegeville, PA (US); Tammy J. Clark, Collegeville, PA (US); David Harold Drewry, Resarch Triangle Park, NC (US); Jack Dale Leber, Collegeville, PA (US); Igor Safonov, Collegeville, PA (US); Dennis S. Yamashita, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia (PA)

(21) Appl. No.: 10/565,329

(22) PCT Filed: Jul. 28, 2004
(86) PCT No.: PCT/US04/24340

§ 371 Date: Oct. 26, 2007

Related U.S. Application Data
(60) Provisional application No. 60/490,851, filed on Jul. 29, 2003.
(60) Provisional application No. 60/491,055, filed on Jul. 30, 2003.
(60) Provisional application No. 60/493,101, filed on Aug. 6, 2003.
(60) Provisional application No. 60/494,752, filed on Aug. 13, 2003.
(60) Provisional application No. 60/507,014, filed on Sep. 29, 2003.
(60) Provisional application No. 60/530,847, filed on Dec. 18, 2003.

(51) Int. Cl. A61K 31/437; C07D 471/04; A61P 35/00; A61K 31/496

(52) U.S. Cl. 514/253.04; 546/118; 544/362; 514/303

(57) ABSTRACT

Invented are novel 1H-imidazo[4,5-c]pyridin-2-yl compounds, the use of such compounds as inhibitors of protein kinase B activity and in the treatment of cancer and arthritis.

### FIELD OF THE INVENTION

This invention relates to novel 1H-imidazo[4,5-c]pyridin-2-yl compounds, the use of such compounds as inhibitors of protein kinase B (hereinafter PKB/Akt, PKB or Akt) activity and in the treatment of cancer and arthritis.

### BACKGROUND OF THE INVENTION

The present invention relates to 1H-imidazo[4,5-c]pyridin-2-yl containing compounds that are inhibitors of the activity of one or more of the isoforms of the serine/threonine kinase, Akt (also known as protein kinase B). The present invention also relates to pharmaceutical compositions comprising such compounds and methods of using the instant compounds in the treatment of cancer and arthritis (Liu et al. Current Opin. Pharmacology 3:317-22 (2003)).

Apoptosis (programmed cell death) plays essential roles in embryonic development and pathogenesis of various diseases, such as degenerative neuronal diseases, cardiovascular diseases and cancer. Recent work has led to the identification of various pro- and anti-apoptotic gene products that are involved in the regulation or execution of programmed cell death. Expression of anti-apoptotic genes, such as Bcl2 or Bcl-x_{L}, inhibits apoptotic cell death induced by various stimuli. On the other hand, expression of pro-apoptotic genes, such as Bax or Bad, leads to programmed cell death (Adams et al. Science, 281:1322-1326 (1998)). The execution of programmed cell death is mediated by caspase-1 related proteinases, including caspase-3, caspase-7, caspase-8 and caspase-9 etc (Thornberry et al. Science, 281:1312-1316 (1998)).

The phosphatidylinositol 3′-OH kinase (PI3K)/Akt/PKB pathway appears important for regulating cell survival/cell death (Kulik et al. Mol. Cell. Biol. 17:1595-1606 (1997); Franke et al, Cell, 88:435-437 (1997); Kauffmann-Zeh et al. Nature 385:544-548 (1997) Hemmings Science, 275:628-630 (1997); Dudek et al., Science, 275:661-665 (1997)). Survival factors, such as platelet derived growth factor (PDGF), nerve growth factor (NGF) and insulin-like growth factor-1 (IGF-1), promote cell survival under various conditions by inducing the activity of PI3K (Kulik et al. 1997, Hemmings 1997). Activated PI3K leads to the production of phosphatidylinositol (3,4,5)-triphosphate (PtdIns (3,4,5)—P3), which in turn binds to, and promotes the activation of, the serine/threonine kinase Akt, which contains a pleckstrin homology (PH)-domain (Franke et al Cell, 81:727-736 (1995); Hemmings Science, 277:534 (1997); Downward, Curr. Opin. Cell Biol. 10:262-267 (1998), Alessi et al., EMBO J. 15: 6541-6551 (1996)). Specific inhibitors of PI3K or dominant negative Akt/PKB mutants abolish survival-promoting activities of these growth factors or cytokines. It has been previously disclosed that inhibitors of PI3K (LY294002 or wortmannin) blocked the activation of Akt/PKB by upstream kinases. In addition, introduction of constitutively active PI3K or Akt/PKB mutants promotes cell survival under conditions in which cells normally undergo apoptotic cell death (Kulik et al. 1997, Dudek et al. 1997).

Analysis of Akt levels in human tumors showed that Akt2 is overexpressed in a significant number of ovarian (J. Q. Cheung et al. Proc. Natl. Acad. Sci. U.S.A. 89:9267-9271 (1992)) and pancreatic cancers (J. Q. Cheung et al. Proc. Natl. Acad. Sci. U.S.A. 93:3636-3641 (1996)). Similarly, Akt3 was found to be overexpressed in breast and prostate cancer cell lines (Nakatani et al. J. Biol. Chem. 274:21528-21532 (1999). It was demonstrated that Akt-2 was over-expressed in 12% of ovarian carcinomas and that amplification of Akt was especially frequent in 50% of undifferentiated tumors, suggestion that Akt may also be associated with tumor aggressiveness (Bellacosa, et al, Int J. Cancer, 64, pp. 280-285, 1995). Increased Akt1 kinase activity has been reported in breast, ovarian and prostate cancers (Sun et al. Am. J. Pathol. 159:431-7 (2001)).

The tumor suppressor PTEN, a protein and lipid phosphatase that specifically removes the 3′ phosphate of PtdIns(3,4,5)-P3, is a negative regulator of the PI3K/Akt pathway (Li et al. Science 275:1943-1947 (1997), Stambolic et al. Cell 95:29-39 (1998), Sun et al. Proc. Natl. Acad. Sci. U.S.A. 96:6199-6204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-3663 (1997), Risinger et al. Cancer Research 57:4736-4738 (1997)).

These observations demonstrate that the PI3K/Akt pathway plays important roles for regulating cell survival or apoptosis in tumorigenesis.

Three members of the Akt/PKB subfamily of second-messenger regulated serine/threonine protein kinases have been identified and termed Akt1/PKBα, Akt2/PKBβ, and Akt3/PKBγ respectively. The isoforms are homologous, particularly in regions encoding the catalytic domains. Akt/PKBs are activated by phosphorylation events occurring in response to PI3K signaling. PI3K phosphorylates membrane inositol phospholipids, generating the second messengers phosphatidyl-inositol 3,4,5-trisphosphate and phosphatidylinositol 3,4-bisphosphate, which have been shown to bind to the PH domain of Akt/PKB. The current model of Akt/PKB activation proposes recruitment of the enzyme to the membrane by 3′-phosphorylated phosphoinositides, where phosphorylation of the regulatory sites of Akt/PKB by the upstream kinases occurs (B. A. Hemmings, Science 275:628-630 (1997); B. A. Hemmings, Science 276:534 (1997); J. Downward, Science 279:673-674 (1998)).

Phosphorylation of Akt1/PKBα occurs on two regulatory sites, Thr³⁰⁸ in the catalytic domain activation loop and on Ser⁴⁷³ near the carboxy terminus (D. R. Alessi et al. EMBO J. 15:6541-6551 (1996) and R. Meier et al. J. Biol. Chem. 272:30491-30497 (1997)). Equivalent regulatory phosphorylation sites occur in Akt2/PKBβ and Akt3/PKBγ. The upstream kinase, which phosphorylates Akt/PKB at the activation loop site has been cloned and termed 3′-phosphoinositide dependent protein kinase 1 (PDK1). PDK1 phosphorylates not only Akt/PKB, but also p70 ribosomal S6 kinase, p90RSK, serum and glucocorticoid-regulated kinase (SGK), and protein kinase C. The upstream kinase phosphorylating the regulatory site of Akt/PKB near the carboxy terminus has not been identified yet, but recent reports imply a role for the integrin-linked kinase (ILK-1), a serine/threonine protein kinase, or autophosphorylation.

Inhibition of Akt activation and activity can be achieved by inhibiting PI3K with inhibitors such as LY294002 and wortmannin. However, PI3K inhibition has the potential to indiscriminately affect not just all three Akt isozymes but also other PH domain-containing signaling molecules that are dependent on PdtIns(3,4,5)-P3, such as the Tec family of tyrosine kinases. Furthermore, it has been disclosed that Akt can be activated by growth signals that are independent of PI3K.

Alternatively, Akt activity can be inhibited by blocking the activity of the upstream kinase PDK1. The compound UCN-01 is a reported inhibitor of PDK1. Biochem. J. 375(2):255 (2003). Again, inhibition of PDK1 would result in inhibition of multiple protein kinases whose activities depend on PDK1, such as atypical PKC isoforms, SGK, and S6 kinases (Williams et al. Curr. Biol. 10:439-448 (2000).

Small molecule inhibitors of Akt are useful in the treatment of tumors, especially those with activated Akt (e.g. PTEN null tumors and tumors with ras mutations). PTEN is a critical negative regulator of Akt and its function is lost in many cancers, including breast and prostate carcinomas, glioblastomas, and several cancer syndromes including Bannayan-Zonana syndrome (Maehama, T. et al. Annual Review of Biochemistry, 70: 247 (2001)), Cowden disease (Parsons, R.; Simpson, L. Methods in Molecular Biology (Totowa, N.J., United States), 222 (Tumor Suppressor Genes, Volume 1): 147 (2003)), and Lhermitte-Duclos disease (Backman, S. et al. Current Opinion in Neurobiology, 12(5): 516 (2002)). Akt3 is up-regulated in estrogen receptor-deficient breast cancers and androgen-independent prostate cancer cell lines and Akt2 is over-expressed in pancreatic and ovarian carcinomas. Akt1 is amplified in gastric cancers (Staal, Proc. Natl. Acad. Sci. USA 84: 5034-7 (1987) and upregulated in breast cancers (Stal et al. Breast Cancer Res. 5: R37-R44 (2003)). Therefore a small molecule Akt inhibitor is expected to be useful for the treatment of these types of cancer as well as other types of cancer. Akt inhibitors are also useful in combination with further chemotherapeutic agents.

It is an object of the instant invention to provide novel compounds that are inhibitors of Akt/PKB.

It is also an object of the present invention to provide pharmaceutical compositions that comprise a pharmaceutical carrier and compounds useful in the methods of the invention.

It is also an object of the present invention to provide a method for treating cancer that comprises administering such inhibitors of Akt/PKB activity.

It is also an object of the present invention to provide a method for treating arthritis that comprises administering such inhibitors of Akt/PKB activity.

### SUMMARY OF THE INVENTION

This invention relates to novel compounds of Formula (I):

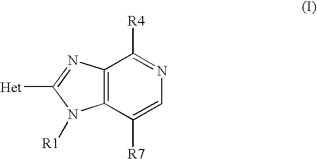

wherein:

Het is selected from the group consisting of:

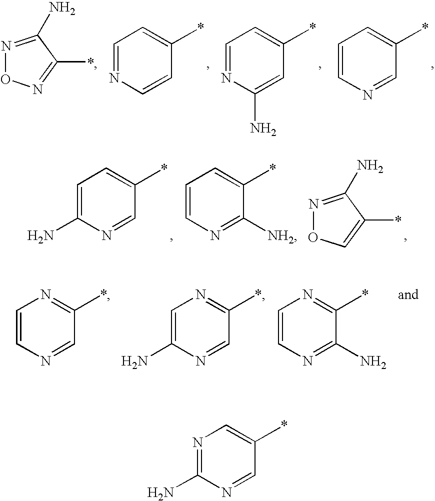

R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 4 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶, —S(O)ₙR² and protected —OH, where n is 0-2,
   R² is selected from hydrogen, alkyl, cycloalkyl, C₁-C₁₂aryl, substituted alkyl, substituted cycloalkyl and substituted C₁-C₁₂aryl, and
   R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from hydrogen, —C(O)NR⁹R¹⁰, —(CH₂)ₙNR⁹R¹⁰, —SO₂NR⁹R¹⁰, —(CH₂)ₙOR⁸, —O—(CH₂)ₘNR⁹R¹⁰ and —N—(CH₂)ₘNR⁹R¹⁰,
   where n is 0-2,
   m is 1-6, where the carbon chain formed by m is optionally substituted,
   R⁸ is alkyl, cycloalkyl, cycloalkyl containing from 1 to 4 heteroatoms, and aryl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 4 heteroatoms, substituted cycloalkyl containing from 1 to 4 heteroatoms, substituted cycloalkyl, halogen, aryl, substituted aryl and protected-OH,
      where R² and R³ are independently hydrogen, alkyl, cycloalkyl, C₁-C₁₂aryl, substituted alkyl, substituted cycloalkyl and substituted C₁-C₁₂aryl, and n is 0-2,
   R⁹ and R¹⁰ are independently hydrogen, cycloalkyl, cycloalkyl containing from 1 to 4 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 4 heteroatoms, substituted cycloalkyl, halogen, aryl, substituted aryl and protected —OH,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

This invention relates to a method of treating cancer, which comprises administering to a subject in need thereof an effective amount of an Akt/PKB inhibiting compound of Formula (I).

This invention relates to a method of treating arthritis, which comprises administering to a subject in need thereof an effective amount of an Akt/PKB inhibiting compound of Formula (I).

The present invention also relates to the discovery that the compounds of Formula (I) are active as inhibitors of Akt/PKB.

In a further aspect of the invention there is provided novel processes and novel intermediates useful in preparing the presently invented Akt/PKB inhibiting compounds.

Included in the present invention are pharmaceutical compositions that comprise a pharmaceutical carrier and compounds useful in the methods of the invention.

Also included in the present invention are methods of co-administering the presently invented Akt/PKB inhibiting compounds with further active ingredients.

### DETAILED DESCRIPTION OF THE INVENTION

This invention relates to compounds of Formula (I) as described above.

The presently invented compounds of Formula (I) inhibit Akt/PKB activity. In particular, the compounds disclosed herein inhibit each of the three Akt/PKB isoforms.

Included among the presently invented compounds of Formula (I) are those having Formula (II):

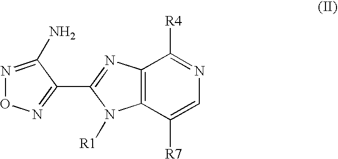

wherein:
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 4 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶, —S(O)ₙR² and protected —OH,
   where n is 0-2,
   R² is selected from hydrogen, alkyl, cycloalkyl, C₁-C₁₂aryl, substituted alkyl, substituted cycloalkyl and substituted C₁-C₁₂aryl, and
   R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from hydrogen, —C(O)NR⁹R¹⁰, —(CH₂)ₙNR⁹R¹⁰, —SO₂NR⁹R¹⁰, —(CH₂)ₙOR⁸, —O—(CH₂)ₘNR⁹R¹⁰ and —N—(CH₂)ₘNR⁹R¹⁰,
   where n is 0-2,
   m is 1-6, where the carbon chain formed by m is optionally substituted,
   R⁸ is alkyl, cycloalkyl, cycloalkyl containing from 1 to 4 heteroatoms, and aryl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 4 heteroatoms, substituted cycloalkyl containing from 1 to 4 heteroatoms, substituted cycloalkyl, halogen, aryl, substituted aryl and protected —OH,
      where R² and R³ are independently hydrogen, alkyl, cycloalkyl, C₁-C₁₂aryl, substituted alkyl, substituted cycloalkyl and substituted C₁-C₁₂aryl, and n is 0-2,
   R⁹ and R¹⁰ are independently hydrogen, cycloalkyl, cycloalkyl containing from 1 to 4 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 4 heteroatoms, substituted cycloalkyl, halogen, aryl, substituted aryl and protected —OH,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (I) are those having Formula (III):

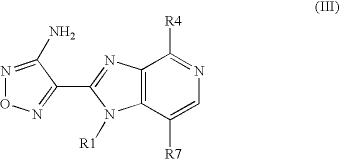

wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶, —S(O)ₙR² and protected —OH,
   where n is 0-2,
   R² is selected from hydrogen, alkyl, cycloalkyl, C₁-C₁₂aryl, substituted alkyl, substituted cycloalkyl and substituted C₁-C₁₂aryl, and
   R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from —C(O)NR⁹R¹⁰, —(CH₂)ₙNR⁹R¹⁰, —SO₂NR⁹R¹⁰, (CH₂)ₙOR⁸, —O—(CH₂)ₘNR⁹R¹⁰ and —N—(CH₂)ₘNR⁹R¹⁰,
   where n is 0-2,
   m is 1-6, where the carbon chain formed by m is optionally substituted,
   R⁸ is alkyl, piperidine, imidazolidine, phenyl, piperazine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 3 heteroatoms, substituted cycloalkyl containing from 1 to 3 heteroatoms, substituted cycloalkyl, halogen, aryl, substituted aryl and protected —OH,
      where R² and R³ are independently hydrogen, alkyl, cycloalkyl, C₁-C₁₂aryl, substituted alkyl, substituted cycloalkyl and substituted C₁-C₁₂aryl, and n is 0-2,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl, substituted aryl and protected —OH,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (I) are those having Formula (IV):

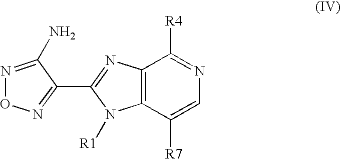

wherein:
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR², —C(O)NR⁵R⁶, —S(O)₂NR⁵R⁶, —S(O)ₙR² and protected —OH, where n is 0-2,
   R² is selected from hydrogen, alkyl, cycloalkyl, C₁-C₁₂aryl, substituted alkyl, substituted cycloalkyl and substituted C₁-C₁₂aryl, and
   R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is hydrogen;
   and/or pharmaceutically acceptable salts, hydrates, solvates and pro-drugs thereof.

Included among the presently invented compounds of Formula (II) are those in which:
R¹ is selected from: alkyl, alkyl substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen; and
R⁷ is selected from hydrogen, —C(O)NR⁹R¹⁰ and —(CH₂)ₙOR⁸, where n is 0-2;
   R⁸ is alkyl, piperidine, imidazolidine, piperidyl and pyrrolidinyl, each of which is optionally substituted with one or more substituents selected from the group consisting of: alkoxy, acyloxy, aryloxy, amino, N-acylamino, hydroxy, nitro, cyano, cycloalkyl, halogen and C₁-C₁₂aryl,
   R⁹ and R¹⁰ are independently hydrogen, cycloalkyl, cycloalkyl containing from 1 to 3′heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (III) are those in which:
R¹ is selected from: alkyl, alkyl substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen; and
R⁷ is selected from —C(O)NR⁹R¹⁰ and —(CH₂)ₙOR⁸, where n is 0-2;
   R⁸ is alkyl, piperidine, imidazolidine, piperidyl and pyrrolidinyl, each of which is optionally substituted with one or more substituents selected from the group consisting of: alkoxy, acyloxy, aryloxy, amino, N-acylamino, hydroxy, nitro, cyano, cycloalkyl, halogen and C₁-C₁₂aryl,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (IV) are those in which:
R¹ is selected from: alkyl, alkyl substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen; and
R⁷ is hydrogen;
   and/or pharmaceutically acceptable salts, hydrates, solvates and pro-drugs thereof.

Included among the presently invented compounds of Formula (II) are those in which:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from, —C(O)NR⁹R¹⁰, —(CH₂)ₙNR⁹R¹⁰, —(CH₂)ₙOR⁸, —O—(CH₂)ₘNR⁹R¹⁰ and —N—(CH₂)ₘNR⁹R¹⁰,
   where n is 0-2;
   m is 1-6, where the carbon chain formed by m is optionally substituted,
   R⁸ is alkyl, piperidine, imidazolidine, phenyl, piperazine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, hydroxy, nitro, guanadine, substituted guanadine, cyano, cycloalkyl, substituted cycloalkyl, cycloalkyl containing from 1 to 3 heteroatoms, substituted cycloalkyl containing from 1 to 3 heteroatoms, halogen, C₁-C₁₂aryl and substituted C₁-C₁₂aryl,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl;
      except 4-[1-Ethyl-7-(piperidin-4-yloxy)-1H-imidazo[4,5-c]pyridin-2-yl]-furazan-3-ylamine
         and/or pharmaceutically acceptable salts, hydrates, solvates and pro-drugs thereof.

Included among the presently invented compounds of Formula (III) are those in which:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro, and halogen; and
R⁷ is selected from —(CH₂)ₙOR⁸, —O—(CH₂)ₘNR⁹R¹⁰ and —N—(CH₂)ₘNR⁹R¹⁰,
   where n is 0-2;
   m is 1-6, where the carbon chain formed by m is optionally substituted,
   R⁸ is alkyl, piperidine, imidazolidine, phenyl, piperazine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, hydroxy, nitro, guanadine, substituted guanadine, cyano, cycloalkyl, substituted cycloalkyl, cycloalkyl containing from 1 to 3 heteroatoms, substituted cycloalkyl containing from 1 to 3 heteroatoms, halogen, C₁-C₁₂aryl and substituted C₁-C₁₂aryl,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,
   or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (IV) are those in which:
R¹ is selected from: alkyl, alkyl substituted with one or more substituents selected from the group consisting of: hydroxy, alkoxy, amino, N-acylamino, cyclopropyl and halogen, cylcoalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro, cyano and halogen; and
R⁷ is hydrogen;
   and/or pharmaceutically acceptable salts, hydrates, solvates and pro-drugs thereof.

Included among the presently invented compounds of Formula (II) are those in which:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from alkyl, alkyl substituted with one or more substituents selected from the group consisting of: hydroxy, methoxy, ethoxy, amino, N-acylamino, cyclopropyl and halogen, cycloalkyl, cycloalkyl containing from 1 to 3 heteroatoms and C₁-C₁₂aryl; and
R⁷ is selected from —(CH₂)ₙNR⁹R¹⁰, —(CH₂)ₙOR⁸, —O—(CH₂)ₘNR⁹R¹⁰ and —N—(CH₂)ₘNR⁹R¹⁰,
   where n is 0-2;
   m is 1-6, where the carbon chain formed by m is optionally substituted,
   R⁸ is alkyl, piperidine, piperidyl and pyrrolidinyl, each of which is optionally substituted with one or more substituents selected from the group consisting of: methoxy, ethoxy, acyloxy, aryloxy, amino, amino substituted with one or more substituents selected from the group consisting of: hydroxy, alkoxy and amino, N-acylamino, hydroxy, nitro, guanadine, substituted guanadine, cyano, cycloalkyl, substituted cycloalkyl, cycloalkyl containing from 1 to 3 heteroatoms, substituted cycloalkyl containing from 1 to 3 heteroatoms, halogen, C₁-C₁₂aryl and substituted C₁-C₁₂aryl,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, nitro, cyano, cycloalkyl, halogen, aryl and substituted aryl;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (III) are those in which:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from alkyl, alkyl substituted with one or more substituents selected from the group consisting of: hydroxy, methoxy, ethoxy, amino, cyclopropyl and halogen, cycloalkyl, cycloalkyl containing from 1 to 3 heteroatoms and C₁-C₁₂aryl; and
R⁷ is selected from —(CH₂)ₙNR⁹R¹⁰, —(CH₂)ₙOR⁸, —O—(CH₂)ₘNR⁹R¹⁰ and —N—(CH₂)ₘNR⁹R¹⁰,
   where n is 0-2;
   m is 1-6, where the carbon chain formed by m is optionally substituted,
   R⁸ is alkyl, piperidine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, hydroxy, nitro, guanadine, substituted guanadine, cyano, cycloalkyl, substituted cycloalkyl, cycloalkyl containing from 1 to 3 heteroatoms, substituted cycloalkyl containing from 1 to 3 heteroatoms, halogen, C₁-C₁₂aryl and substituted C₁-C₁₂aryl,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, aryloxy, amino, oxo, hydroxy, methylamino, dimethylamino, hydroxyalkyl, nitro, cyano, cycloalkyl, halogen, C₁-C₁₂aryl and substituted C₁-C₁₂aryl;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (IV) are those in which:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from alkyl, alkyl substituted with one or more substituents selected from the group consisting of: hydroxy, methoxy, ethoxy, amino, cyclopropyl and halogen, cycloalkyl, cycloalkyl containing from 1 to 3 heteroatoms and C₁-C₁₂aryl; and
R⁷ is hydrogen;
   and/or pharmaceutically acceptable salts, hydrates, solvates and pro-drugs thereof.

Included among the presently invented compounds of Formula (I) are those in which:
R¹ is selected from alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶ and —S(O)ₙR²,
   where n is 0-2,
   R² is selected from hydrogen, alkyl, cycloalkyl, C₁-C₁₂aryl, substituted alkyl, substituted cycloalkyl and substituted C₁-C₁₂aryl, and
   R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl and substituted aryl,
   or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from —(CH₂)ₙOR⁸, —O—(CH₂)ₘNR⁹R¹⁰ and —N—(CH₂)ₘNR⁹R¹⁰,
   where n is 0-2,
   m is 1-6, where the carbon chain formed by m is optionally substituted,
   R⁸ is alkyl substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 3 heteroatoms, substituted cycloalkyl, substituted cycloalkyl containing from 1 to 3 heteroatoms, halogen, aryl and substituted aryl, cycloalkyl and cycloalkyl containing from 1 to 3 heteroatoms, each of said cycloalkyl and cycloalkyl containing from 1 to 3 heteroatoms is optionally substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(b)₂NR²R³, nitro, cyano, cycloalkyl, cycloalkyl containing from 1 to 3 heteroatoms, substituted cycloalkyl, halogen, aryl and substituted aryl,
      where R² and R³ are independently hydrogen, alkyl, cycloalkyl, C₁-C₁₂aryl, substituted alkyl, substituted cycloalkyl and substituted C₁-C₁₂aryl, and n is 0-2,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
      where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (II) are those in which:
R¹ is selected from alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶ and —S(O)ₙR²,
   where n is 0-2,
   R² is selected from hydrogen, alkyl, cycloalkyl, C₁-C₁₂aryl, substituted alkyl, substituted cycloalkyl and substituted C₁-C₁₂aryl, and
   R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl and substituted aryl, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
      where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from —(CH₂)ₙOR⁸, —O—(CH₂)ₘNR⁹R¹⁰ and —N—(CH₂)ₘNR⁹R¹⁰,
   where n is 0-2,
   m is 1-6, where the carbon chain formed by m is optionally substituted,
   R⁸ is alkyl substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 3 heteroatoms, substituted cycloalkyl, substituted cycloalkyl containing from 1 to 3 heteroatoms, halogen, aryl and substituted aryl, cycloalkyl and cycloalkyl containing from 1 to 3 heteroatoms, each of said cycloalkyl and cycloalkyl containing from 1 to 3 heteroatoms is optionally substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, cycloalkyl containing from 1 to 3 heteroatoms, substituted cycloalkyl, halogen, aryl and substituted aryl,
      where R² and R³ are independently hydrogen, alkyl, cycloalkyl, C₁-C₁₂aryl, substituted alkyl, substituted cycloalkyl and substituted C₁-C₁₂aryl, and n is 0-2,
   R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
      where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine
   and/or pharmaceutically acceptable salts, hydrates, solvates and pro-drugs thereof.

Included among the presently invented compounds of Formula (I) are those in which:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
   where n is 0-2,
   m is 1-6, where the carbon chain formed by m is optionally substituted,
   R⁸ is alkyl substituted with one or more substituents selected from the group consisting of: cycloalkyl, cycloalkyl containing from 1 to 3 heteroatoms, substituted cycloalkyl, substituted cycloalkyl containing from 1 to 3 heteroatoms, aryl and substituted aryl,
   R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
      where R² and R³ are independently hydrogen, alkyl, cycloalkyl, C₁-C₁₂aryl, substituted alkyl, substituted cycloalkyl and substituted C₁-C₁₂aryl, and n is 0-2;
         and/or pharmaceutically acceptable salts, hydrates, solvates and pro-drugs thereof.

Included among the presently invented compounds of Formula (II) are those in which:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
   where n is 0-2,
   m is 1-6, where the carbon chain formed by m is optionally substituted,
   R⁸ is alkyl substituted with one or more substituents selected from the group consisting of: cycloalkyl, cycloalkyl containing from 1 to 3 heteroatoms, substituted cycloalkyl, substituted cycloalkyl containing from 1 to 3 heteroatoms, aryl and substituted aryl,
   R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
      where R² and R³ are independently hydrogen, alkyl, cycloalkyl, C₁-C₁₂aryl, substituted alkyl, substituted cycloalkyl and substituted C₁-C₁₂aryl, and n is 0-2;
         and/or pharmaceutically acceptable salts, hydrates, solvates and pro-drugs thereof.

Included among the presently invented compounds of Formula (I) are those in which:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
   where n is 0-2,
   m is 1-6, where the carbon chain formed by m is optionally substituted,
   R⁸ is alkyl substituted with one or more substituents selected from the group consisting of: piperidine, substituted piperidine, phenyl and, substituted phenyl,
   R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, nitro, cyano, cycloalkyl, cycloalkyl containing from 1 to 3 heteroatoms, halogen and aryl;
      and/or pharmaceutically acceptable salts, hydrates, solvates and pro-drugs thereof.

Included among the presently invented compounds of Formula (II) are those in which:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
   where n is 0-2,
   m is 1-6, where the carbon chain formed by m is optionally substituted,
   R⁸ is alkyl substituted with one or more substituents selected from the group consisting of: piperidine, substituted piperidine, phenyl and, substituted phenyl,
   R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, nitro, cyano, cycloalkyl, cycloalkyl containing from 1 to 3 heteroatoms, halogen and aryl;
      and/or pharmaceutically acceptable salts, hydrates, solvates and pro-drugs thereof.

Included among the novel compounds useful in the present invention are:
4-(4-Phenyl-1-piperidin-4-yl-1H-imidazo[4,5-c]pyridin-2-yl)-furazan-3-ylamine;
4-[4-(3-Chloro-phenyl)-1-piperidin-4-yl-1H-imidazo-[4,5-c]pyridin-2-yl]furazan-3-ylamine;
4-[1-(3-Amino-2,2-dimethylpropyl)-4-(3-chlorophenyl)-1H-imidazo[4,5-c]pyridin-2-yl]-furazan-3-ylamine;
4-[1-(cyclopropylmethyl)-4-(2-methylphenyl)-1H-imidazo[4,5-c]pyridin-2-yl]-1,2,5-oxadiazol-3-amine;
4-[4-(2-chlorophenyl)-1-(cyclopropylmethyl)-1H-imidazo[4,5-c]pyridin-2-yl]-1,2,5-oxadiazol-3-amine;
4-[1-(3-Amino-2,2-dimethylpropyl)-4-phenyl-1H-imidazo[4,5-c]pyridinyl-2-yl]-furazan-3-ylamine;
4-[4-(3-chlorophenyl)-1-(cyclopropylmethyl)-1H-imidazo[4,5-c]pyridin-2-yl]-1,2,5-oxadiazol-3-amine;
4-[4-chloro-1-(cyclopropylmethyl)-1H-imidazo[4,5-c]pyridin-2-yl]-1,2,5-oxadiazol-3-amine;
4-[1-(cyclopropylmethyl)-4-(3-furanyl)-1H-imidazo[4,5-c]pyridin-2-yl]-1,2,5-oxadiazol-3-amine;
4-[1-(5-aminopentyl)-4-phenyl-1H-imidazo[4,5-c]pyridin-2-yl]-1,2,5-oxadiazol-3-amine;
4-[1-(6-aminohexyl)-4-phenyl-1H-imidazo[4,5-c]pyridin-2-yl]-1,2,5-oxadiazol-3-amine;
4-[1-(5-aminopentyl)-4-(3-chlorophenyl)-1H-imidazo[4,5-c]pyridin-2-yl]-1,2,5-oxadiazol-3-amine;
4-[1-(6-aminohexyl)-4-(3-chlorophenyl)-1H-imidazo[4,5-c]pyridin-2-yl]-1,2,5-oxadiazol-3-amine;
4-[1-(3-Amino-2,2-dimethylpropyl)-4-(3-methoxyphenyl)-1H-imidazo[4,5-c]pyridinyl-2-yl]-furazan-3-ylamine;
4-[1-(5-aminopentyl)-4-(3-thienyl)-1H-imidazo[4,5-c]pyridin-2-yl]-1,2,5-oxadiazol-3-amine;
4-[1-(6-aminohexyl)-4-(3-thienyl)-1H-imidazo[4,5-c]pyridin-2-yl]-1,2,5-oxadiazol-3-amine;
4-[4-phenyl-1-(3-piperidinylmethyl)-1H-imidazo[4,5-c]pyridin-2-yl]-1,2,5-oxadiazol-3-amine;
4-[4-(3-chlorophenyl)-1-(3-piperidinylmethyl)-1H-imidazo[4,5-c]pyridin-2-yl]-1,2,5-oxadiazol-3-amine;
4-[4-(4-chlorophenyl)-1-(3-piperidinylmethyl)-1H-imidazo[4,5-c]pyridin-2-yl]-1,2,5-oxadiazol-3-amine;
4-[1-(3-aminopropyl)-4-(2-thienyl)-1H-imidazo[4,5-c]pyridin-2-yl]-1,2,5-oxadiazol-3-amine;
4-[1-(3-aminopropyl)-4-(1-piperidinyl)-1H-imidazo[4,5-c]pyridin-2-yl]-1,2,5-oxadiazol-3-amine;
1-[2-(4-Aminofurazan-3-yl)-1-ethyl-4-phenyl-1-H-imidazo[4,5-c]pyridin-7-yl]-1-(3-aminopyrrolidin-1-yl)methanone;
1-[2-(4-Aminofurazan-3-yl)-1-ethyl-4-thiophen-3-yl-1-H-imidazo[4,5-c]pyridin-7-yl]-1-(3-aminopyrrolidin-1-yl)methanone;
1-[2-(4-Aminofurazan-3-yl)-1-ethyl-4-pyridin-yl-1-H-imidazo[4,5-c]pyridin-7-yl]-1-(3-aminopyrrolidin-1-yl)methanone;
1-[2-(4-Aminofurazan-3-yl)-1-ethyl-4-pyridin-3-yl-1-H-imidazo[4,5-c]pyridin-7-yl]-1-(3-aminopyrrolidin-1-yl)methanone;
1-[2-(4-Aminofurazan-3-yl)-1-ethyl-4-furan-3-yl-1-H-imidazo[4,5-c]pyridin-7-yl]-1-(3-aminopyrrolidin-1-yl)methanone;
1-[2-(4-Amino-furazan-3-yl)-4-chloro-1-ethyl-1-H-imidazo[4,5-c]pyridin-7-yl]-1-(3-amino-pyrrolidin-1-yl)-methanone;
1-[2-(4-Amino-furazan-3-yl)-4-(1H-pyrrol-2-yl))-1-ethyl-1-H-imidazo[4,5-c]pyridin-7-yl]-1-(3-amino-pyrrolidin-1-yl)-methanone;
1-[2-(4-Amino-furazan-3-yl)-1-ethyl-4-(2-methoxyphenyl)-1H-imidazo[4,5-c]pyridin-7-yl]-1-(3-amino-pyrrolidin-1-yl)-methanone;
1-[2-(4-Amino-furazan-3-yl)-1-ethyl-4-(3-chloro-phenyl)-1H-imidazo[4,5-c]pyridin-7-yl]-1-(3-amino-pyrrolidin-1-yl)-methanone;
1-[2-(4-Amino-furazan-3-yl)-1-ethyl-4-furan-2-yl-1H-imidazo[4,5-c]pyridin-7-yl]-1-(3-amino-pyrrolidin-1-yl)-methanone;
2-(4-Amino-furazan-3-yl)-1-ethyl-4-phenyl-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(3-chloro-phenyl)-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(2,3-dichloro-phenyl)-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(2-chloro-phenyl)-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(2-hydroxy-phenyl)-1H-imidazo[4,5-c]pyridine-7-carboxylic acid [1-(4-chloro-benzyl)-2-hydroxy-ethyl]-amide;
2-(4-Amino-furazan-3-yl)-4-(3-chloro-phenyl)-1-ethyl-1H-imidazo[4,5-c]pyridine-7-carboxylic acid pyrrolidin-3-ylamide;
2-(4-Amino-furazan-3-yl)-4-phenyl-1-ethyl-1H-imidazo[4,5-c]pyridine-7-carboxylic acid pyrrolidin-3-ylamide;
2-(4-Amino-furazan-3-yl)-4-(5-chloro-thiophen-2-yl)-1-ethyl-1H-imidazo[4,5-c]pyridine-7-carboxylic acid pyrrolidin-3-ylamide;
2-(4-Amino-furazan-3-yl)-4-(2-amino-phenyl)-1-ethyl-1H-imidazo[4,5-c]pyridine-7-carboxylic acid pyrrolidin-3-ylamide;
2-(4-Amino-furazan-3-yl)-4-(3-amino-phenyl)-1-ethyl-1H-imidazo[4,5-c]pyridine-7-carboxylic acid pyrrolidin-3-ylamide;
2-(4-Amino-furazan-3-yl)-4-(3-bromo-phenyl)-1-ethyl-1H-imidazo[4,5-c]pyridine-7-carboxylic acid pyrrolidin-3-ylamide;
2-(4-Amino-furazan-3-yl)-4-(1-naphthalenyl)-1-ethyl-1H-imidazo[4,5-c]pyridine-7-carboxylic acid pyrrolidin-3-ylamide;
2-(4-Amino-furazan-3-yl)-4-(thiophen-2-yl)-1-ethyl-1H-imidazo[4,5-c]pyridine-7-carboxylic acid pyrrolidin-3-ylamide;
2-(4-Amino-furazan-3-yl)-4-(3,4-methylenedioxyphenyl)-1-ethyl-1H-imidazo[4,5-c]pyridine-7-carboxylic acid pyrrolidin-3-ylamide;
2-(4-Amino-furazan-3-yl)-4-(3,5-dichloro-phenyl)-1-ethyl-1H-imidazo[4,5-c]pyridine-7-carboxylic acid pyrrolidin-3-ylamide;
4-[7-[(3-amino-1-pyrrolidinyl)carbonyl]-4-(3-chlorophenyl)-1-(cyclopropylmethyl)-1H-imidazo[4,5-c]pyridin-2-yl]-1,2,5-oxadiazol-3-amine;
4-[7-[(3-amino-1-pyrrolidinyl)carbonyl]-4-(4-biphenylyl)-1-ethyl-1H-imidazo[4,5-c]pyridin-2-yl]-1,2,5-oxadiazol-3-amine;
4-[7-[(3-amino-1-pyrrolidinyl)carbonyl]-4-(2,4-dichlorophenyl)-1-ethyl-1H-imidazo[4,5-c]pyridin-2-yl]-1,2,5-oxadiazol-3-amine;
4-[7-[(3-amino-1-pyrrolidinyl)carbonyl]-1-ethyl-4-(phenylethynyl)-1H-imidazo[4,5-c]pyridin-2-yl]-1,2,5-oxadiazol-3-amine;
2-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol;
4-[7-[(3-amino-1-pyrrolidinyl)carbonyl]-4-(2-chlorophenyl)-1-ethyl-1H-imidazo[4,5-c]pyridin-2-yl]-1,2,5-oxadiazol-3-amine;
(2-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenyl)methanol;
2-{2-(4-amino-1,2,5-oxadiazol-3-yl)-7-[(3-amino-1-pyrrolidinyl)carbonyl]-1-ethyl-1H-imidazo[4,5-c]pyridin-4-yl}-4-chlorophenol;
4-(1-ethyl-7-{[3-(methylamino)-1-pyrrolidinyl]carbonyl}-4-phenyl-1H-imidazo[4,5-c]pyridin-2-yl)-1,2,5-oxadiazol-3-amine;
4-[7-[(3-amino-1-pyrrolidinyl)carbonyl]-1-ethyl-4-(4-methylphenyl)-1H-imidazo[4,5-c]pyridin-2-yl]-1,2,5-oxadiazol-3-amine;
4-[7-[(3-amino-1-pyrrolidinyl)carbonyl]-4-(2,5-dichlorophenyl)-1-ethyl-1H-imidazo[4,5-c]pyridin-2-yl]-1,2,5-oxadiazol-3-amine;
4-[7-[(3-amino-1-pyrrolidinyl)carbonyl]-4-(1-benzothien-2-yl)-1-ethyl-1H-imidazo[4,5-c]pyridin-2-yl]-1,2,5-oxadiazol-3-amine;
4-[1-ethyl-4-phenyl-7-(4-piperidinyloxy)-1H-imidazo[4,5-c]pyridin-2-yl]-1,2,5-oxadiazol-3-amine;
4-{7-[(3-amino-1-pyrrolidinyl)carbonyl]-1-ethyl-4-[4-(methyloxy)phenyl]-1H-imidazo[4,5-c]pyridin-2-yl}-1,2,5-oxadiazol-3-amine;
4-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol;
4-[7-[(3-amino-1-pyrrolidinyl)carbonyl]-4-(4-chlorophenyl)-1-ethyl-1H-imidazo[4,5-c]pyridin-2-yl]-1,2,5-oxadiazol-3-amine;
4-[4-(3-chlorophenyl)-1-ethyl-7-(4-piperidinyloxy)-1H-imidazo[4,5-c]pyridin-2-yl]-1,2,5-oxadiazol-3-amine;
2-(4-amino-1,2,5-oxadiazol-3-yl)-4-(3-chlorophenyl)-1-(cyclopropylmethyl)-N-{2-[(phenylmethyl)amino]ethyl}-1H-imidazo[4,5-c]pyridine-7-carboxamide;
3-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol;
4-{2-(4-amino-1,2,5-oxadiazol-3-yl)-7-[(3-amino-1-pyrrolidinyl)carbonyl]-1-ethyl-1H-imidazo[4,5-c]pyridin-4-yl}benzonitrile;
1-[2-(4-Amino-furazan-3-yl)-4-phenyl-1-piperidin-4yl-1-H-imidazo[4,5-c]pyridin-7-yl]-1-(3-amino-pyrrolidin-1-yl)-methanone;
4-(4-(3-chlorophenyl)-1-ethyl-7-{[3-(methylamino)-1-pyrrolidinyl]carbonyl}-1H-imidazo[4,5-c]pyridin-2-yl)-1,2,5-oxadiazol-3-amine;
4-(4-(2,5-dichlorophenyl)-1-ethyl-7-{[3-(methylamino)-1-pyrrolidinyl]carbonyl}-1H-imidazo[4,5-c]pyridin-2-yl)-1,2,5-oxadiazol-3-amine;
4-[4-(2,5-dichlorophenyl)-1-ethyl-7-(4-piperidinyloxy)-1H-imidazo[4,5-c]pyridin-2-yl]-1,2,5-oxadiazol-3-amine;
2-(4-amino-1,2,5-oxadiazol-3-yl)-4-(3-chlorophenyl)-1-(cyclopropylmethyl)-N-[3-(dimethylamino)propyl]-1H-imidazo[4,5-c]pyridine-7-carboxamide;
4-[7-[(3-amino-1-pyrrolidinyl)carbonyl]-1-ethyl-4-(1H-pyrrol-2-yl)-1H-imidazo[4,5-c]pyridin-2-yl]-1,2,5-oxadiazol-3-amine;
4-[7-[(3-amino-1-pyrrolidinyl)carbonyl]-4-(4-bromophenyl)-1-ethyl-1H-imidazo[4,5-c]pyridin-2-yl]-1,2,5-oxadiazol-3-amine;
4-[7-[(3-amino-1-pyrrolidinyl)carbonyl]-4-phenyl-1-(4-piperidinyl)-1H-imidazo[4,5-c]pyridin-2-yl]-1,2,5-oxadiazol-3-amine;
4-{7-[(4-aminobutyl)oxy]-1-ethyl-4-phenyl-1H-imidazo[4,5-c]pyridin-2-yl}-1,2,5-oxadiazol-3-amine;
4-{1-ethyl-4-phenyl-7-[(4-piperidinylmethyl)oxy]-1H-imidazo[4,5-c]pyridin-2-yl}-1,2,5-oxadiazol-3-amine;
4-{4-(3-chlorophenyl)-1-ethyl-7-[(4-piperidinylmethyl)oxy]-1H-imidazo[4,5-c]pyridin-2-yl}-1,2,5-oxadiazol-3-amine;
4-[7-[(4-aminobutyl)oxy]-4-(3-chlorophenyl)-1-ethyl-1H-imidazo[4,5-c]pyridin-2-yl]-1,2,5-oxadiazol-3-amine;
4-{7-[(2-aminoethyl)oxy]-1-ethyl-4-phenyl-1H-imidazo[4,5-c]pyridin-2-yl}-1,2,5-oxadiazol-3-amine;
4-{1-ethyl-4-phenyl-7-[(3-pyrrolidinylmethyl)oxy]-1H-imidazo[4,5-c]pyridin-2-yl}-1,2,5-oxadiazol-3-amine;
4-{7-[(3-aminopropyl)oxy]-1-ethyl-4-phenyl-1H-imidazo[4,5-c]pyridin-2-yl}-1,2,5-oxadiazol-3-amine;
4-(7-{[(2S)-2-amino-3-phenylpropyl]oxy}-1-ethyl-4-phenyl-1H-imidazo[4,5-c]pyridin-2-yl)-1,2,5-oxadiazol-3-amine;
4-[1-ethyl-4-phenyl-7-(3-piperidinyloxy)-1H-imidazo[4,5-c]pyridin-2-yl]-1,2,5-oxadiazol-3-amine;
2-(4-amino-1,2,5-oxadiazol-3-yl)-1-ethyl-N-methyl-N-(1-methyl-4-piperidinyl)-4-phenyl-1H-imidazo[4,5-c]pyridine-7-carboxamide;
N-{[2-(4-amino-1,2,5-oxadiazol-3-yl)-1-ethyl-4-phenyl-1H-imidazo[4,5-c]pyridin-7-yl]methyl}-N,1-dimethyl-4-piperidinamine;
4-(1-ethyl-4-phenyl-7-{[2-(4-piperidinyl)ethyl]oxy}-1H-imidazo[4,5-c]pyridin-2-yl)-1,2,5-oxadiazol-3-amine;
4-{1-(4-aminobutyl)-7-[(3-amino-1-pyrrolidinyl)carbonyl]-4-phenyl-1H-imidazo[4,5-c]pyridin-2-yl}-1,2,5-oxadiazol-3-amine;
4-(7-{[(2R)-2-amino-3-phenylpropyl]oxy}-1-ethyl-4-phenyl-1H-imidazo[4,5-c]pyridin-2-yl)-1,2,5-oxadiazol-3-amine;
4-{1-(4-aminobutyl)-7-[(3-amino-1-pyrrolidinyl)carbonyl]-4-phenyl-1H-imidazo[4,5-c]pyridin-2-yl}-1,2,5-oxadiazol-3-amine;
4-(1-(4-aminobutyl)-7-{[3-(methylamino)-1-pyrrolidinyl]carbonyl}-4-phenyl-1H-imidazo[4,5-c]pyridin-2-yl)-1,2,5-oxadiazol-3-amine;
4-{1-ethyl-7-[(4-methyl-1-piperazinyl)methyl]-4-phenyl-1H-imidazo[4,5-c]pyridin-2-yl}-1,2,5-oxadiazol-3-amine;
4-(1-ethyl-7-{[3-(methylamino)-1-pyrrolidinyl]methyl}-4-phenyl-1H-imidazo[4,5-c]pyridin-2-yl)-1,2,5-oxadiazol-3-amine;
(3-amino-2,2-dimethylpropyl){[2-(4-amino-1,2,5-oxadiazol-3-yl)-1-ethyl-4-phenyl-1H-imidazo[4,5-c]pyridin-7-yl]methyl}amine;
4-(7-{[3-(dimethylamino)-1-pyrrolidinyl]methyl}-1-ethyl-4-phenyl-1H-imidazo[4,5-c]pyridin-2-yl)-1,2,5-oxadiazol-3-amine;
4-(1-ethyl-7-{[2-(methylamino)ethyl]oxy}-4-phenyl-1H-imidazo[4,5-c]pyridin-2-yl)-1,2,5-oxadiazol-3-amine;
4-[1-ethyl-4-phenyl-7-({2-[(phenylmethyl)amino]ethyl}oxy)-1H-imidazo[4,5-c]pyridin-2-yl]-1,2,5-oxadiazol-3-amine;
4-{1-ethyl-4-phenyl-7-[(3-piperidinylmethyl)oxy]-1H-imidazo[4,5-c]pyridin-2-yl}-1,2,5-oxadiazol-3-amine;
4-{7-[(5-aminopentyl)oxy]-1-ethyl-4-phenyl-1H-imidazo[4,5-c]pyridin-2-yl}-1,2,5-oxadiazol-3-amine;
4-(7-{[3-(dimethylamino)-2,2-dimethylpropyl]oxy}-1-ethyl-4-phenyl-1H-imidazo[4,5-c]pyridin-2-yl)-1,2,5-oxadiazol-3-amine;
1-(4-aminobutyl)-2-(4-amino-1,2,5-oxadiazol-3-yl)-4-phenyl-N-{2-[(phenylmethyl)amino]ethyl}-1H-imidazo[4,5-c]pyridine-7-carboxamide;
2-(4-amino-1,2,5-oxadiazol-3-yl)-1-(1-methylethyl)-4-phenyl-N-3-pyrrolidinyl-1H-imidazo[4,5-c]pyridine-7-carboxamide;
4-[7-{[3-(methylamino)-1-pyrrolidinyl]carbonyl}-1-(1-methylethyl)-4-phenyl-1H-imidazo[4,5-c]pyridin-2-yl]-1,2,5-oxadiazol-3-amine;
4-(7-{[(3S)-3-amino-1-pyrrolidinyl]methyl}-1-ethyl-4-phenyl-1H-imidazo[4,5-c]pyridin-2-yl)-1,2,5-oxadiazol-3-amine;
4-[1-ethyl-7-(hexahydro-1H-1,4-diazepin-1-ylmethyl)-4-phenyl-1H-imidazo[4,5-c]pyridin-2-yl]-1,2,5-oxadiazol-3-amine;
4-[1-ethyl-4-phenyl-7-(1-piperazinylmethyl)-1H-imidazo[4,5-c]pyridin-2-yl]-1,2,5-oxadiazol-3-amine;
4-(7-{[2-(dimethylamino)ethyl]oxy}-1-ethyl-4-phenyl-1H-imidazo[4,5-c]pyridin-2-yl)-1,2,5-oxadiazol-3-amine;
4-(1-ethyl-4-phenyl-7-{[(2S)-2-pyrrolidinylmethyl]oxy}-1H-imidazo[4,5-c]pyridin-2-yl)-1,2,5-oxadiazol-3-amine;
4-(1-ethyl-4-phenyl-7-{[(2R)-2-pyrrolidinylmethyl]oxy}-1H-imidazo[4,5-c]pyridin-2-yl)-1,2,5-oxadiazol-3-amine;
2-(4-amino-1,2,5-oxadiazol-3-yl)-N-(3-aminopropyl)-1-(1-methylethyl)-4-phenyl-1H-imidazo[4,5-c]pyridine-7-carboxamide;
2-(4-amino-1,2,5-oxadiazol-3-yl)-1-(1-methylethyl)-4-phenyl-N-2-propen-1-yl-1H-imidazo[4,5-c]pyridine-7-carboxamide;
2-(4-amino-1,2,5-oxadiazol-3-yl)-1-ethyl-N-[3-(4-morpholinyl)propyl]-4-phenyl-1H-imidazo[4,5-c]pyridine-7-carboxamide;
2-(4-amino-1,2,5-oxadiazol-3-yl)-1-ethyl-N-[2-(1H-imidazol-4-yl)ethyl]-4-phenyl-1H-imidazo[4,5-c]pyridine-7-carboxamide,
2-(4-amino-1,2,5-oxadiazol-3-yl)-1-ethyl-N-[3-(4-methyl-1-piperazinyl)propyl]-4-phenyl-1H-imidazo[4,5-c]pyridine-7-carboxamide;
4-[7-[(3-aminopropyl)oxy]-4-(2-chlorophenyl)-1-ethyl-1H-imidazo[4,5-c]pyridin-2-yl]-1,2,5-oxadiazol-3-amine;
4-[7-[(3-aminopropyl)oxy]-4-(3-chlorophenyl)-1-ethyl-1H-imidazo[4,5-c]pyridin-2-yl]-1,2,5-oxadiazol-3-amine;
4-[7-[(3-aminopropyl)oxy]-4-(4-chlorophenyl)-1-ethyl-1H-imidazo[4,5-c]pyridin-2-yl]-1,2,5-oxadiazol-3-amine;
4-{7-[(3-aminopropyl)oxy]-4-[5-chloro-2-(methyloxy)phenyl]-1-ethyl-1H-imidazo[4,5-c]pyridin-2-yl}-1,2,5-oxadiazol-3-amine;
N-(1-{[2-(4-amino-1,2,5-oxadiazol-3-yl)-1-ethyl-4-phenyl-1H-imidazo[4,5-c]pyridin-7-yl]carbonyl}-3-pyrrolidinyl)-N-methylacetamide;
2-(4-amino-1,2,5-oxadiazol-3-yl)-N-[3-(dimethylamino)propyl]-1-ethyl-4-phenyl-1H-imidazo[4,5-c]pyridine-7-carboxamide;
2-{2-(4-amino-1,2,5-oxadiazol-3-yl)-7-[(3-aminopropyl)oxy]-1-ethyl-1H-imidazo[4,5-c]pyridin-4-yl}-4-chlorophenol;
4-[7-[(3-aminopropyl)oxy]-1-ethyl-4-(2-pyridinyl)-1H-imidazo[4,5-c]pyridin-2-yl]-1,2,5-oxadiazol-3-amine;
4-(7-{[3-(dimethylamino)propyl]oxy}-1-ethyl-4-phenyl-1H-imidazo[4,5-c]pyridin-2-yl)-1,2,5-oxadiazol-3-amine;
4-(1-ethyl-7-{[3-(4-morpholinyl)propyl]oxy}-4-phenyl-1H-imidazo[4,5-c]pyridin-2-yl)-1,2,5-oxadiazol-3-amine;
, 2-(4-amino-1,2,5-oxadiazol-3-yl)-1-cyclopentyl-4-phenyl-N-3-pyrrolidinyl-1H-imidazo[4,5-c]pyridine-7-carboxamide;
4-{7-[(3-amino-1-pyrrolidinyl)carbonyl]-1-cyclopentyl-4-phenyl-1H-imidazo[4,5-c]pyridin-2-yl}-1,2,5-oxadiazol-3-amine;
4-(1-cyclopentyl-7-{[3-(methylamino)-1-pyrrolidinyl]carbonyl}-4-phenyl-1H-imidazo[4,5-c]pyridin-2-yl)-1,2,5-oxadiazol-3-amine;
4-(1-ethyl-7-{[3-(methylamino)propyl]oxy}-4-phenyl-1H-imidazo[4,5-c]pyridin-2-yl)-1,2,5-oxadiazol-3-amine;
4-{1-ethyl-7-[(3-hydrazinopropyl)oxy]-4-phenyl-1H-imidazo[4,5-c]pyridin-2-yl}-1,2,5-oxadiazol-3-amine;
2-[(3-{[2-(4-amino-1,2,5-oxadiazol-3-yl)-1-ethyl-4-phenyl-1H-imidazo[4,5-c]pyridin-7-yl]oxy}propyl)amino]ethanol;
4-(1-ethyl-7-{[3-(hydroxyamino)propyl]oxy}-4-phenyl-1H-imidazo[4,5-c]pyridin-2-yl)-1,2,5-oxadiazol-3-amine;
(3R)-1-{[2-(4-amino-1,2,5-oxadiazol-3-yl)-1-ethyl-4-phenyl-1H-imidazo[4,5-c]pyridin-7-yl]carbonyl}-3-pyrrolidinol;
2-(4-amino-1,2,5-oxadiazol-3-yl)-N-[3-(diethylamino)propyl]-1-ethyl-4-phenyl-1H-imidazo[4,5-c]pyridine-7-carboxamide;
2-(4-amino-1,2,5-oxadiazol-3-yl)-1-ethyl-N-[3-(2-methyl-1-piperidinyl)propyl]-4-phenyl-1H-imidazo[4,5-c]pyridine-7-carboxamide;
4-(1-methyl-7-{[3-(methylamino)-1-pyrrolidinyl]carbonyl}-4-phenyl-1H-imidazo[4,5-c]pyridin-2-yl)-1,2,5-oxadiazol-3-amine;
4-{7-[(3-amino-1-pyrrolidinyl)carbonyl]-1-methyl-4-phenyl-1H-imidazo[4,5-c]pyridin-2-yl}-1,2,5-oxadiazol-3-amine;
4-(1-butyl-7-{[3-(methylamino)-1-pyrrolidinyl]carbonyl}-4-phenyl-1H-imidazo[4,5-c]pyridin-2-yl)-1,2,5-oxadiazol-3-amine;
4-{7-[(3-amino-1-pyrrolidinyl)carbonyl]-1-butyl-4-phenyl-1H-imidazo[4,5-c]pyridin-2-yl}-1,2,5-oxadiazol-3-amine;
4-[7-[(3-amino-1-pyrrolidinyl)carbonyl]-1-(4-fluorophenyl)-4-phenyl-1H-imidazo[4,5-c]pyridin-2-yl]-1,2,5-oxadiazol-3-amine;
N-(2-aminoethyl)-2-(4-amino-1,2,5-oxadiazol-3-yl)-1-(4-fluorophenyl)-4-phenyl-1H-imidazo[4,5-c]pyridine-7-carboxamide;
4-{1-(4-aminophenyl)-7-[(3-amino-1-pyrrolidinyl)carbonyl]-4-phenyl-1H-imidazo[4,5-c]pyridin-2-yl}-1,2,5-oxadiazol-3-amine;
1-{[2-(4-amino-1,2,5-oxadiazol-3-yl)-1-ethyl-4-phenyl-1H-imidazo[4,5-c]pyridin-7-yl]oxy}-3-(4-morpholinyl)-2-propanol;
N-[2-(4-amino-1,2,5-oxadiazol-3-yl)-1-ethyl-4-phenyl-1H-imidazo[4,5-c]pyridin-7-yl]-4-piperidinecarboxamide;
4-[7-{[3-(dimethylamino)-1-pyrrolidinyl]carbonyl}-4-phenyl-1-(2,2,2-trifluoroethyl)-1H-imidazo[4,5-c]pyridin-2-yl]-1,2,5-oxadiazol-3-amine;
4-(1-ethyl-7-{[2-(4-morpholinyl)ethyl]oxy}-4-phenyl-1H-imidazo[4,5-c]pyridin-2-yl)-1,2,5-oxadiazol-3-amine;
4-(1-ethyl-4-phenyl-7-{[3-(1-piperidinyl)propyl]oxy}-1H-imidazo[4,5-c]pyridin-2-yl)-1,2,5-oxadiazol-3-amine trifluoroacetate;
2-(4-amino-1,2,5-oxadiazol-3-yl)-1-ethyl-N-[2-(1-methyl-2-pyrrolidinyl)ethyl]-4-phenyl-1H-imidazo[4,5-c]pyridine-7-carboxamide;
1-(1-{[2-(4-amino-1,2,5-oxadiazol-3-yl)-1-ethyl-4-phenyl-1H-imidazo[4,5-c]pyridin-7-yl]carbonyl}-4-piperidinyl)-1,3-dihydro-2H-benzimidazol-2-one;
1-{[2-(4-amino-1,2,5-oxadiazol-3-yl)-1-ethyl-4-phenyl-1H-imidazo[4,5-c]pyridin-7-yl]carbonyl}-3-piperidinecarboxamide;
(2-aminoethyl)(2-{[2-(4-amino-1,2,5-oxadiazol-3-yl)-1-ethyl-4-phenyl-1H-imidazo[4,5-c]pyridin-7-yl]oxy}ethyl)amine;
4-(1-ethyl-4-phenyl-7-{[2-(1-piperazinyl)ethyl]oxy}-1H-imidazo[4,5-c]pyridin-2-yl)-1,2,5-oxadiazol-3-amine;
4-(7-{[2-(4-acetyl-1-piperazinyl)ethyl]oxy}-1-ethyl-4-phenyl-1H-imidazo[4,5-c]pyridin-2-yl)-1,2,5-oxadiazol-3-amine trifluoroacetate;
4-(1-ethyl-7-{[3-(4-methyl-1-piperazinyl)propyl]oxy}-4-phenyl-1H-imidazo[4,5-c]pyridin-2-yl)-1,2,5-oxadiazol-3-amine;
4-(1-ethyl-4-phenyl-7-{[3-(1-piperazinyl)propyl]oxy}-1H-imidazo[4,5-c]pyridin-2-yl)-1,2,5-oxadiazol-3-amine;
4-(1-ethyl-4-phenyl-7-{[2-(1-piperidinyl)ethyl]oxy}-1H-imidazo[4,5-c]pyridin-2-yl)-1,2,5-oxadiazol-3-amine trifluoroacetate;
(3-{[2-(4-amino-1,2,5-oxadiazol-3-yl)-1-ethyl-4-phenyl-1H-imidazo[4,5-c]pyridin-7-yl]oxy}propyl)[2-(dimethylamino)ethyl]methylamine;
3-[(3-{[2-(4-amino-1,2,5-oxadiazol-3-yl)-1-ethyl-4-phenyl-1H-imidazo[4,5-c]pyridin-7-yl]oxy}propyl)amino]-1,2-propanediol;
N-(3-amino-2-hydroxypropyl)-2-(4-amino-1,2,5-oxadiazol-3-yl)-1-ethyl-4-phenyl-1H-imidazo[4,5-c]pyridine-7-carboxamide;
N-(2-amino-3-hydroxypropyl)-2-(4-amino-1,2,5-oxadiazol-3-yl)-1-ethyl-4-phenyl-1H-imidazo[4,5-c]pyridine-7-carboxamide;
N-(3-{2-(4-amino-1,2,5-oxadiazol-3-yl)-7-[(3-aminopropyl)oxy]-1-ethyl-1H-imidazo[4,5-c]pyridin-4-yl}phenyl)-N′-phenylurea;
3-{[2-(4-amino-1,2,5-oxadiazol-3-yl)-1-ethyl-4-phenyl-1H-imidazo[4,5-c]pyridin-7-yl]oxy}-1-propanol;
(4-{[2-(4-amino-1,2,5-oxadiazol-3-yl)-1-ethyl-4-phenyl-1H-imidazo[4,5-c]pyridin-7-yl]carbonyl}-2-piperazinyl)methanol;
4-[1-ethyl-7-({3-[(methyloxy)methyl]-1-piperazinyl}carbonyl)-4-phenyl-1H-imidazo[4,5-c]pyridin-2-yl]-1,2,5-oxadiazol-3-amine;
4-(7-{[3-({[2,4-bis(methyloxy)phenyl]methyl}amino)propyl]oxy}-1-ethyl-4-phenyl-1H-imidazo[4,5-c]pyridin-2-yl)-1,2,5-oxadiazol-3-amine;
(2S)-2-[(3-{[2-(4-amino-1,2,5-oxadiazol-3-yl)-1-ethyl-4-phenyl-1H-imidazo[4,5-c]pyridin-7-yl]oxy}propyl)amino]-4-methyl-1-pentanol;
diethyl 1-[2-(4-amino-1,2,5-oxadiazol-3-yl)-1-ethyl-7-hydroxy-4-phenyl-1H-imidazo[4,5-c]pyridin-6-yl]-1,2-hydrazinedicarboxylate; and
4-(2-(4-amino-1,2,5-oxadiazol-3-yl)-1-ethyl-7-{[3-({2-[4-(methyloxy)phenyl]ethyl}amino)propyl]oxy}-1H-imidazo[4,5-c]pyridin-4-yl)-2-methyl-3-butyn-2-ol;
   and/or pharmaceutically acceptable salts, hydrates, solvates and pro-drugs thereof.

Compounds of Formula (I) are included in the pharmaceutical compositions of the invention and used in the methods of the invention. The compound 4-[1-Ethyl-7-(piperidin-4-yloxy)-1H-imidazo[4,5-c]pyridin-2-yl]-furazan-3-ylamine is also included in the methods of the invention.

By the term “protected hydroxy” or “protected —OH” as used herein, is meant the alcoholic or carboxylic-OH groups which can be protected by conventional blocking groups in the art such as described in “Protective Groups In Organic Synthesis” by Theodora W. Greene, Wiley-Interscience, 1981, New York. Compounds containing protected hydroxy groups may also be useful as intermediates in the preparation of the pharmaceutically active compounds of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, furan, pyrrole, pyrazole, imidazole, indole, indene, pyrazine, 1,3-dihydro-2H-benzimidazol, benzothiohpene and tetrazole.

The term “substituted” as used herein, unless otherwise defined, is meant that the subject chemical moiety has one or more substituents selected from the group consisting of: —CO₂R²⁰, aryl, —C(O)NHS(O)₂R²⁰, —NHS(O)₂R²⁰, hydroxyalkyl, alkoxy, —C(O)NR²¹R²², acyloxy, alkyl, amino, methylamino, nitrile, acetamide, urea, alkylurea, benzoate, sulfonamide, benzoateurea, alkoxyalkylamide, alkoxyC₁-C₁₂aryl, triphenylalkyl, cyclohexyl, C₁-C₁₂arylalkylurea, C₁-C₁₂aryl, haloC₁-C₁₂aryl, dimethylamino, N-acylamino, hydroxy, —(CH₂)_{g}C(O)OR²³, —S(O)ₙR²³, nitro, tetrazole, cyano, oxo, halogen and trifluoromethyl, where g is 0-6, R²³ is hydrogen or alkyl, R²⁰ is selected form hydrogen, C₁-C₄alkyl, aryl and trifluoromethyl, and R²¹ and R²² are independently selected form hydrogen, C₁-C₄alkyl, aryl and trifluoromethyl, and n is 0-2.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, 4-hydroxy-cyclohexyl, 2-ethylcyclohexyl, propyl 4-methoxycyclohexyl, 4-methoxycyclohexyl, 4-carboxycyclohexyl, cyclopropyl and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidyl, piperidine, pyrrolidine, 3-methylaminopyrrolidine, piperazinly, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms. Examples of alkyl and substituted alkyl substituents as used herein include: —CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C═C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃₁—CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

All publications, including but not limited to patents and patent applications, cited in this specification are herein incorporated by reference as though fully set forth.

As used herein, the term “effective amount” and derivatives thereof means that amount of a drug or pharmaceutical agent that will elicit the biological or medical response of a tissue, system, animal or human that is being sought, for instance, by a researcher or clinician. Furthermore, the term “therapeutically effective amount” and derivatives thereof means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I, II, III and IV are prepared as shown in Schemes 1 to 13 below, or by analogous methods, wherein the ‘Het’ and ‘R’ substituents are as defined in Formulas I, II, III and IV respectively and provided that the ‘Het’ and ‘R’ substituents do not include any such substituents that render inoperative the processes of Schemes 1 to 13. All of the starting materials are commercially available or are readily made from commercially available starting materials by those of skill in the art.

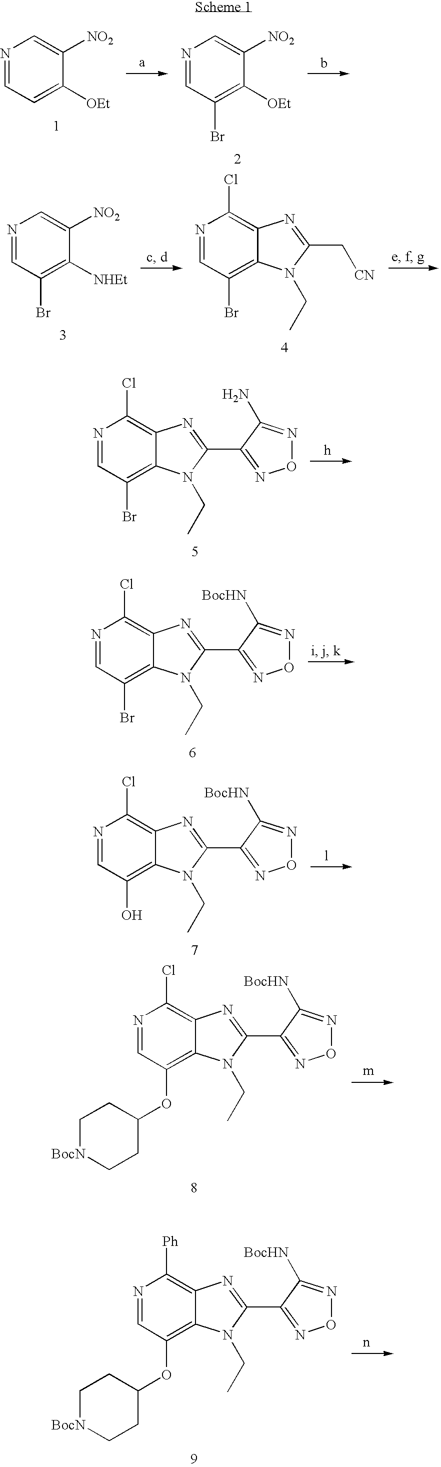

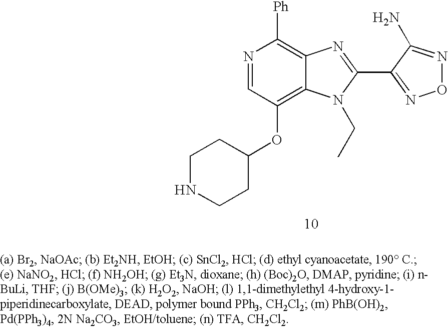

Compounds of Formula (I) can be prepared in a manner analogous to those shown in Scheme 1. Bromination of 3-nitro-4-ethoxy pyridine (1-Scheme 1) using bromine buffered in sodium acetate gives 3-bromo-4-(ethyloxy)-5-nitropyridine (2-Scheme 1). Other alternative methods exist and are known to those skilled in the art for carrying out this transformation. A compilation of these methods can be found in standard organic synthesis texts such as Larock, “Comprehensive Organic Transformations,” VCH, N.Y. (1989). The ethoxy group is then displaced by a primary amine such as ethyl amine in a polar solvent such as ethanol to give compounds such as 3-Scheme 1. In the case liquid amines, the reaction can be carried out in the absence of solvent. The reduction of the nitro group with concomitant introduction of the chloro group is achieved using tin (II) chloride according to the method described by Kelley et al. J. Med. Chem. 1995, 38(20), 4131-34. The corresponding 5-bromo-2-chloro diaminopyridine is condensed with an appropriate acid or ester such as ethyl cyanoacetate. Continued heating affects a cyclodehydration reaction to give imidazopyridines such as 4-Scheme 1. Reaction with NaNO₂ in concentrated HCl following by reaction with hydroxylamine gives a bis-oxime that cyclodehydrates in the presence of an appropriate base such as triethylamine to give an aminofurazan such as 5-Scheme 1. The amino group is protected by reacting with di-t-butyldicarbonate to give the corresponding t-butyl carbamate, 6-Scheme 1. Many different protecting groups are available to one skilled in the art and can be used here as long as they do not interfere with the processes listed herein. The hydroxyl group is introduced by generating an aryl anion by halogen-metal exchange using a suitable base such as n-butyl lithium, reacting the anion with an appropriate boron electrophile such as trimethyl borate and oxidizing the resulting aryl boronate with an appropriate oxidizing agent such as hydrogen peroxide in aqueous base to give imidazopyridinols such as 7-Scheme 1. Etherification of the imidazopyridinol is carried out with an appropriate alcohol such as 1,1-dimethylethyl 4-hydroxy-1-piperidinecarboxylate using the methods described by Mitsunobu, Synthesis 1981, 1 to give ethers such as 8-Scheme 1. Subsequent reaction with an aryl boronic acid such as phenyl boronic acid in the presence of a catalyst, preferably tetrakistriphenylphosphino palladium and a base such as sodium carbonate or triethylamine in a suitable solvent mixture such as toluene and ethanol gives the corresponding aryl compound such as 9-Scheme 1. Removal of the protecting groups is achieved using a protic or Lewis acid such as trifluoroacetic acid in a polar solvent such as methylene chloride giving compounds of Formula (I) such as 10-Scheme 1.

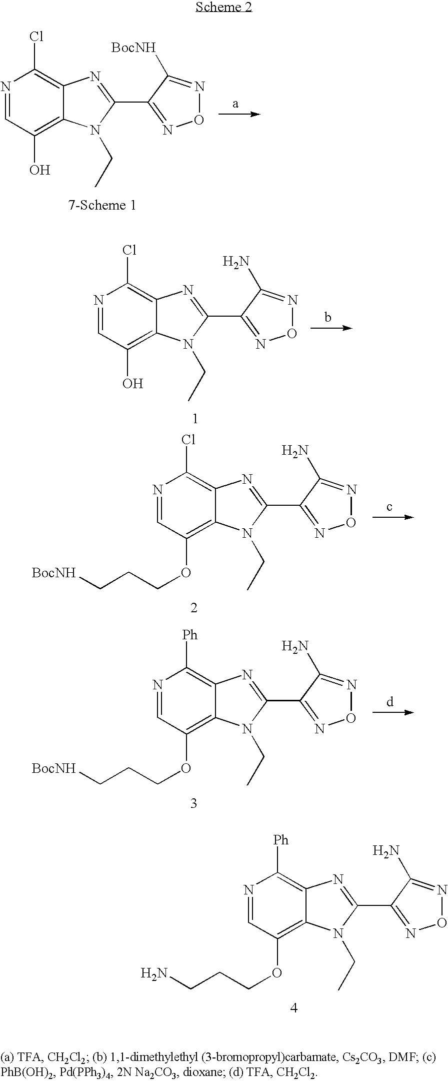

Alternatively, compounds of Formula (I) can be prepared starting with an intermediate such as 7-Scheme 1. Removal of the protecting groups using a protic or Lewis acid such as trifluoroacetic acid in a polar solvent such as methylene chloride gives an imidazopyridinol such as 1-Scheme 2. The phenolic —OH is deprotonated using a mild base such as Cs₂CO₃ and then alkylated with an appropriate electrophile such as 1,1-dimethylethyl (3-bromopropyl)carbamate in a polar solvent such as DMF to give the corresponding ether such as 2-Scheme 2. Subsequent reaction with an aryl boronic acid such as phenyl boronic acid in the presence of a catalyst, preferably tetrakistriphenylphosphino palladium and a base such as sodium carbonate or triethylamine in a suitable solvent such as dioxane gives the corresponding aryl compound such as 3-Scheme 2. Removal of the protecting groups is achieved using a protic or Lewis acid such as trifluoroacetic acid in a polar solvent such as methylene chloride giving compounds of Formula (I) such as 4-Scheme 2.

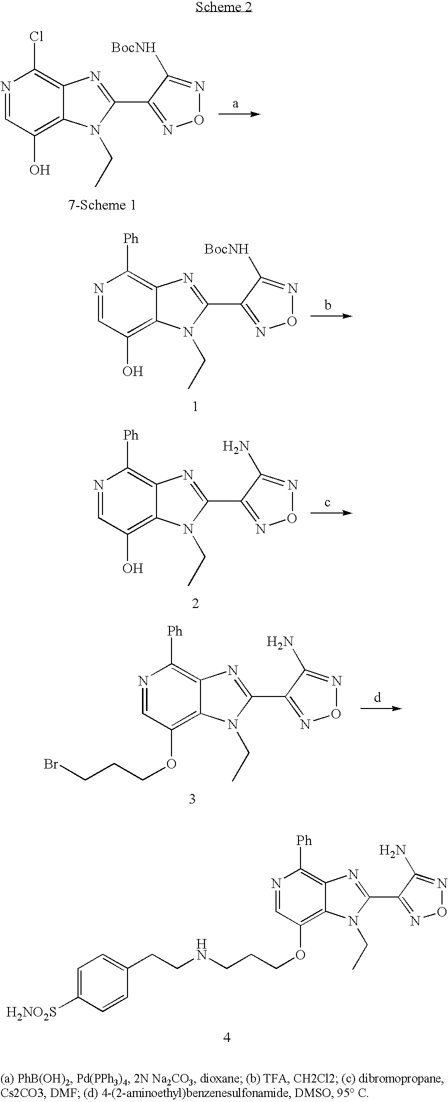

Alternatively, compounds of Formula (I) can be prepared starting from an intermediate such as 7-Scheme 1. Reaction with an aryl boronic acid such as phenyl boronic acid in the presence of a catalyst, preferably tetrakistriphenylphosphino palladium and a base such as sodium carbonate or triethylamine in a suitable solvent such as dioxane gives the corresponding aryl compound such as 1-Scheme 3. Removal of the protecting groups using a protic or Lewis acid such as trifluoroacetic acid in a polar solvent such as methylene chloride gives an imidazopyridinol such as 2-Scheme 3. The phenolic —OH is deprotonated using a mild base such as Cs₂CO₃ and then alkylated with an appropriate electrophile such as dibromopropane in a polar solvent such as DMF to give the corresponding ether such as 3-Scheme 3. Heating with an appropriate nucleophile such as 4-(2-aminoethyl)benzenesulfonamide in polar solvent such as dimethyl sulfoxide gives compounds of Formula (I) such as 4-Scheme 3.

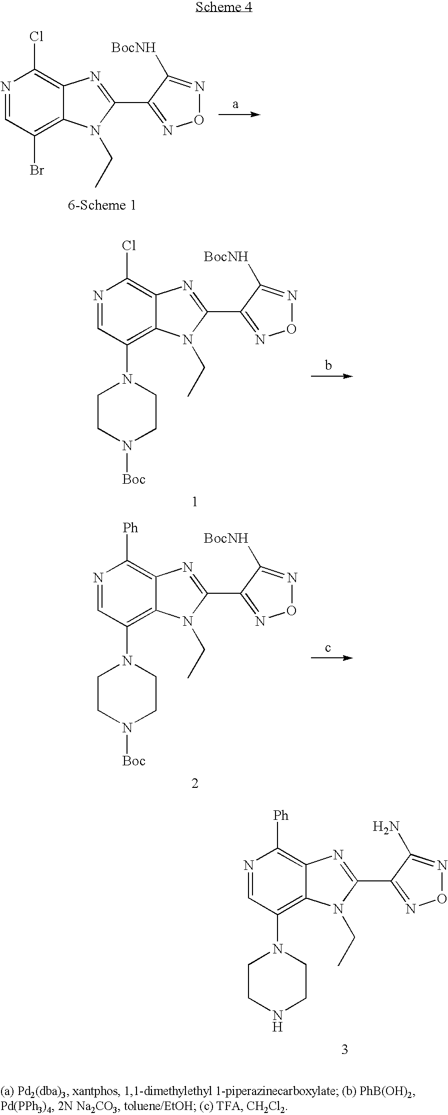

Alternatively, compounds of Formula (I) can be prepared starting from intermediate 6-Scheme 1. Reaction with an amine such as 1,1-dimethylethyl 1-piperazinecarboxylate in the presence of a catalyst, preferably Pd₂(dba)₃ following the method of Buchwald et al. J. Org. Chem. 2003, 68(25), 9563-73 gives the corresponding compound such as 1-Scheme 4. Reaction with an aryl boronic acid such as phenyl boronic acid in the presence of a catalyst, preferably tetrakistriphenylphosphino palladium and a base such as sodium carbonate or triethylamine in a suitable solvent mixture such as toluene and EtOH gives the corresponding aryl compound such as 2-Scheme 4. Removal of the protecting groups is achieved using a protic or Lewis acid such as trifluoroacetic acid in a polar solvent such as methylene chloride giving compounds of Formula (I) such as 3-Scheme 4.

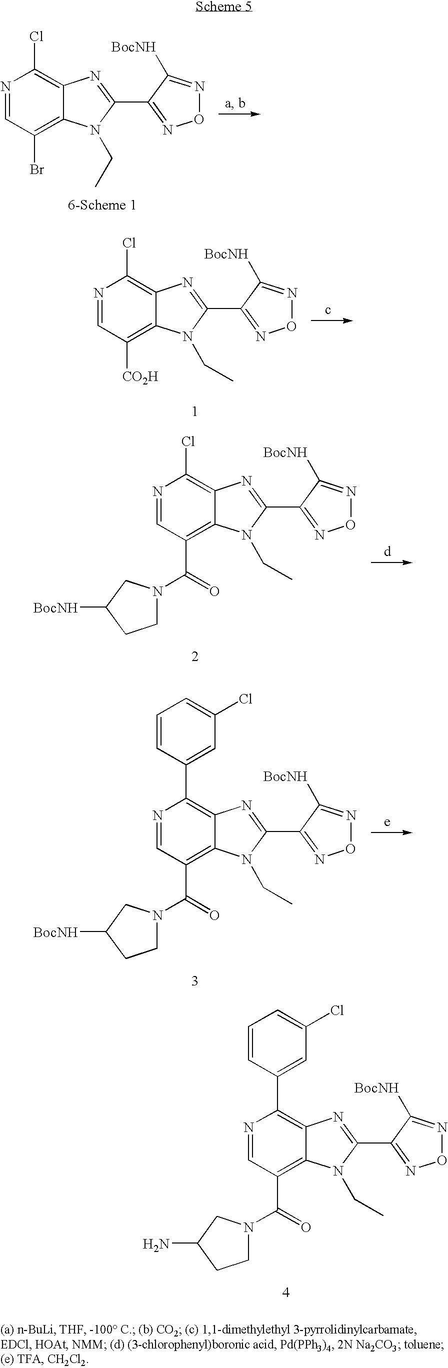

Alternatively, compounds of Formula (I) can prepared starting from intermediate 6-Scheme 1. Selective halogen metal exchange of the bromine using a suitable base such as n-BuLi in a suitable solvent such as THF at low temperatures generates the aryl anion which is quenched with CO₂ to give the corresponding carboxylic acid such as 1-Scheme 5. The acid is activated with a suitable reagent such as EDCI in the presence of a base such as N-methyl morpholine and is condensed with a suitable amine such as 1,1-dimethylethyl 3-pyrrolidinylcarbamate to give the corresponding amide such as 3-Scheme 5. Other alternative methods exist and are known to those skilled in the art for carrying out this transformation. A compilation of these methods can be found in standard organic synthesis texts such as Larock, “Comprehensive Organic Transformations,” VCH, N.Y. (1989). Reaction with an aryl boronic acid such as (3-chlorophenyl)boronic acid in the presence of a catalyst, preferably tetrakistriphenylphosphino palladium and a base such as sodium carbonate or triethylamine in a suitable solvent such as toluene gives compounds such as 3-Scheme 5. Removal of the protecting groups is achieved using a protic or Lewis acid such as trifluoroacetic acid in a polar solvent such as methylene chloride giving compounds of Formula (I) such as 4-Scheme 5.

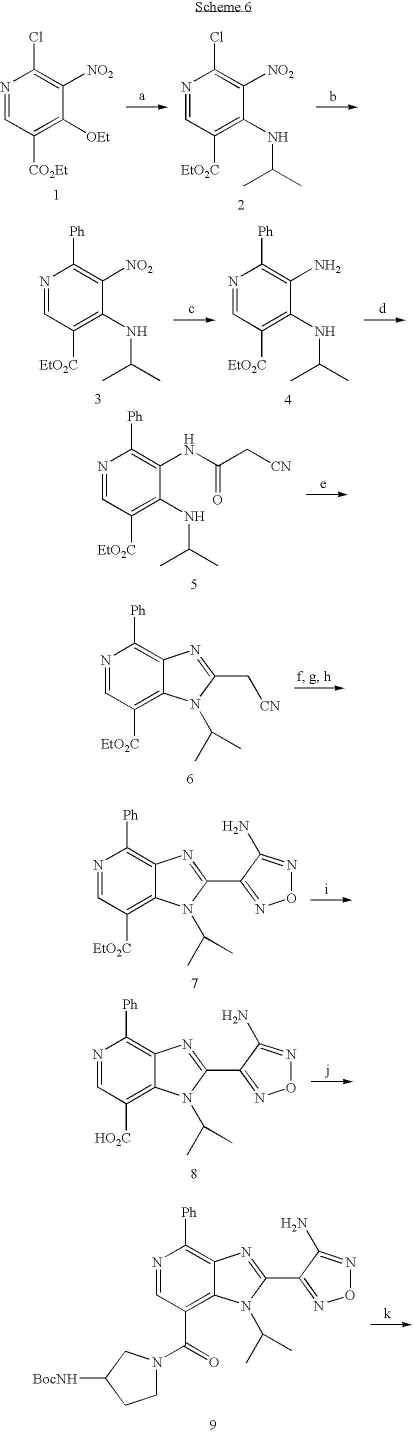

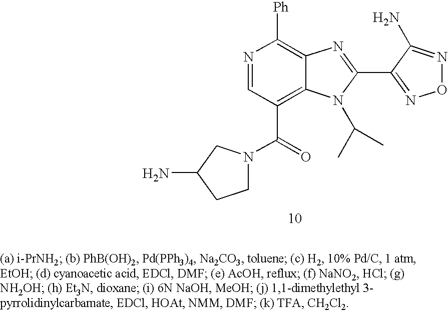

Alternatively, compounds of Formula (I) can be prepared in a manner analogous to that shown in Scheme 6. Ethyl 4,6-dichloro-5-nitro-3-pyridinecarboxylate, prepared according to Sanchez et al. J. Heterocycl. Chem. 1993, 30(4), 855-860, is reacted with an appropriate primary amine such as isopropyl amine to give a secondary amine such as 2-Scheme 6. Reaction with an aryl boronic acid such as phenylboronic acid in the presence of a catalyst, preferably tetrakistriphenylphosphino palladium and a base such as sodium carbonate or triethylamine in a suitable solvent such as toluene gives the corresponding aryl compound such as 3-Scheme 6. The nitro group is reduced using hydrogen gas at a suitable pressure such as 1 atmosphere in the presence of a suitable catalyst such as 10% Pd on carbon in a suitable solvent such as EtOH to give the corresponding diaminopyridine such as 4-Scheme 6. Other alternative methods exist and are known to those skilled in the art for carrying out this transformation. A compilation of these methods can be found in standard organic synthesis texts such as Larock, “Comprehensive Organic Transformations,” VCH, N.Y. (1989). The pyridyldiamine is condensed with cyanoacetic acid that has been activated by a suitable reagent such as EDCI in a polar solvent such as DMF. Heating the resulting amide such as 5-Scheme 6 in an acidic solvent such as acetic acid affects a cyclodehydration reaction to give the corresponding imidazopyridine such as 6-Scheme 6. Reaction with NaNO₂ in concentrated HCl following by reaction with hydroxylamine gives a bis-oxime that cyclodehydrates in the presence of an appropriate base such as triethylamine to give an aminofurazan such as 7-Scheme 6. Saponification of the ester using a base such as 6N NaOH in a suitable polar solvent such as MeOH gives the corresponding acid such as 8-Scheme 6. The acid is activated by suitable reagents such as EDCI and HOAT in the presence of a suitable base such as N-methyl morpholine in a polar solvent such as DMF and condensed with an appropriate amine such as 1,1-dimethylethyl 3-pyrrolidinylcarbamate to give the corresponding amide such as 9-Scheme 6. The protecting groups are removed using a protic or Lewis acid such as trifluoroacetic acid in a polar solvent such as methylene chloride to give compounds of Formula (I) such as 10-Scheme 6.

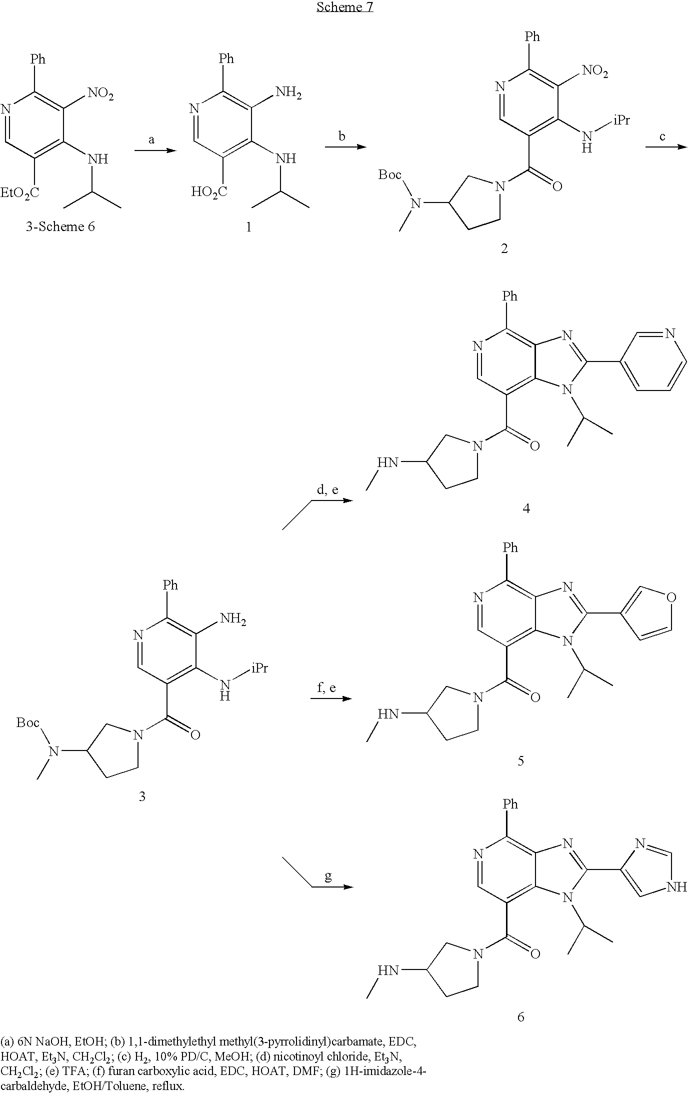

Alternatively, compounds of Formula (I) can be prepared starting with intermediate 3-Scheme 6. Saponification of the ester using a base such as 6N NaOH in a suitable polar solvent such as EtOH gives the corresponding acid such as 1-Scheme 7. The acid is activated by suitable reagents such as EDC and HOAT in the presence of a suitable base such as Et₃N in a polar solvent such as CH₂Cl₂ and condensed with an appropriate amine such as 1,1-dimethylethyl methyl(3-pyrrolidinyl)carbamate to give the corresponding amide such as 2-Scheme 7. The nitro group is reduced using hydrogen gas at a suitable pressure such as 1 atmosphere in the presence of a suitable catalyst such as 10% Pd on carbon in a suitable solvent such as MeOH to give the corresponding diaminopyridine such as 3-Scheme 7. The pyridyldiamine is condensed with a suitable acid chloride such as nicotinoyl chloride in the presence of a suitable base such as Et₃N in a suitable solvent such as CH₂Cl₂. The resulting amide is heated in the presence of a Lewis or protic acid such as TFA to affect a cyclodehydration with concomitant removal of the protecting groups to give compounds of Formula (I) such as 4-Scheme 7. Alternatively, a suitable diaminopyridine such as 3-Scheme 7 is condensed with a suitable acid such as furan carboxylic acid that has been activated by a suitable reagents such as EDC and HOAT in a polar solvent such as DMF. The resulting amide is heated in the presence of a Lewis or protic acid such as TFA to affect a cyclodehydration to give compounds of Formula (I) such as 5-Scheme 7. Alternatively, the pyridyldiamine is heated with a suitable aldehyde such as 1H-imidazole-4-carbaldehyde in an suitable solvent system such as EtOH/toluene to give compounds of Formula (I) such as 6-Scheme 7.

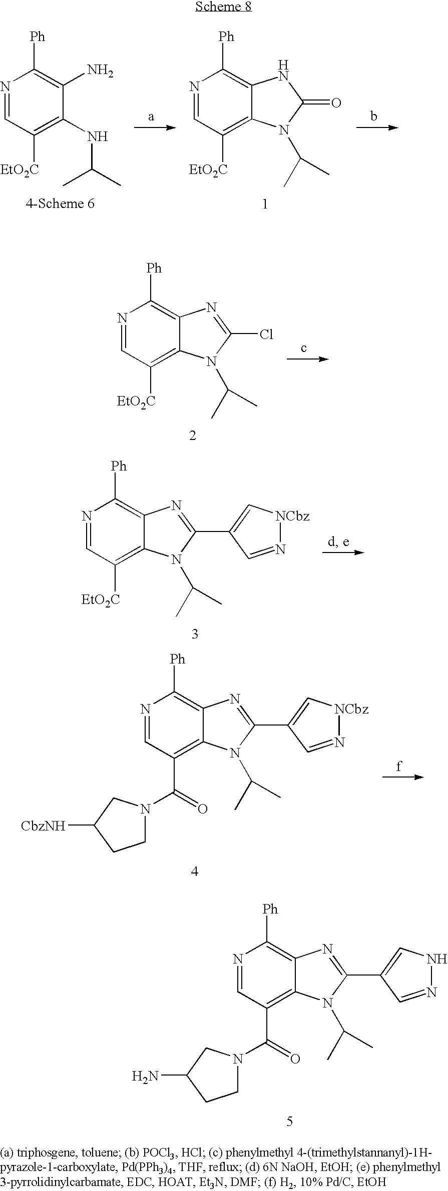

Alternatively, compounds of Formula (I) can be prepared from intermediate 4-Scheme 6. The imidazopyridinone such as 1-Scheme 8 is prepared by condensing a diaminopyridine such as 4-Scheme 6 with a suitable reagent such as triphosgene. Treatment with a halogenating reagent such as POCl₃ gives the corresponding halo-imidazopyridine such as 2-Scheme 8. Reaction with an aryl boronic acid or aryl stannane such as phenylmethyl 4-(trimethylstannanyl)-1H-pyrazole-1-carboxylate in the presence of a catalyst, preferably tetrakistriphenylphosphino palladium in a suitable solvent such as THF gives the corresponding aryl compound such as 3-Scheme 8. Saponification of the ester using a base such as 6N NaOH in a suitable polar solvent such as EtOH gives the corresponding acid. The acid is activated by suitable reagents such as EDC and HOAT in the presence of a suitable base such as Et₃N in a polar solvent such as DMF and condensed with an appropriate amine such as phenylmethyl 3-pyrrolidinylcarbamate to give the corresponding amide such as 4-Scheme 8. The protecting groups are removed under hydrogenolysis conditions using a catalyst such as 10% Pd/C in a suitable solvent such a EtOH to give compounds of Formula (I) such as 5-Scheme 8.

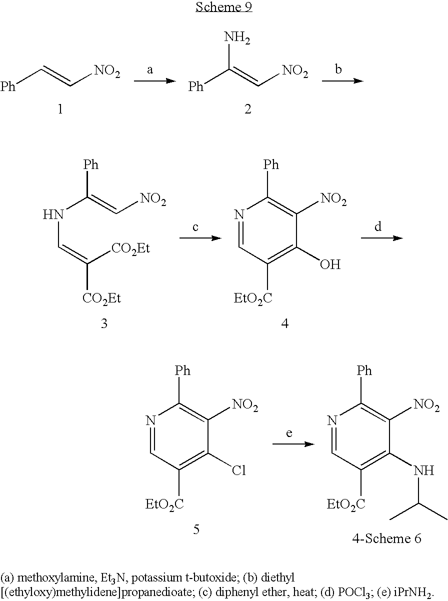

Alternatively, an intermediate like 4-Scheme 6 can be prepared in a manner analogous to those shown in Scheme 9. A suitable nitro-enamine such as 2-Scheme 9 is prepared by condensing a suitable nitroalkene such as 1-Scheme 9 with methoxylamine in the presence of a suitable base such as potassium t-butoxide and Et₃N. A 1,4-addition to diethyl [(ethyloxy)methylidene]propanedioate gives the corresponding enamine such as 3-Scheme 9. Heating in a suitable solvent such as diphenyl ether gives a substituted pyridine such as 4-Scheme 9. Treatment with a chlorination agent such as POCl₃ gives the corresponding pyridyl chloride such as 5-Scheme 9. Treatment with an appropriate primary amine such as i-propyl amine gives an intermediate such as 4-Scheme 6 which can be used to prepare compounds of Formula (I).

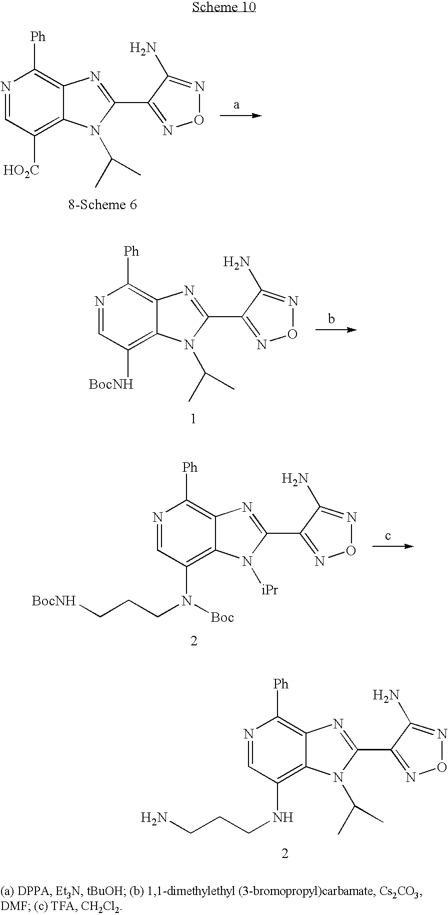

Alternatively, compounds of Formula (I) can be prepared from intermediate 8-Scheme 6. Treatment of the acid with diphenylphosphoryl azide in a suitable solvent such as t-butanol affects a Curtius rearrangement to give a protected amine such as 1-Scheme 10. Other alternative methods exist and are known to those skilled in the art for carrying out this transformation. A compilation of these methods can be found in standard organic synthesis texts such as Hassner and Stumer, “Organic Syntheses Based On Name Reactions and Unnamed Reactions,” Pergamon, N.Y. (1994). Deprotenation with a mild base such as Cs₂CO₃ in a suitable solvent such as DMF followed by alkylation with a suitable alkyl halide such as 1,1-dimethylethyl (3-bromopropyl)carbamate gives the corresponding protected amine such as 2-Scheme 10. The protecting groups are removed using a protic or Lewis acid such as trifluoroacetic acid in a polar solvent such as methylene chloride to give compounds of Formula (I) such as 3-Scheme 10.

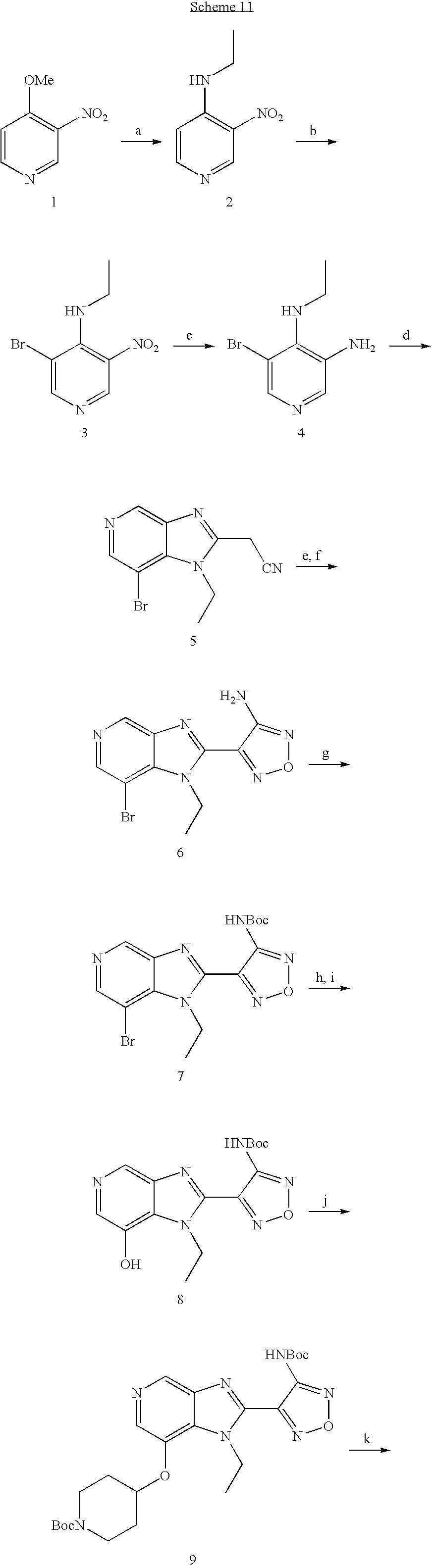

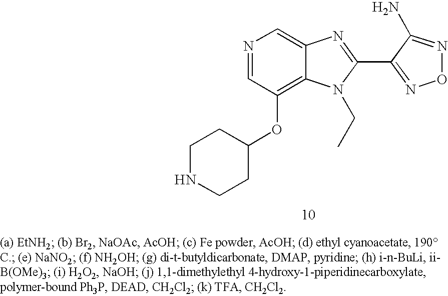

Alternatively, compounds of Formula (I) can be prepared in a manner analogous to that shown in Scheme 11. A suitably substituted pyridine such as 1-Scheme 11 is reacted with a suitable primary amine such as ethyl amine to give the corresponding aminopyridine such as 2-Scheme 11. Bromination of the aminopyridine using bromine buffered in sodium acetate gives the corresponding bromopyridine such as 3-Scheme 11. Reduction of the nitro group can be accomplished using iron powder in acetic acid to give the corresponding diaminopyridine such as 4-Scheme 11. Other alternative methods exist and are known to those skilled in the art for carrying out the previous two transformations. A compilation of these methods can be found in standard organic synthesis texts such as Larock, “Comprehensive Organic Transformations,” VCH, N.Y. (1989). Condensation with ethyl cyanoacetate followed by cyclodehydration upon continued heating gives the corresponding imidazopyridine such as 5-Scheme 11. Reaction with NaNO₂ in concentrated HCl following by reaction with hydroxylamine gives a bis-oxime that cyclodehydrates with continued heating to give an aminofurazan such as 6-Scheme 11. The amino group is protected by reacting with di-t-butyldicarbonate to give the corresponding t-butyl carbamate, 7-Scheme 11. Many different protecting groups are available to one skilled in the art and can be used here as long as they do not interfere with the processes listed herein. The hydroxyl group is introduced by generating an aryl anion by halogen-metal exchange using a suitable base such as n-butyl lithium, reacting the anion with an appropriate boron electrophile such as trimethyl borate and oxidizing the resulting aryl boronate with an appropriate oxidizing agent such as hydrogen peroxide in aqueous base to give imidazopyridinols such as 8-Scheme 11. Etherification of the imidazopyridinol is carried out with an appropriate alcohol such as 11-dimethylethyl 4-hydroxy-1-piperidinecarboxylate using the methods described by Mitsunobu, Synthesis 1981, 1 to give ethers such as 9-Scheme 11. Removal of the protecting groups is achieved using a protic or Lewis acid such as trifluoroacetic acid in a polar solvent such as methylene chloride giving compounds of Formula (I) such as 10-Scheme 11.

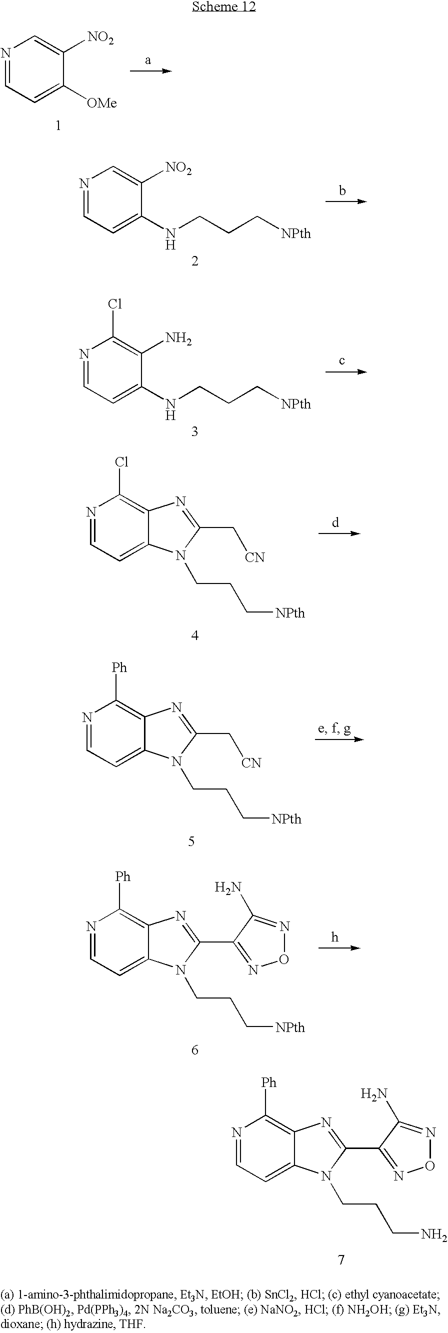

Alternatively, compounds of Formula (I) can be prepared in a manner analogous to those shown in Scheme 12. A suitably substituted pyridine such as 1-Scheme 12 is reacted with a suitable primary amine such as 1-amino-3-phthalimidopropane to give the corresponding aminopyridine such as 2-Scheme 11. Reduction of the nitro group with concomitant introduction of the chloro group is achieved using tin (II) chloride. Condensation with ethyl cyanoacetate followed by cyclodehydration upon continued heating gives the corresponding imidazopyridine such as 4-Scheme 12. Reaction with an aryl boronic acid such as phenylboronic acid in the presence of a catalyst, preferably tetrakistriphenylphosphino palladium and a base such as sodium carbonate or triethylamine in a suitable solvent such as toluene gives the corresponding aryl compound such as 5-Scheme 12. Reaction with NaNO₂ in concentrated HCl following by reaction with hydroxylamine gives a bis-oxime that cyclodehydrates with continued heating to give an aminofurazan such as 6-Scheme 12. Removal of the protecting group is achieved using hydrazine in a suitable solvent such as THF giving compounds of Formula (I) such as 7-Scheme 12.

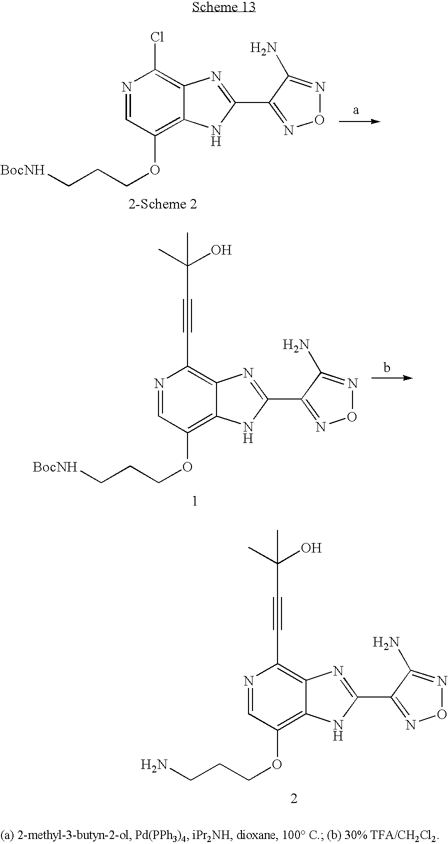

Alternatively, compounds of Formula (I) can be prepared in a manner analogous to that shown in Scheme 13. Treatment of an appropriate aryl halide such as 2-Scheme 2 with an appropriate catalyst such as tetrakistriphenylphosphine palladium and a terminal alkyne in the presence of a suitable base such as di-isopropylamine in an appropriate solvent such as dioxane gives the corresponding aryl alkyne such as 1-Scheme 13. Removal of the protecting groups is achieved using a protic or Lewis acid such as trifluoroacetic acid in a polar solvent such as methylene chloride giving compounds of Formula (I) such as 2-Scheme 13.

In preparing the presently invented compounds of Formula (II), the following novel intermediates are prepared.
4-(7-Bromo-4-chloro-1-ethyl-1H-imidazo[4,5-c]pyridin-2-yl)-1,2,5-oxadiazol-3-amine, is an intermediate that can be prepared as described in Example 18 (e).
2-(4-Amino-1,2,5-oxadiazol-3-yl)-1-methyl-4-phenyl-1H-imidazo[4,5-c]pyridine-7-carboxylic acid, is an intermediate that can be prepared as described in Example 98 (g).

In preparing the presently invented compounds of Formula (I), the following novel intermediate is prepared.

Ethyl 4-chloro-5-nitro-6-phenyl-3-pyridinecarboxylate, is an intermediate that can be prepared as described in Example 98 (d).

The invention also relates to a process for preparing a compound of Formula (I), and/or pharmaceutically acceptable salts, hydrates, solvates and pro-drugs thereof, which comprises converting ethyl 4-chloro-5-nitro-6-phenyl-3-pyridinecarboxylate into a compound of Formula (I), and thereafter optionally preparing a pharmaceutically acceptable salt, hydrate, solvate or pro-drug thereof.

The invention also relates to a process for preparing a compound of Formula (II), and/or pharmaceutically acceptable salts, hydrates, solvates and pro-drugs thereof, which comprises converting 4-(7-Bromo-4-chloro-1-ethyl-1H-imidazo[4,5-c]pyridin-2-yl)-1,2,5-oxadiazol-3-amine into a compound of Formula (II), and thereafter optionally preparing a pharmaceutically acceptable salt, hydrate, solvate or pro-drug thereof.

The invention also relates to a process for preparing a compound of Formula (II), and/or pharmaceutically acceptable salts, hydrates, solvates and pro-drugs thereof, which comprises converting 2-(4-Amino-1,2,5-oxadiazol-3-yl)-1-methyl-4-phenyl-1H-imidazo[4,5-c]pyridine-7-carboxylic acid into a compound of Formula (II), and thereafter optionally preparing a pharmaceutically acceptable salt, hydrate, solvate or pro-drug thereof.

By the term “co-administering” and derivatives thereof as used herein is meant either simultaneous administration or any manner of separate sequential administration of an AKT inhibiting compound, as described herein, and a further active ingredient or ingredients, known to be useful in the treatment of cancer, including chemotherapy and radiation treatment, or to be useful in the treatment of arthritis. The term further active ingredient or ingredients, as used herein, includes any compound or therapeutic agent known to or that demonstrates advantageous properties when administered to a patient in need of treatment for cancer or arthritis. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the presently invented AKT inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β, 10β, 13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guides 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine, N-tert-butyl ester, 13-ester with 5D-20-epoxy-1,2α,4,7β, 10β, 13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H, 12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

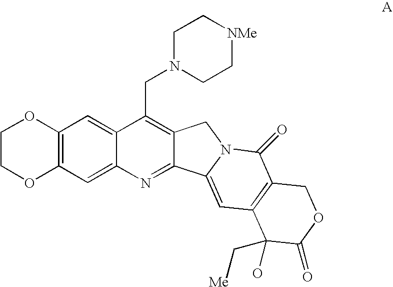

known by the chemical name ″7-(4-methylpiperazino-methylene)-10,11-ethylenedioxy-20(R,S)-camptothecin (racemic mixture) or ″7-(4-methylpiperazino-methylene)-10,11-ethylenedioxy-20(R)-camptothecin (R enantiomer) or ″7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (Trk, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin ® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

Because the pharmaceutically active compounds of the present invention are active as AKT inhibitors they exhibit therapeutic utility in treating cancer and arthritis.

Suitably, the present invention relates to a method for treating or lessening the severity of a cancer selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, pancreatic and prostate.

### Isolation and Purification of His-tagged AKT1 (aa 136-480)

Insect cells expressing His-tagged AKT1 (aa 136-480) were lysed in 25 mM HEPES, 100 mM NaCl, 20 mM imidazole; pH 7.5 using a polytron (5 mLs lysis buffer/g cells). Cell debris was removed by centrifuging at 28,000×g for 30 minutes. The supernatant was filtered through a 4.5-micron filter then loaded onto a nickel-chelating column pre-equilibrated with lysis buffer. The column was washed with 5 column volumes (CV) of lysis buffer then with 5 CV of 20% buffer B, where buffer B is 25 mM HEPES, 100 mM NaCl, 300 mM imidazole; pH 7.5. His-tagged AKT1 (aa 136-480) was eluted with a 20-100% linear gradient of buffer B over 10 CV. His-tagged AKT1 (136-480) eluting fractions were pooled and diluted 3-fold with buffer C, where buffer C is 25 mM HEPES, pH 7.5. The sample was then chromatographed over a Q-Sepharose HP column pre-equilibrated with buffer C. The column was washed with 5 CV of buffer C then step eluted with 5 CV 10% D, 5 CV 20% D, 5 CV 30% D, 5 CV 50% D and 5 CV of 100% D; where buffer D is 25 mM HEPES, 1000 mM NaCl; pH 7.5. His-tagged AKT1 (aa 136-480) containing fractions were pooled and concentrated in a 10-kDa molecular weight cutoff concentrator. His-tagged AKT1 (aa 136-480) was chromatographed over a Superdex 75 gel filtration column pre-equilibrated with 25 mM HEPES, 200 mM NaCl, 1 mM DTT; pH 7.5. His-tagged AKT1 (aa 136-480) fractions were examined using SDS-PAGE and mass spec. The protein was pooled, concentrated and frozen at −80 C.

His-tagged AKT2 (aa 138-481) and His-tagged AKT3 (aa 135-479) were isolated and purified in a similar fashion.

### AKT Enzyme Assay

Compounds of the present invention were tested for AKT 1, 2, and 3 protein serine kinase inhibitory activity in substrate phosphorylation assays. This assay examines the ability of small molecule organic compounds to inhibit the serine phosphorylation of a peptide substrate. The substrate phosphorylation assays use the catalytic domains of AKT 1, 2, or 3. AKT 1, 2 and 3 are also commercially available from Upstate USA, Inc. The method measures the ability of the isolated enzyme to catalyze the transfer of the gamma-phosphate from ATP onto the serine residue of a biotinylated synthetic peptide SEQ. ID NO: 1 (Biotin-ahx-ARKRERAYSFGHHA-amide). Substrate phosphorylation was detected by the following procedure:

Assays were performed in 384 well U-bottom white plates. 10 nM activated AKT enzyme was incubated for 40 minutes at room temperature in an assay volume of 20 ul containing 50 mM MOPS, pH 7.5, 20 mM MgCl₂, 4 uM ATP, 8 uM peptide, 0.04 uCi [g-³³P] ATP/well, 1 mM CHAPS, 2 mM DTT, and 1 ul of test compound in 100% DMSO. The reaction was stopped by the addition of 50 ul SPA bead mix (Dulbecco's PBS without Mg²⁺ and Ca²⁺, 0.1% Triton X-100, 5 mM EDTA, 50 uM ATP, 2.5 mg/ml Streptavidin-coated SPA beads.) The plate was sealed, the beads were allowed to settle overnight, and then the plate was counted in a Packard Topcount Microplate Scintillation Counter (Packard Instrument Co., Meriden, Conn.).

The data for dose responses were plotted as % Control calculated with the data reduction formula 100*(U1-C2)/(C1-C2) versus concentration of compound where U is the unknown value, C1 is the average control value obtained for DMSO, and C2 is the average control value obtained for 0.1M EDTA. Data are fitted to the curve described by: y=((Vmax*x)/(K+x)) where Vmax is the upper asymptote and K is the IC50.

The compound of Example 236 [(S)-3-{3-[2-(4-Amino-furazan-3-yl)-4-(3-chloro-phenyl)-1-ethyl-1H-imidazo[4,5-c]pyridin-7-ylamino]-propylamino}-propane-1,2-diol] demonstrated an IC50 (uM) activity of: 0.069, delta-PH AKT1; 0.038, delta-PH AKT2; and 0.032, delta-PH AKT3 in the above assay.

The pharmaceutically active compounds within the scope of this invention are useful as AKT inhibitors in mammals, particularly humans, in need thereof.

The present invention therefore provides a method of treating cancer, arthritis and other conditions requiring AKT inhibition, which comprises administering an effective compound of Formula (I) or a pharmaceutically acceptable salt, hydrate, solvate or pro-drug thereof. The compounds of Formula (I) also provide for a method of treating the above indicated disease states because of their demonstrated ability to act as Akt inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of an Akt inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular Akt inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing Akt inhibitory activity in mammals, including humans, comprises administering to a subject in need of such activity an effective Akt inhibiting amount of a pharmaceutically active compound of the present invention.

The invention also provides for the use of a compound of Formula (I) in the manufacture of a medicament for use as an Akt inhibitor.

The invention also provides for the use of a compound of Formula (I) in the manufacture of a medicament for use in therapy.

The invention also provides for the use of a compound of Formula (I) in the manufacture of a medicament for use in treating cancer.

The invention also provides for the use of a compound of Formula (I) in the manufacture of a medicament for use in treating arthritis.

The invention also provides for a pharmaceutical composition for use as an Akt inhibitor which comprises a compound of Formula (I) and a pharmaceutically acceptable carrier.

The invention also provides for a pharmaceutical composition for use in the treatment of cancer which comprises a compound of Formula (I) and a pharmaceutically acceptable carrier.

The invention also provides for a pharmaceutical composition for use in treating arthritis which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat cancer or arthritis, or compounds known to have utility when used in combination with an Akt inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

The compounds of Examples 1 to 265 are readily made according to Schemes 1 to 13 or by analogous methods.

### Example 1

### Preparation of 4-(4-Phenyl-1-piperidin-4-yl-1H-imidazo[4,5-c]pyridin-2-yl)-furazan-3-ylamine trifluoroacetate

### a) (1-Benzyl-piperidin-4-yl)-(3-nitro-pyridin-4-yl)-amine

A mixture of 4-methoxy-3-nitropyridine (4.34 g, 28.1 mmol), 4-amino-1-benzypiperidine (6.01 g, 30.9 mmol), and NaOAc (2.31 g, 28.1 mmol) in absolute ethanol (20 mL) was stirred at reflux for 54 h. The reaction mixture was cooled to ambient temperature and concentrated in vacuo. The residue was dissolved in CH₂Cl₂ (100 mL) and washed with water (2×30 mL). The organic layer was dried over anhydrous MgSO₄ and concentrated in vacuo to provide the product (8.78 g) as a dark yellow solid. ¹H NMR (400 MHz, CDCl₃) δ 9.21 (s, 1H), 8.26 (dd, J=6.0, 0.4 Hz, 1H), 8.20 (broad d, J=7.1 Hz, 1H), 7.34-7.25 (complex m, 5H), 6.70 (d, J=6.0 Hz, 1H), 3.62-3.53 (m, 1H), 3.55 (s, 2H), 2.89-2.79 (m, 2H), 2.30-2.20 (m, 2H), 2.10-2.00 (m, 2H), 1.76-1.65 (m, 2H).

### b) N⁴-(1-Benzyl-piperidin-4-yl)-2-chloro-pyridin-3,4-diamine

To a stirred solution of the compound of Example 1(a) (3.00 g, 9.60 mmol) in conc. HCl at 90 C was added tin (II) chloride (9.09 g, 48.0 mmol) portionwise over 10-15 min, at which time the resultant mixture was stirred at 90° C. for additional 30 min. The reaction was cooled to ambient temperature, and the precipitated product (HCl salt thereof) was collected via filtration. The free base was isolated upon treatment of the hydrochloride salt with excess 2.5 N NaOH, followed by an exhaustive extraction with CH₂Cl₂, drying of the combined organic extracts over anhydrous MgSO₄, and solvent evaporation. Additional product can be obtained upon treatment of the HCl filtrate with 50% NaOH solution, followed by removal of the tin salts via filtration, and extraction of the filtrate with CH₂Cl₂. A total of 3.00 g of the product was obtained as a yellow foamy solid. MS (ES+) m/z 317.2 [M+H]⁺.

### c) [1-(1-Benzyl-piperidin-4-yl)-4-chloro-1H-imidazo[4,5-c]pyridin-2-yl]-acetonitrile

A mixture of the compound of Example 1(b) (2.10 g, 6.63 mmol) and ethyl cyanoacetate (5 mL, 46.4 mmol) was heated at 190° C. for 2.5 h. Purification of the crude reaction mixture by flash chromatography (silica gel, 50:1→35:1→20:1 CH₂Cl₂/MeOH gradient) provided the product (1.44 g) as a deep yellow foamy solid.

MS (ES+) m/z 366.2 [M+H]⁺.

### d) [1-(1-Benzyl-piperidin-4-yl)-4-phenyl-1H-imidazo[4,5-c]pyridin-2-yl]-acetonitrile

A solution of the compound of Example 1(c) (185 mg, 0.506 mmol), phenylboronic acid (92 mg, 0.758 mmol), and Pd(PPh₃)₂Cl₂ (35 mg, 0.0506 mmol) in toluene (5 mL) at ambient temperature was treated with a 2 M solution of sodium carbonate, and the resultant dark biphasic mixture was heated at reflux for 3 h. The reaction was cooled to ambient temperature, concentrated in vacuo, and purified by flash chromatography (silica gel, 30:1→10:1 CH₂Cl₂/MeOH gradient) to give the product (177 mg) as a yellow crystalline solid. MS (ES+) m/z 408.2 [M+H]⁺.

### e) [1-(1-Benzyl-piperidin-4-yl)-4-phenyl-1H-imidazo[4,5-c]pyridin-2-yl]-furazan-3-ylamine

To a solution of the compound of Example 1 (d) (165 mg, 0.405 mmol) in MeOH (4 mL) and 2 N HCl (1.5 mL, 3.00 mmol) was added sodium nitrite (56 mg, 0.810 mmol) portionwise. The reaction mixture was stirred at ambient temperature for 1.5 h, at which time the pH of the solution was adjusted to 12 with 50 wt. % NaOH aqueous solution. The resultant dark mixture was then treated with hydroxylamine (50 wt. % solution in water, 1.1 mL, 17.95 mmol) and stirred at 90 C for 15 h. After allowing the reaction to cool to RT, the resulting yellow precipitate was isolated by filtration, washed with cold MeOH and dried under high vacuum to give pure product (85 mg). MS (ES+) m/z 452.2 [M+H]⁺.

### f) 4-(4-Phenyl-1-piperidin-4-yl-1H-imidazo[4,5-c]pyridin-2-yl)-furazan-3-ylamine trifluoroacetate

A solution of the compound of Example 1 (e) (33 mg, 0.073 mmol) in dry CH₂Cl₂ (2.5 mL) at RT was treated with 1-chloroethyl chloroformate (24 μL, 0.219 mmol). The resultant mixture was heated at reflux for 1 h, then cooled to RT and concentrated in vacuo. The residue was then heated at reflux in MeOH for 1 h. The product was isolated by preparative HPLC (Zorbax C18 column, 7 micron particle size, 250 mm×21.2 mm i.d.; 20-90% acetonitrile/water (0.1% TFA); 20 mL/min; UV detection at 254 nm; R_{f}=4.3 min) to afford the product (27 mg) as a white solid. MS (ES+) m/z 362.2 [M+H]⁺.

### Example 2

### Preparation of 4-[4-(3-Chloro-phenyl)-1-piperidin-4-yl-1H-imidazo[4,5-c]pyridin-2-yl-furazan-3-ylamine hydrochloride

The title compound was prepared by substituting 3-chlorophenylboronic acid for phenyl boronic acid in Example 1 (d) and the proceeding as described for Examples 1(e) through 1(f) and triturating with 4N HCl/dioxane. MS (ES+) m/z 396.0 [M+H]⁺.

### Example 3

### Preparation of 4-[1-(3-amino-2,2-dimethylpropyl)-4-(3-chlorophenyl)-1H-imidazo[4,5-c]pyridin-2-yl]-furazan-3-ylamine trifluoroacetate

### a) N¹-(3-Nitropyridin-4-yl)-2,2-dimethyl-1,3-propanediamine

A solution of 4-methoxy-3-nitropyridine (5.00 g, 32.4 mmol) and 2,2-dimethyl-1,3-propanediamine (16.2 g, 161 mmol) in DMF (100 mL) was heated at 100° C. for 5 h. The solvent was removed under reduced pressure to give 7.30 g of the desired compound. ¹H NMR (400 MHz, CDCl₃) δ 9.20 (s, 2H), 9.10 (br, 1H), 8.20 (d, 1H), 6.70 (d, 1H), 3.25 (d, 2H), 2.60 (s, 2H), 1.25 (br, 2H), 0.95 (s, 6H).

### b) 2-[3-(3-Nitropyridin-4-ylamino)-2,2-dimethylpropyl]-isoindole-1,3-dione

A solution of the compound of Example 3(a) (7.30 g, 32.4 mmol) and phthalic anhydride (4.80 g, 32.4 mmol) in glacial acetic acid (160 mL) was heated overnight at 120° C. After 16 h, the solution was allowed to cool to RT and the solvent was removed in vacuo. The residue was partitioned between EtOAc (650 mL) and 5% NaHCO₃ (100 mL). The organic layer was washed with brine (50 mL) and dried over Na₂SO₄. The solvent was removed in vacuo to give 10.5 g of the desired compound. MS (ES) m/z 355.2 [M+H]⁺.

### c) 2-[3-(3-Amino-2-chloropyridin-4-ylamino)-2,2-dimethylpropyl]-isoindole-1,3-dione

A suspension of the compound of Example 3(b) (10.5 g, 29.6 mmol) in conc. HCl (220 mL) was heated to 70° C. and tin (II) chloride dihydrate (35.3 g, 157 mmol) added portionwise. The solution was heated for 30 min at 90° C., allowed to cool and then filtered. The collected solid was partitioned between EtOAc (750 mL) and 0.5N NaOH (200 mL). This mixture was filtered and the filter cake slurried with 1.0N NaOH (75 mL). The slurry was extracted with EtOAc (2×250 mL) and the combined organic layers were washed with brine (70 mL), dried over Na₂SO₄ and concentrated in vacuo to give 5.41 g of the desired compound. MS (ES) m/z 359.2 [M+H]⁺.

### d) 4-Chloro-1-[3-(1,3-dioxo-1,3-dihydroisoindol-2-yl)-2,2-dimethylpropyl]-1H-imidazo[4,5-c]pyridin-2-yl]-acetonitrile

A mixture of the compound of Example 3(c) (5.40 g, 150 mmol) and ethyl cyanoacetate (15 mL) was heated at 190° C. After 6 h, the cooled crude reaction mixture was subjected to flash chromatography (silica gel, Et₂O to 50% Et₂O/CH₂Cl₂) to give 1.70 g of the desired compound. MS (ES) m/z 408.0 [M+H]⁺.

### e) 4-Chloro-1-[3-(1,3-dioxo-1,3-dihydroisoindol-2-yl)-2,2-dimethylpropyl]-1H-imidazo[4,5-c]pyridin-2-yl]-hydroxyiminoacetonitrile

Sodium nitrite (0.15 g, 2.20 mmol) was added to a stirred suspension of the compound of Example 3(d) (0.45 g, 1.10 mmol) in a mixture of MeOH (10 mL) and 2N HCl (4.4 mL). After 18 h, the product was isolated by filtration to give 0.41 g of the desired compound. MS (ES) m/z 437.0 [M+H]⁺.

### f) 4-Chloro-1-[3-(1,3-dioxo-1,3-dihydroisoindol-2-yl)-2,2-dimethylpropyl]-1H-imidazo[4,5-c]pyridin-2-yl]-N-hydroxy-2-hydroxyiminoacetamidine

A solution of the compound of Example 3(e) (0.40 g, 0.92 mmol), Et₃N (1.4 mL) and 50% aqueous hydroxylamine (0.25 mL) in THF (20 mL) was heated in a sealed flask at 90° C. After 1 h, the reaction was allowed to cool to RT and was partitioned between EtOAc (125 mL) and water (50 mL). The organic layer was washed with water (50 mL), brine (40 mL) and dried over Na₂SO₄. The solvent was removed in vacuo to give 0.42 g of the desired compound. MS (ES) m/z 470.2 [M+H]⁺.

### g) 2-{3-[2-(4-Aminofurazan-3-yl)-4-chloro-1H-imidazo[4,5-c]pyridin-1-yl]-2,2-dimethylpropyl}-isoindole-1,3-dione

A solution of the compound of Example 3(f) (0.42 g, 0.91 mmol) in a mixture of dioxane (14 mL) and Et₃N (1.4 mL) was heated to 150° C. in a sealed flask. After 1 h, the reaction was allowed to cool to RT and the solvent was removed in vacuo. Flash chromatography (silica gel, 3% MeOH/CH₂Cl₂) gave 0.32 g of the desired compound. MS (ES) m/z 452.2 [M+H]⁺.

### h) N-{3-[2-(4-Aminofurazan-3-yl)-4-(3-chlorophenyl)-1H-imidazo[4,5-c]pyridin-1-yl]-2,2-dimethylpropyl}-phthalamic acid

A stirred mixture of toluene (5 mL), EtOH (5 mL), 3-chlorophenyl-boronic acid (0.045 g, 0.29 mmol) and the compound of Example 3(g) (0.10 g, 0.22 mmol) was treated with 1.0 M Na₂CO₃ (0.6 mL) followed by (Ph₃P)₄Pd (0.025 g, 0.022 mmol). After 5 h at reflux, the solvent was removed in vacuo and the residue was dissolved in water (5 mL). The solution was adjusted to pH 5 with 0.2 N HCl and the resulting suspension was extracted with EtOAc (3×75 mL). The combined extracts were dried over Na₂SO₄ and the solvent was removed in vacuo. Purification of the by preparative HPLC (10 to 50% acetonitrile/water, 0.1% TFA over 10 min., 50×20 mm. I.D. YMC Combi-Prep ODS-A) gave 0.068 g of the desired compound. MS (ES) 546.2 [M+H]⁺.

### i) 4-[1-(3-Amino-2,2-dimethylpropyl)-4-(3-chlorophenyl)-1H-imidazo[4,5-c]pyridin-2-yl]-furazan-3-ylamine trifluoroacetate

A solution of the compound of Example 3(h) (0.055 g, 0.083 mmol) in a mixture of EtOH (7 mL) and hydrazine hydrate (3 mL) was heated at reflux for 20 h. The solvent was removed in vacuo and the residue subjected to preparative HPLC (10 to 50% acetonitrile/water, 0.1% TFA over 10 min., 50×20 mm. I.D. YMC Combi-Prep ODS-A) to give 0.020 g of the title compound. MS (ES) m/z 398.2 [M+H]⁺.

### Example 4

### Preparation of 4-[1-(3-amino-2,2-dimethylpropyl)-4-Phenyl-1H-imidazo[4,5-c]pyridinyl-2-yl]-furazan-3-ylamine trifluoroacetate

The title compound was prepared in an analogous manner to Example 3 by substituting phenyl boronic acid for 3-chlorophenyl-boronic acid in step (h). MS (ES) m/z 364.2 [M+H]⁺.

### Example 5

### Preparation of 4-[1-(5-aminopentyl)-4-phenyl-1H-imidazo[4,5-c]pyridin-2-yl]-1,2,5-oxadiazol-3-amine trifluoroacetate

The title compound was prepared in an analogous manner to Example 3 by substituting 1,5-diaminopentane for 2,2-dimethyl-1,3-propanediamine in step (a) and phenyl boronic acid for 3-chlorophenyl-boronic acid in step (h). MS (ES) m/z 364.0 [M+H]⁺.

### Example 6

### Preparation of 4-[1-(6-aminohexyl)-4-phenyl-1H-imidazo[4,5-c]pyridin-2-yl]-1,2,5-oxadiazol-3-amine trifluoroacetate

The title compound was prepared in an analogous manner to Example 3 by substituting 1,6-diaminohexane for 2,2-dimethyl-1,3-propanediamine in step (a) and phenyl boronic acid for 3-chlorophenyl-boronic acid in step (h). MS (ES) m/z 378.0 [M+H]⁺.

### Example 7

### Preparation of 4-[1-(5-aminopentyl)-4-(3-chlorophenyl)-1H-imidazo[4,5-c]pyridin-2-yl]-1,2,5-oxadiazol-3-amine trifluoroacetate

The title compound was prepared in an analogous manner to Example 3 by substituting 1,5-diaminopentane for 2,2-dimethyl-1,3-propanediamine in step (a).

MS (ES) m/z 398.0 [M+H]⁺.

### Example 8

### Preparation of 4-[1-(6-aminohexyl)-4-(3-chlorophenyl)-1H-imidazo[4,5-c]pyridin-2-yl]-1,2,5-oxadiazol-3-amine trifluoroacetate

The title compound was prepared in an analogous manner to Example 3 by substituting 1,6-diaminohexane for 2,2-dimethyl-1,3-propanediamine in step (a).

MS (ES) m/z 412.0 [M+H]⁺.

### Example 9

### Preparation of 4-[1-(3-amino-2,2-dimethylpropyl)-4-(3-methoxyphenyl)-1H-imidazo[4,5-c]pyridinyl-2-yl]-furazan-3-ylamine trifluoroacetate

The title compound was prepared in an analogous manner to Example 3 by substituting 3-methoxyphenyl boronic acid for 3-chlorophenyl-boronic acid in step (h). MS (ES) m/z 394.2 [M+H]⁺.

### Example 10

### Preparation of 4-[1-(5-aminopentyl)-4-(3-thienyl)-1H-imidazo[4,5-c]pyridin-2-yl]-1,2,5-oxadiazol-3-amine trifluoroacetate

The title compound was prepared in an analogous manner to Example 3 by substituting 1,5-diaminopentane for 2,2-dimethyl-1,3-propanediamine in step (a) and 3-thienylboronic acid for 3-chlorophenyl-boronic acid in step (h). MS (ES) m/z 370.0 [M+H]⁺.

### Example 11

### Preparation of 4-[1-(6-aminohexyl)-4-(3-thienyl)-1H-imidazo[4,5-c]pyridin-2-yl]-1,2,5-oxadiazol-3-amine trifluoroacetate

The title compound was prepared in an analogous manner to Example 3 by substituting 1,6-diaminohexane for 2,2-dimethyl-1,3-propanediamine in step (a) and 3-thienylboronic acid for 3-chlorophenyl-boronic acid in step (h). MS (ES) m/z 384.0 [M+H]⁺.

### Example 12

### Preparation of 4-[4-chloro-1-(cyclopropylmethyl)-1H-imidazo[4,5-c]pyridin-2-yl]-1,2,5-oxadiazol-3-amine

### a) N-(Cyclopropylmethyl)-3-nitro-4-pyridinamine

4-methoxy-3-nitropyridine (10.0 g, 64 mmol), cyclopropylmethyl amine (4.56 g, 64 mmol), and EtOH (7 mL) were combined in a sealed tube and heated to 85° C. with vigourous shaking for 48 h. The mixture was concentrated in vacuo to afford the desired compound as a solid (12.0 g). MS (ES+) m/z 194 [M+H]⁺.

### b) 2-Chloro-N⁴-(cyclopropylmethyl)-3,4-pyridinediamine

A solution of the compound of Example 12(a) (12.0 g, 62 mmol) in EtOH (136 mL) was cooled to 0° C. Conc. HCl (136 mL) was added and the mixture was stirred at 0° C. for 15 min. Tin (II) chloride dihydrate (42.2 g, 188 mmol) was added and stirring was continued at 0° C. for 3 h. The reaction was quenched by adjusting to pH 8 with 1 M NaOH. The mixture was extracted with EtOAc (200 mL×3) and the combined extracts were washed with brine (300 mL), dried over Na₂SO₄ and concentrated in vacuo to afford the desired compound (3.98 g). MS (ES+) m/z 198 [M+H]⁺.

### c) [4-Chloro-1-(cyclopropylmethyl)-1H-imidazo[4,5-c]pyridin-2-yl]acetonitrile

The compound of Example 12(b) (3.98 g, 20 mmol), ethylcyanoacetate (10 mL, 94 mmol), and N,N-dimethylacetamide (10 mL) were combined in a sealed tube and heated to 150° C. for 3 h. The mixture was cooled to RT and concentrated in vacuo. Flash chromatography (silica gel, MeOH/CHCl₃ gradient) yielded the desired compound (3.83 g). MS (ES+) m/z 247 [M+H]⁺.

### d) (2E)-[4-Chloro-1-(cyclopropylmethyl)-1H-imidazo[4,5-c]pyridin-2-yl](hydroxyimino)ethanenitrile

Sodium nitrite (2.11 g, 31 mmol) was added to a solution of the compound of Example 12(c) (3.83 g, 16 mmol) in MeOH (110 mL) and 2M HCl (50 mL). The mixture was stirred at RT for 1.5 h and then cooled to 0° C. The resulting precipitate was collected via filtration, rinsed with cold water and dried to afford the desired compound as a yellow solid (2.4 g). MS (ES+) m/z 276 [M+H]⁺.

### e) 4-[4-Chloro-1-(cyclopropylmethyl)-1H-imidazo[4,5-c]pyridin-2-yl]-1,2,5-oxadiazol-3-amine

The compound of Example 12(d) (2.4 g, 8.7 mmol), THF (58 mL), Et₃N (4.7 mL), and 50% aqueous hydroxylamine (1.56 mL) were combined in a sealed tube and heated to 100° C. for 48 h. The mixture was then cooled to RT and concentrated in vacuo. Flash chromatography (silica gel, MeOH/CHCl₃ gradient) yielded the title compound (1.6 g). MS (ES+) m/z 291 [M+H]⁺.

### Example 13

### Preparation of 4-[4-(3-chlorophenyl)-1-(cyclopropylmethyl)-1H-imidazo[4,5-c]pyridin-2-yl]-1,2,5-oxadiazol-3-amine

A mixture of toluene (8.4 mL) and 2M Na₂CO₃ (1.0 mL) was deoxygenated by purging with nitrogen. The compound of Example 12(e) (100 mg, 0.31 mmol), 3-chlorophenyl boronic acid (81 mg, 0.52 mmol), and dichlorobis(triphenylphosphine)palladium(II) (24 mg, 0.035 mmol) were added and the mixture was heated to 100° C. for 16 h. After cooling to RT, the reaction was concentrated in vacuo. Flash chromatography (silica gel, MeOH/CHCl₃ gradient) gave the title compound (66 mg). MS (ES+) m/e 367 [M+H]⁺.

### Example 14

### Preparation of 4-[1-(cyclopropylmethyl)-4-(2-methylphenyl)-1H-imidazo[4,5-c]pyridin-2-yl]-1,2,5-oxadiazol-3-amine

The title compound was prepared in an analogous manner to Example 13 by substituting 2-methylphenylboronic acid for 3-chlorophenylboronic acid.

MS (ES+) m/z 347.0 [M+H]⁺.

### Example 15

### Preparation of 4-[4-(2-chlorophenyl)-1-(cyclopropylmethyl)-1H-imidazo[4,5-c]pyridin-2-yl]-1,2,5-oxadiazol-3-amine

The title compound was prepared in an analogous manner to Example 13 by substituting 2-chlorophenylboronic acid for 3-chlorophenylboronic acid.

MS (ES+) m/z 367.0 [M+H]⁺.

### Example 16

### Preparation of 4-[1-(cyclopropylmethyl)-4-(3-furanyl)-1H-imidazo[4,5-c]pyridin-2-yl]-1,2,5-oxadiazol-3-amine

The title compound was prepared in an analogous manner to Example 13 by substituting 3-furanylboronic acid for 3-chlorophenyl boronic acid. MS (ES+) m/z 323.0 [M+H]⁺.

### Example 17

### Preparation of 4-[1-ethyl-7-(Piperidin-4-yloxy)-1H-imidazo[4,5-c]pyridin-2-yl]-furazan-3-ylamine trifluoroacetate

### a) Ethyl (3-nitropyridin-4-yl)amine

A solution consisting of 4-methoxy-3-nitropyridine (15.0 g, 97.3 mmol) with ethyl amine (46.5 mL of 70% aqueous solution, 584 mmol) in ethanol (30 mL) was stirred at 85° C. in a sealed flask for 2 h. Removal of all volatiles in vacuo afforded the title compound (16.2 g, 99%). MS: (M+H)+=m/z 168.

### b) Ethyl (3-bromo-5-nitropyridin-4-yl)amine

A mixture consisting of ethyl (3-nitropyridin-4-yl)amine (11.76 g, 70 mmol) in acetic acid (140 mL) with sodium acetate (28.7 g, 350 mmol) and bromine (13.44 g, 84 mmol) was stirred in a sealed flask at 100° C. for 18 h. Most of the solvent was removed in vacuo and the residue partitioned between CH₂Cl₂ and water and the aqueous layer basified with NaHCO₃. The organic extract was washed with water then brine, dried (Na₂SO₄) and all volatiles removed in vacuo. The residue was chromatographed on silica gel eluted with ethyl acetate:hexane (2:8) to afford the title compound (10.4 g, 60%). MS: (M+H)⁺=m/z 246.

### c) 5-Bromo-N⁴-ethyl-pyridine-3,4-diamine

A mixture of ethyl (3-bromo-5-nitropyridin-4-yl)amine (7.0 g, 28.4 mmol) in acetic acid (100 mL) with iron powder (<50 micron, 9.51 g, 170 mmol) was stirred at 75° C. for 1 h. The reaction mixture was cooled then diluted with EtOAc:CH₂Cl₂ (1:4) and filtered through celite. The filtrate was concentrated in vacuo then chromatographed on silica gel eluted with ethyl acetate:methanol (96:4) to afford the title compound (5.68 g, 92.7%). MS: (M+H)⁺=m/z 216.

### d) (7-Bromo-1-ethyl-1H-imidazo[4,5-c]pyridin-2-yl)-acetonitrile

A solution of 5-Bromo-N⁴-ethyl-pyridine-3,4-diamine (5.68 g, 26.3 mmol) in ethyl cyanoacetate (5.6 mL, 52.6 mmol) was stirred at 190° C. for 1 h. The product crystallized on cooling and addition of EtOAc (50 mL). The solid was collected, washed with EtOAc then dried to afford the title compound (4.15 g, 59%). MS: (M+H)⁺=m/z 265.

### e) 4-(7-Bromo-1-ethyl-1H-imidazo[4,5-c]pyridin-2-yl)-[1,2,5]oxadiazolidin-3-ylamine

To a solution of (7-bromo-1-ethyl-1H-imidazo[4,5-c]pyridin-2-yl)-acetonitrile (3.2 g, 12.1 mmol) in methanol (40 mL) was added in portions sodium nitrite (1.67 g, 24.2 mmol). The resulting mixture was stirred 2 h then adjusted to pH 12 with 50% aqueous NaOH. To this was added 50% aqueous NH₂OH (33 mL) and the mixture was stirred at 90° C. for 2 h. The solid which formed on cooling was collected by filtration to afford the title compound (2.50 g, 67%). MS: (M+H)⁺=m/z 309.

### f) [4-(7-Bromo-1-ethyl-1H-imidazo[4,5-c]pyridin-2-yl)-furazan-3-yl]-carbamic acid tert-butyl ester

A solution consisting of 4-(7-bromo-1-ethyl-1H-imidazo[4,5-c]pyridin-2-yl)-[1,2,5]oxadiazolidin-3-ylamine (2.14 g, 6.95 mmol) in methylene chloride (10 mL) and pyridine (20 mL) with di-t-butyl dicarbonate (2.27 g, 10.43 mmol) and DMAP (0.85 g, 6.95 mmol) was stirred at 90° C. in a sealed tube for 2.5 h. Additional di-t-butyl dicarbonate (2.27 g, 10.43 mmol) was added and stirring at 90° C. continued for 18 h. The product mixture was partitioned between EtOAc and water, the layers separated and the organic extract washed with water then brine, dried (Na₂SO₄) and all volitiles removed in vacuo. The residue was chromatographed on silica 20% EtOAc in hexane to afford the title compound as an off-white solid 1.60 g, 58.4%) MS: (M+H)⁺=m/z 409.

### g) [4-(1-Ethyl-7-hydroxy-1H-imidazo[4,5-c]pyridin-2-yl)-furazan-3-yl]-carbamic acid tert-butyl ester

To a solution of [4-(7-bromo-1-ethyl-1H-imidazo[4,5-c]pyridin-2-yl)-furazan-3-yl]-carbamic acid tert-butyl ester (205 mg, 0.5 mmol) in THF (4 mL) stirred at −78° C. under N₂ was added n-BuLi (0.5 mL of 2.5 M solution in hexane, 1.25 mmol). This was stirred at −78° C. for 20 min then trimethyl borate (168 uL, 1.5 mmol) with THF (1 mL) was added. Stirring was continued for 1.5 h while the reaction mixture was allowed to warm to room temperature. To the resulting mixture was added a solution consisting of 30% H₂O₂ (1.1 mL) in 3N NaOH (0.35 mL) and stirring continued at room temperature for 30 min. The reaction mixture was diluted with EtOAc then washed with 1N NaOH (3×). The combined aqueous extract was acidified with 6N HCl and the product extracted into EtOAc. The organic extract was dried (Na₂SO₄) and all volitiles removed in vacuo to afford the product as an orange solid (144 mg, 83%). MS: (M+H)⁺=m/z 347.

### h) 4-[2-(4-tert-Butoxycarbonylamino-furazan-3-yl)-1-ethyl-1H-imidazo[4,5-c ]pyridin-7-yloxy]-piperidine-1-carboxylic acid tert-butyl ester

To a stirred mixture of triphenyl phosphine polystyrene (2.4 g, 1.2 mmol/g, 2.88 mmol) in CH₂Cl₂ (25 mL) was added 4-hydroxypiperidine-1-carboxylic acid tert-butyl ester (1.15 g, 5.76 mmol) followed by diethyl azodicarboxylate (0.45 mL, 2.88 mmol). After 10 min at room temperature the mixture was cooled to 0° C. and a solution of [4-(1-ethyl-7-hydroxy-1H-imidazo[4,5-c]pyridin-2-yl)-furazan-3-yl]-carbamic acid tert-butyl ester (200 mg, 0.58 mmol) in CH₂Cl₂ (15 mL) was added. This was stirred 1.5 h at 0° C. then filtered. the resin was washed with CH₂Cl₂ and the combined organic extract washed with 1 N NaOH soln then water, dried (Na₂SO₄) and all volitiles removed. The residue was purified by preparative HPLC (eluted with CH₃CN/H₂O/0.1% TFA) to afford the title compound as an off white solid (131 mg, 43%). MS: (M+H)⁺=m/z 530.

### i) 4-[1-Ethyl-7-(piperidin-4-yloxy)-1H-imidazo[4,5-c]pyridin-2-yl]-furazan-3-ylamine trifluoroacetate

A solution of 4-[2-(4-tert-butoxycarbonylamino-furazan-3-yl)-1-ethyl-1H-imidazo[4,5-c]pyridin-7-yloxy]-piperidine-1-carboxylic acid tert-butyl ester (130 mg, 0.25 mmol) in CH₂Cl₂ (1.5 mL) with TFA (0.75 mL) was stirred at room temperature for 40 min. Removal of all volatiles followed by purification by preparative HPLC (eluted with CH₃CN/H₂O) afforded the title compound (80 mg, 97%). MS: (M+H)⁺=m/z 330.

### Example 18

### Preparation of 1-[2-(4-Amino-furazan-3-yl)-1-ethyl-4-(3-chloro-phenyl)-1H-imidazo[4,5-c]pyridin-7-yl]-1-(3-amino-pyrrolidin-1-yl)-methanone trifluoroacetate

### a) 5-Bromo-2-chloro-N⁴-ethyl-pyridine-3,4-diamine

To a solution of ethyl (3-bromo-5-nitropyridin-4-yl)amine (22.0 g, 89.4 mmol) in concentrated HCl (250 mL) was added in portions tin(II) chloride dihydrate (60.5 g, 270 mmol). The mixture was stirred 1 h at room temperature then poured into ice (300 g). EtOAc (500 mL) was added and the mixture made basic with NaOH. The layers were separated and the organic extract washed with water then brine, dried (Na₂SO₄) and all volatiles removed. The residue was purified by chromatography on silica eluted with 25% EtOAc, 75% hexanes to afford the title compound (17.8 g, 80%). MS (ES+) m/z 250 (M+H)⁺.

### b) N-(5-Bromo-2-chloro-4-ethylamino-pyridin-3-yl)-cyanoacetamide

To a solution of 5-bromo-2-chloro-N⁴-ethyl-pyridine-3,4-diamine (17.7 g, 70.9 mmol) in DMF (100 mL) stirred at 0° C. was added cyanoacetic acid (9.06 g, 106 mmol), N-methyl morpholine (39 mL, 350 mmol) and EDCI (20.3 g, 106 mmol). The cooling bath was removed and stirring continued 3 h. EtOAc (300 mL) was added and the resulting mixture was washed with water then brine. crystallization from EtOAc/hexanes afforded the title compound (22.5 g, quantative). MS (ES+) m/z 317 (M+H)⁺.

### c) (7-Bromo-4-chloro-1-ethyl-1H-imidazo[4,5-c]pyridin-2-yl)-acetonitrile

A solution of N-(5-bromo-2-chloro-4-ethylamino-pyridin-3-yl)-cyanoacetamide (35.6 g, 112 mmol) in acetic acid (300 mL) was stirred at 90° C. for 1 h. All volatiles were removed to afford the title compound used as is in the next step (29.5 g). MS (ES+) m/z 299 (M+H)⁺.

### d) (7-Bromo-4-chloro-1-ethyl-1H-imidazo[4,5-c]pyridin-2-yl)-hydroxyimino-acetonitrile

To a mixture of (7-bromo-4-chloro-1-ethyl-1H-imidazo[4,5-c]pyridin-2-yl)-acetonitrile (29.5 g, 98 mmol) in 2 N HCl (400 mL) was added portion wise, at room temperature, over 20 min sodium nitrite (14.0 g, 203 mmol). After stirring an additional 30 min the precipitated product was filtered, washed with water and dried to afford the title compound used as is in the next step (32 g, quant.). MS (ES+) m/z 328 (M+H)⁺.

### e) 4-(7-Bromo-4-chloro-1-ethyl-1H-imidazo[4,5-c]pyridin-2-yl)-1,2,5-oxadiazol-3-amine

A solution of (7-bromo-4-chloro-1-ethyl-1H-imidazo[4,5-c]pyridin-2-yl)-hydroxyimino-acetonitrile (98 mmol crude from previous step) in THF (250 mL) with TEA (40 mL) and 50% hydroxylamine in water (16 mL) was stirred in a sealed flask at 90° C. for 1.5 h. The solution was cooled to room temperature then partitioned between EtOAc and water. The organic extract was washed with brine, dried and all volatiles removed. the residue was dissolved in dioxane (200 mL) with TEA (35 mL) and stirred in a sealed flask at 150° C. for 1.5 h. The solvent was removed in vacuo and the residue crystallized from methylene chloride to afford the title compound (17.3 g, 51% for three steps). MS (ES+) m/z 343 (M+H)⁺.

### f) 1,1-Dimethylethyl [4-(7-bromo-4-chloro-1-ethyl-1H-imidazo[4,5-c]pyridin-2-yl)-1,2,5-oxadiazol-3-yl]carbamate

A solution consisting of 4-(7-bromo-4-chloro-1-ethyl-1H-imidazo[4,5-c]pyridin-2-yl)-1,2,5-oxadiazol-3-amine (8.5 g, 24.7 mmol) in 1,2-dichloroethane (140 mL) and pyridine (70 mL) with di-t-butyl dicarbonate (21.54 g, 98.8 mmol) and DMAP (3.01 g, 24.7 mmol) was stirred at 85° C. in a sealed flask for 1 h. The product mixture was partitioned between EtOAc and 1N HCl, the layers separated and the organic extract washed with 1N HCl then brine, dried (Na₂SO₄) and all volatiles removed in vacuo. The residue was triturated with EtOAc to afford the title compound as beige solid (5.06 g), MS (ES+) m/z 443 (M+H)⁺. The mother liquor was evaporated to dryness and treated with 2% trifluoroacetic acid in dichloromethane (100 mL) for 20 h. The reaction mixture was neutralized with saturated sodium bicarbonate, then washed with brine, dried (Na₂SO₄) and all volatiles removed in vacuo The residue was chromatographed on silica (20% EtOAc in hexane) to afford the title compound (2.45 g). MS (ES+) m/z 443 (M+H)⁺. The combined weight of the title compound was 8.55 g (78%).

### g) 4-Chloro-2-[4-({[(1,1-dimethylethyl)oxy]carbonyl}amino)-1,2,5-oxadiazol-3-yl]-1-ethyl-1H-imidazo[4,5-c]pyridine-7-carboxylic acid

To a solution of [4-(7-bromo-4-chloro-1-ethyl-1H-imidazo[4,5-c]pyridin-2-yl)furazan-3-yl]carbamic acid tert-butyl ester (1.0 g, 2.25 mmol) in dry THF stirred at −78° C. under N₂ was added n-butyl lithium (2.7 mL of 2.5 M solution in hexanes, 6.75 mmol) rapidly dropwise. This was stirred 1 min then CO₂ was bubbled through the solution for 30 min while the temperature was maintained at −78° C. The mixture was allowed to warm to room temperature then partitioned between EtOAc and 1 N HCl. The organic extract was washed with water then brine and dried (Na₂SO₄). The organic solution was passed through a silica plug then all volatiles were removed in vacuo to afford the title compound (620 mg, 67%). MS: (M+H)⁺=m/z 409.

### h) (4-{7-[1-(3-tert-Butoxycarbonylaminopyrrolidin-1-yl)methanoyl]-4-chloro-1-ethyl-1H-imidazo[4,5-a]pyridin-2-yl}furazan-3-yl)carbamic acid tert-butyl ester

A mixture consisting of 4-chloro-2-[4-({[(1,1-dimethylethyl)oxy]carbonyl}amino)-1,2,5-oxadiazol-3-yl]-1-ethyl-1H-imidazo[4,5-c]pyridine-7-carboxylic acid (410 mg, 1 mmol), Pyrrolidin-3-yl-carbamic acid tert-butyl ester (327 mg, 2 mmol), HOAT (272 mg, 2 mmol), EDCI (383 mg, 2 mmol) and N-methyl morpholine (2 mL) in DMF (4 mL) was stirred at room temperature for 20 h. The mixture was partitioned between EtOAc and 1 N HCl. The organic extract was washed with water then brine, dried (Na₂SO₄) and all volitiles removed in vacuo. Chromatography on silica (eluted with 75% EtOAc, 25% hexanes) afforded the title compound (375 mg, 81%). MS: (M+H)⁺=m/z 577.

### i) {4-[7-(3-tert-Butoxycarbonylaminopyrrolidin-1-ylmethyl)-4-(3-chlorophenyl)-1-ethyl-1H-imidazo[4,5-c]pyridin-2-yl]furazan-3-yl}carbamic acid tert-butyl ester

A mixture consisting of (4-{7-[1-(3-tert-Butoxycarbonylaminopyrrolidin-1-yl)methanoyl]-4-chloro-1-ethyl-1H-imidazo[4,5-a]pyridin-2-yl}furazan-3-yl)carbamic acid tert-butyl ester (100 mg, 0.17 mmol), 3-chlorophenylboronic acid (53 mg, 0.34 mmol) and tetrakis(triphenylphosphine)palladium(0) (25 mg) in toluene (2.3 mL) with EtOH (0.25 mL) and 2 M aqueous Na₂CO₃ solution (0.30 mL) was stirred at 90° C. for 18 h in a sealed tube. The organic solution was separated and chromatographed on silica (eluted with 60% EtOAc, 40% hexanes) to afford the title compound (130 mg, 86%). MS: (M+H)⁺=m/z 653.

### j) 1-[2-(4-Amino-furazan-3-yl)-1-ethyl-4-(3-chlorophenyl)-1H-imidazo[4,5-c]pyridin-7-yl]-1-(3-amino-pyrrolidin-1-yl)-methanone trifluoroacetate

A solution of {4-[7-((S)-3-tert-butoxycarbonylaminopyrrolidin-1-ylmethyl)-4-(3-chlorophenyl)-1-ethyl-1H-imidazo[4,5-c]pyridin-2-yl]furazan-3-yl}carbamic acid tert-butyl ester (130 mg, 0.2 mmol) in CH₂Cl₂ (2 mL) with TFA (1 mL) was stirred at room temperature for 1 h. All volatiles were removed and the residue purified by HPLC (acetonitrile water gradient 0.1% TFA) to afford the title compound (61 mg, 68%). MS: (M+H)⁺=m/z 453.

### Example 19

### Preparation of 1-[2-(4-Aminofurazan-3-yl)-1-ethyl-4-phenyl-1-H-imidazo[4,5-c]pyridin-7-yl-1-(3-aminopyrrolidin-v-yl)methanone hydrochloride

The title compound was prepared in an analogous manner to Example 18 by substituting phenyl boronic acid for 3-chlorophenylboronic acid in step (i) and triturating with 4N HCl/dioxane. MS (ES+) m/z 419.0 [M+H]⁺.

### Example 20

### Preparation of 1-[2-(4-Aminofurazan-3-yl)-1-ethyl-4-thiophen-3-yl-1-H-imidazo[4,5-c]pyridin-7-yl]-1-(3-aminopyrrolidin-1-yl)methanone hydrochloride

The title compound was prepared in an analogous manner to Example 18 by substituting 3-thienylboronic acid for 3-chlorophenylboronic acid in step (i) and triturating with 4N HCl/dioxane. MS (ES+) m/z 425.0 [M+H]⁺.

### Example 21

### Preparation of 1-[2-(4-Aminofurazan-3-yl)-1-ethyl-4-pyridin-yl-1-H-imidazo[4,5-c]pyridin-7-yl]-1-(3-aminopyrrolidin-1-yl)methanone

The title compound was prepared in an analogous manner to Example 18 by substituting 4-pyridylboronic acid for 3-chlorophenylboronic acid in step (i).

MS (ES+) m/z 420.0 [M+H]⁺.

### Example 22

### Preparation of 1-[2-(4-Aminofurazan-3-yl)-1-ethyl-4-pyridin-3-yl-1-H-imidazo[4,5-c]pyridin-7-yl]-1-(3-aminopyrrolidin-1-yl)methanone

The title compound was prepared in an analogous manner to Example 18 by substituting 3-pyridylboronic acid for 3-chlorophenylboronic acid in step (i).

MS (ES+) m/z 420.0 [M+H]⁺.

### Example 23

### Preparation of 1-[2-(4-Aminofurazan-3-yl)-1-ethyl-4-furan-3-yl-1-H-imidazo[4.5-c]pyridin-7-yl]-1-(3-aminopyrrolidin-1-yl)methanone

The title compound was prepared in an analogous manner to Example 18 by substituting 3-furanylboronic acid for 3-chlorophenylboronic acid in step (i).

MS (ES+) m/z 409.0 [M+H]⁺.

### Example 24

### Preparation of 1-[2-(4-Amino-furazan-3-yl)-4-chloro-1-ethyl-1-H-imidazo[4,5-c]pyridin-7-yl]-1-(3-amino-pyrrolidin-1-yl)-methanone trifluoroacetate

The title compound was prepared in an analogous manner to Example 18 except omitting step (i). MS (ES+) m/z 409.0 [M+H]⁺.

### Example 25

### Preparation of 1-[2-(4-Amino-furazan-3-yl)-4-(1H-pyrrol-2-yl))-1-ethyl-1-H-imidazo[4,5-c]pyridin-7-yl]-1-(3-amino-pyrrolidin-1-yl)-methanone trifluoroacetate

The title compound was prepared in an analogous manner to Example 18 by substituting 2-pyrroleboronic acid for 3-chlorophenylboronic acid in step (i).

MS (ES+) m/z 408.0 [M+H]⁺.

### Example 26

### Preparation of 1-[2-(4-Amino-furazan-3-yl)-1-ethyl-4-(2-methoxyphenyl)-1H-imidazo[4,5-c]pyridin-7-yl]-1-(3-amino-pyrrolidin-1-yl)-methanone trifluoroacetate

The title compound was prepared in an analogous manner to Example 18 by substituting 2-methoxyphenylboronic acid for 3-chlorophenylboronic acid in step (i). MS (ES+) m/z 449.0 [M+H]⁺.

### Example 27

### Preparation of 1-[2-(4-Amino-furazan-3-yl)-1-ethyl-4-furan-2-yl-1H-imidazo[4,5-c]pyridin-7-yl]-1-(3-amino-pyrrolidin-1-yl)-methanone trifluoroacetate

The title compound was prepared in an analogous manner to Example 18 by substituting 2-furanylboronic acid for 3-chlorophenylboronic acid in step (i).

MS (ES+) m/z 409.0 [M+H]⁺.

### Example 28

### Preparation of 2-(4-Amino-furazan-3-yl)-1-ethyl-4-phenyl-1H-imidazo[4,5-c]pyridine-7-carboxylic acid [1-(4-chloro-benzyl)-2-hydroxy-ethyl]-amide

The title compound was prepared in an analogous manner to Example 18 by substituting 2-amino-3-(4-chlorophenyl)-1-propanol for pyrrolidin-3-yl-carbamic acid tert-butyl ester in step (h) and phenylboronic acid for 3-chlorophenylboronic acid in step (i). MS (ES+) m/z 518.0 [M+H]⁺.

### Example 29

### Preparation of 2-(4-Amino-furazan-3-yl)-1-ethyl-4-(3-chloro-phenyl)-1H-imidazo[4,5-c]pyridine-7-carboxylic acid [1-(4-chloro-benzyl)-2-hydroxy-ethyl]-amide

The title compound was prepared in an analogous manner to Example 18 by substituting 2-amino-3-(4-chlorophenyl)-1-propanol for pyrrolidin-3-yl-carbamic acid tert-butyl ester in step (h). MS (ES+) m/z 552.0 [M+H]⁺.

### Example 30

### Preparation of 2-(4-Amino-furazan-3-yl)-1-ethyl-4-(2,3-dichloro-phenyl)-1H-imidazo[4,5-c]pyridine-7-carboxylic acid [1-(4-chloro-benzyl)-2-hydroxy-ethyl]-amide trifluoroacetate

The title compound was prepared in an analogous manner to Example 18 by substituting 2-amino-3-(4-chlorophenyl)-1-propanol for pyrrolidin-3-yl-carbamic acid tert-butyl ester in step (h) and 2,3-dichlorophenylboronic acid for 3-chlorophenylboronic acid in step (i). MS (ES+) m/z 588.0 [M+H]⁺.

### Example 31

### Preparation of 2-(4-Amino-furazan-3-yl)-1-ethyl-4-(2-chloro-phenyl)-1H-imidazol-4,5-c]pyridine-7-carboxylic acid [1-(4-chloro-benzyl)-2-hydroxy-ethyl]-amide trifluoroacetate

The title compound was prepared in an analogous manner to Example 18 by substituting 2-amino-3-(4-chlorophenyl)-1-propanol for pyrrolidin-3-yl-carbamic acid tert-butyl ester in step (h) and 2-chlorophenylboronic acid for 3-chlorophenylboronic acid in step (i). MS (ES+) m/z 552.0 [M+H]⁺.

### Example 32

### Preparation of 2-(4-Amino-furazan-3-yl)-1-ethyl-4-(2-hydroxy-phenyl)-1H-imidazo[4,5-c]pyridine-7-carboxylic acid [1-(4-chloro-benzyl)-2-hydroxy-ethyl]-amide trifluoroacetate

The title compound was prepared in an analogous manner to Example 18 by substituting 2-amino-3-(4-chlorophenyl)-1-propanol for pyrrolidin-3-yl-carbamic acid tert-butyl ester in step (h) and 2-hydroxyphenylboronic acid for 3-chlorophenylboronic acid in step (i). MS (ES+) m/z 534.0 [M+H]⁺.

### Example 33

### Preparation of 2-(4-Amino-furazan-3-yl)-4-(3-chloro-phenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h). MS (ES+) m/z 453.0 [M+H]⁺.

### Example 34

### Preparation of 2-(4-Amino-furazan-3-yl)-4-phenyl-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and phenylboronic acid for 3-chlorophenylboronic acid in step (i). MS (ES+) m/z 419.0 [M+H]⁺.

### Example 35

### Preparation of 2-(4-Amino-furazan-3-yl)-4-(5-chloro-thiophen-2-yl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 5-chloro-2-thienylboronic acid for 3-chlorophenylboronic acid in step (i). MS (ES+) m/z 459.0 [M+H]⁺.

### Example 36

### Preparation of 2-(4-Amino-furazan-3-yl)-4-(2-amino-phenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 2-aminophenylboronic acid for 3-chlorophenylboronic acid in step (i). MS (ES+) m/z 434.0 [M+H]⁺.

### Example 37

### Preparation of 2-(4-Amino-furazan-3-yl)-4-(3-amino-phenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 3-aminophenylboronic acid for 3-chlorophenylboronic acid in step (i). MS (ES+) m/z 434.0 [M+H]⁺.

### Example 38

### Preparation of 2-(4-Amino-furazan-3-yl)-4-(3-bromo-phenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 3-bromophenylboronic acid for 3-chlorophenylboronic acid in step (i). MS (ES+) m/z 497.0 [M+H]⁺.

### Example 39

### Preparation of 2-(4-Amino-furazan-3-yl)-4-(1-naphthalenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 1-napthylboronic acid for 3-chlorophenylboronic acid in step (i). MS (ES+) m/z 469.0 [M+H]⁺.

### Example 40

### Preparation of 2-(4-Amino-furazan-3-yl)-4-(thiophen-2-yl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 2-thienylboronic acid for 3-chlorophenylboronic acid in step (i). MS (ES+) m/z 425.0 [M+H]⁺.

### Example 41

### Preparation of 2-(4-Amino-furazan-3-yl)-4-(3,4-methylenedioxyphenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 1,3-benzodioxol-5-ylboronic acid for 3-chlorophenylboronic acid in step (i). MS (ES+) m/z 463.0 [M+H]⁺.

### Example 42

### Preparation of 2-(4-Amino-furazan-3-yl)-4-(3,5-dichloro-phenyl)-1-ethyl-1H-imidazo[4,5-c]pyridine-7-carboxylic acid pyrrolidin-3-ylamide trifluoroacetate

The title compound was prepared in an analogous manner to Example 18 by substituting 1,1-dimethylethyl 3-amino-1-pyrrolidinecarboxylate for pyrrolidin-3-yl-carbamic acid tert-butyl ester in step (h) and 3,5-dichlorophenylboronic acid for 3-chlorophenylboronic acid in step (i). MS (ES+) m/z 487.0 [M+H]⁺.

### Example 43

### Preparation of 4-[7-[(3-amino-1-pyrrolidinyl)carbonyl]-4-(4-biphenylyl)-1-ethyl-1H-imidazo[4,5-c]pyridin-2-yl]-1,2,5-oxadiazol-3-amine trifluoroacetate

The title compound was prepared in an analogous manner to Example 18 by substituting 4-biphenylboronic acid for 3-chlorophenylboronic acid in step (i).

MS (ES+) m/z 495.0 [M+H]⁺.

### Example 44

### Preparation of 4-[7-[(3-amino-1-pyrrolidinyl)carbonyl]-4-(3-chlorophenyl)-1-(cyclopropylmethyl)-1H-imidazo[4,5-c]pyridin-2-yl-1,2,5-oxadiazol-3-amine trifluoroacetate

The title compound was prepared in an analogous manner to Example 18 by substituting 3-bromo-N-(cyclopropylmethyl)-5-nitro-4-pyridinamine for ethyl (3-bromo-5-nitropyridin-4-yl)amine in step (a). MS (ES+) m/z 479.2 [M+H]⁺.

### Example 45

### Preparation of 4-[7-[(3-amino-1-pyrrolidinyl)carbonyl]-4-(2,4-dichlorophenyl)-1-ethyl-1H-imidazo[4,5-c]pyridin-2-yl]-1,2,5-oxadiazol-3-amine trifluoroacetate

The title compound was prepared in an analogous manner to Example 18 by substituting 2,4-dichlorophenylboronic acid for 3-chlorophenylboronic acid in step (i). MS (ES+) m/z 407.0 [M+H]⁺.

### Example 46

### Preparation of 2-(2-(4-amino-1,2,5-oxadiazol-3-yl)-7-[(3-amino-1-pyrrolidinyl)carbonyl-1-ethyl-1H-imidazo[4,5-c]pyridin-4-yl}phenol trifluoroacetate

The title compound was prepared in an analogous manner to Example 18 by substituting 2-hydroxyphenylboronic acid for 3-chlorophenylboronic acid in step (i). MS (ES+) m/z 435.0 [M+H]⁺.

### Example 47

### Preparation of 4-[7-[(3-amino-1-pyrrolidinyl)carbonyl]-4-(2-chlorophenyl)-1-ethyl-1H-imidazo[4,5-c]pyridin-2-yl]-1,2,5-oxadiazol-3-amine trifluoroacetate

The title compound was prepared in an analogous manner to Example 18 by substituting 2-chlorophenylboronic acid for 3-chlorophenylboronic acid in step (i).

MS (ES+) m/z 453.0 [M+H]⁺.

### Example 48

### Preparation of (2-[2-(4-amino-1,2,5-oxadiazol-3-yl)-7-[(3-amino-1-pyrrolidinyl)carbonyl]-1-ethyl-1H-imidazo[4,5-c]pyridin-4-yl]phenyl)methanol trifluoroacetate

The title compound was prepared in an analogous manner to Example 18 by substituting 2-(hydroxymethyl)phenylboronic acid for 3-chlorophenylboronic acid in step (i). MS (ES+) m/z 449.0 [M+H]⁺.

### Example 49

### Preparation of 4-(1-ethyl-7-{3-(methylamino)-1-pyrrolidinyl]carbonyl}-4-phenyl-1H-imidazo[4,5-c]pyridin-2-yl)-1,2,5-oxadiazol-3-amine trifluoroacetate

The title compound was prepared in an analogous manner to Example 18 by substituting 1,1-dimethylethyl methyl(3-pyrrolidinyl)carbamate for pyrrolidin-3-yl-carbamic acid tert-butyl ester in step (h) and phenylboronic acid for 3-chlorophenylboronic acid in step (i). MS (ES+) m/z 433.0 [M+H]⁺.

### Example 50

### Preparation of 4-[7-[(3-amino-1-pyrrolidinyl)carbonyl]-1-ethyl-4-(4-methylphenyl)-1H-imidazo[4,5-c]pyridin-2-yl-1,2,5-oxadiazol-3-amine trifluoroacetate

The title compound was prepared in an analogous manner to Example 18 by substituting 4-methylphenylboronic acid for 3-chlorophenylboronic acid in step (i). MS (ES+) m/z 433.0 [M+H]⁺.

### Example 51

### Preparation of 4-[7-[(3-amino-1-pyrrolidinyl)carbonyl]-4-(2,5-dichlorophenyl)-1-ethyl-1H-imidazo[4,5-c]pyridin-2-yl]-1,2,5-oxadiazol-3-amine trifluoroacetate

The title compound was prepared in an analogous manner to Example 18 by substituting 2,5-dichlorophenylboronic acid for 3-chlorophenylboronic acid in step (i). MS (ES+) m/z 487.0 [M+H]⁺.

### Example 52

### Preparation of 4-[7-[(3-amino-1-pyrrolidinyl)carbonyl-4-(1-benzothien-2-yl)-1-ethyl-1H-imidazo[4,5-c]pyridin-2-yl]-1,2,5-oxadiazol-3-amine trifluoroacetate

The title compound was prepared in an analogous manner to Example 18 by substituting 1-benzothien-2-ylboronic acid for 3-chlorophenylboronic acid in step (i). MS (ES+) m/z 475.0 [M+H]⁺.

### Example 53

### Preparation of 4-17-[(3-amino-1-pyrrolidinyl)carbonyl]-1-ethyl-4-[4-(methyloxy)phenyl]-1H-imidazo[4,5-c]pyridin-2-yl}-1,2,5-oxadiazol-3-amine trifluoroacetate

The title compound was prepared in an analogous manner to Example 18 by substituting 4-methoxyphenylboronic acid for 3-chlorophenylboronic acid in step (i). MS (ES+) m/z 449.0 [M+H]⁺.

### Example 54

### Preparation of 4-[7-[(3-amino-1-pyrrolidinyl)carbonyl]-4-(4-chlorophenyl)-1-ethyl-1H-imidazo[4,5-c]pyridin-2-yl-1,2,5-oxadiazol-3-amine trifluoroacetate

The title compound was prepared in an analogous manner to Example 18 by substituting 4-chlorophenylboronic acid for 3-chlorophenylboronic acid in step (i).

MS (ES+) m/z 453.0 [M+H]⁺.

### Example 55

### Preparation of 2-(4-amino-1,2,5-oxadiazol-3-yl)-4-(3-chlorophenyl)-1-(cyclopropylmethyl)-N-[2-[(phenylmethyl)amino]ethyl]-1H-imidazo[4,5-c]pyridine-7-carboxamide trifluoroacetate

The title compound was prepared in an analogous manner to Example 18 by substituting 3-bromo-N-(cyclopropylmethyl)-5-nitro-4-pyridinamine for ethyl (3-bromo-5-nitropyridin-4-yl)amine in step (a) and 1,1-dimethylethyl (2-aminoethyl)(phenylmethyl)carbamate for pyrrolidin-3-yl-carbamic acid tert-butyl ester in step (h). MS (ES+) m/z 543.4 [M+H]⁺.

### Example 56

### Preparation of 3-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol trifluoroacetate

The title compound was prepared in an analogous manner to Example 18 by substituting 3-hydroxyphenylboronic acid for 3-chlorophenylboronic acid in step (i). MS (ES+) m/z 435.0 [M+H]⁺.

### Example 57

### Preparation of 4-[2-(4-amino-1,2,5-oxadiazol-3-yl)-7-[(3-amino-1-pyrrolidinyl)carbonyl]-1-ethyl-1H-imidazo[4,5-c]pyridin-4-yl]benzonitrile trifluoroacetate

The title compound was prepared in an analogous manner to Example 18 by substituting 4-cyanophenylboronic acid for 3-chlorophenylboronic acid in step (i).

MS (ES+) m/z 444.0 [M+H]⁺.

### Example 58

### Preparation of 1-[2-(4-Amino-furazan-3-yl)-4-phenyl-1-piperidin-4-yl-1-H-imidazo[4,5-c]pyridin-7-yl]-1-(3-amino-pyrrolidin-1-yl)-methanone

The title compound was prepared in an analogous manner to Example 18 by substituting 1,1-dimethylethyl 4-[(3-bromo-5-nitro-4-pyridinyl)amino]-1-piperidinecarboxylate for ethyl (3-bromo-5-nitropyridin-4-yl)amine in step (a) and phenylboronic acid for 3-chlorophenylboronic acid in step (i). MS (ES+) m/z 474.0 [M+H]⁺.

### Example 59

### Preparation of 4-(4-(3-chlorophenyl)-1-ethyl-7-{[3-(methylamino)-1-pyrrolidinyl]carbonyl}-1H-imidazo[4,5-c]pyridin-2-yl)-1,2,5-oxadiazol-3-amine trifluoroacetate

The title compound was prepared in an analogous manner to Example 18 by substituting 1,1-dimethylethyl methyl(3-pyrrolidinyl)carbamate for pyrrolidin-3-yl-carbamic acid tert-butyl ester in step (h). MS (ES+) m/z 467.0 [M+H]⁺.

### Example 60

### Preparation of 4-(4-(2,5-dichlorophenyl)-1-ethyl-7-{[3-(methylamino)-1-pyrrolidinyl]carbonyl}-1H-imidazo[4,5-c]pyridin-2-yl)-1,2,5-oxadiazol-3-amine trifluoroacetate

The title compound was prepared in an analogous manner to Example 18 by substituting 1,1-dimethylethyl methyl(3-pyrrolidinyl)carbamate for pyrrolidin-3-yl-carbamic acid tert-butyl ester in step (h) and 2,5-dichlorophenylboronic acid for 3-chlorophenylboronic acid in step (i). MS (ES+) m/z 501.0 [M+H]⁺.

### Example 61

### Preparation of 2-(4-amino-1,2,5-oxadiazol-3-yl)-4-(3-chlorophenyl)-1-(cylopropylmethyl-N-[3-(dimethylaminopropyl)-1H-imidazo[4,5-c]pyridine-7-carboxamide trifluoroacetate

The title compound was prepared in an analogous manner to Example 18 by substituting 3-bromo-N-(cyclopropylmethyl)-5-nitro-4-pyridinamine for ethyl (3-bromo-5-nitropyridin-4-yl)amine in step (a) and N,N-dimethyl-1,3-propanediamine for pyrrolidin-3-yl-carbamic acid tert-butyl ester in step (h). MS (ES+) m/z 496.4 [M+H]⁺.

### Example 62

### Preparation of 4-[7-[(3-amino-1-pyrrolidinyl)carbonyl]-1-ethyl-4-(1H-pyrrol-2-yl)-1H-imidazo[4,5-c]pyridin-2-yl]-1,2,5-oxadiazol-3-amine trifluoroacetate

The title compound was prepared in an analogous manner to Example 18 by substituting 1H-pyrrol-2-ylboronic acid for 3-chlorophenylboronic acid in step (i).

MS (ES+) m/z 408.0 [M+H]⁺.

### Example 63

### Preparation of 4-[7-[(3-amino-1-pyrrolidinyl)carbonyl]-4-(4-bromophenyl)-1-ethyl-1H-imidazo[4,5-c]pyridin-2-yl]-1,2,5-oxadiazol-3-amine trifluoroacetate

The title compound was prepared in an analogous manner to Example 18 by substituting 4-bromophenylboronic acid for 3-chlorophenylboronic acid in step (i). MS (ES+) m/z 497.0 [M+H]⁺.

### Example 64

### Preparation of 4-[7-[(3-amino-1-pyrrolidinyl)carbonyl]-4-phenyl-1-(4-piperidinyl)-1H-imidazo[4,5-c]pyridin-2-yl]-1,2,5-oxadiazol-3-amine

The title compound was prepared in an analogous manner to Example 18 by substituting 1,1-dimethylethyl 4-[(3-bromo-5-nitro-4-pyridinyl)amino]-1-piperidinecarboxylate for ethyl (3-bromo-5-nitropyridin-4-yl)amine in step (a) and phenylboronic acid for 3-chlorophenylboronic acid in step (i). MS (ES+) m/z 474.0 [M+H]⁺.

### Example 65

### Preparation of 4-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol trifluoroacetate

To a stirred solution of the compound of Example 53 (140 mg, 0.21 mmol) in methylene chloride (10 mL) at −78° C. was added dropwise boron tribromide (2.1 mL of 1 M solution in methylene chloride, 2.1 mmol). The reaction mixture was evaporated three times from methanol. Purification by preparative reverse phase HPLC (acetonitrile/water gradient with 0.1% TFA) afforded the title compound (51 mg, 56%). MS: (M+H)⁺=m/z 435.

### Example 66

### Preparation of 2-[2-(4-amino-1,2,5-oxadiazol-3-yl)-7-[(3-amino-1-pyrrolidinyl)carbonyl]-1-ethyl-1H-imidazo[4,5-c]pyridin-4-yl]-4-chlorophenol trifluoroacetate

The title compound was prepared in an analogous fashion to the preparation of the compound of Example 65 by substituting the compound of Example 53 with 4-{7-[(3-amino-1-pyrrolidinyl)carbonyl]-4-[5-chloro-2-(methyloxy)phenyl]-1-ethyl-1H-imidazo[4,5-c]pyridin-2-yl}-1,2,5-oxadiazol-3-amine.

MS: (M+H)⁺=m/z 469.

### Example 67

### Preparation of 4-[7-[(3-amino-1-pyrrolidinyl)carbonyl]-1-(1-methylethyl)-4-phenyl-1H-imidazo[4,5-c]pyridin-2-yl]-1,2,5-oxadiazol-3-amine trifluoroacetate

### a) 6-Chloro-4-isopropylamino-5-nitro-nicotinic acid ethyl ester

To a solution of 4,6-dichloro-5-nitro-nicotinic acid ethyl ester (1.305 g, 4.92 mmol) in dichloromethane (30 mL) at 0° C. was added isopropylamine (0.755 mL, 5.41 mmol). The mixture was then stirred at ambient temperature for 0.5 h, at which time it was concentrated in vacuo to provide the product as an orange solid (1.397 g, 99% yield). MS (ES+) m/z 288.2 (M+H)⁺.

### b) 4-Isopropylamino-5-nitro-6-phenyl-nicotinic acid ethyl ester

A solution of the compound of Example 67(a) (708 mg, 2.46 mmol), phenylboronic acid (600 mg, 4.92 mmol), and dichlorobis(triphenylphosphine)palladium(II) (173 mg, 0.246 mmol) in dioxane (23 mL) was treated with sodium carbonate (2 M aqueous solution, 3.94 mL, 7.88 mmol). The resultant biphasic mixture was vigorously stirred in a sealed tube at 100° C. for 3.5 h. The mixture was cooled to ambient temperature, concentrated, and purified on silica gel (50:1-30:1 dichloromethane/methanol) to give the desired product as a light brown oil (739 mg, 91% yield). MS (ES+) m/z 330.2 (M+H)⁺.

### c) 5-Amino-4-isopropylamino-6-phenyl-nicotinic acid ethyl ester

A mixture of the compound of Example 67(b) (735 mg, 2.23 mmol) and palladium on carbon (10 wt. %, 20 mg) in absolute ethanol (20 mL) was stirred at ambient temperature under hydrogen gas (1 atm) for 16 h, at which time the flask was flushed with argon. The catalyst was filtered off on a pad of celite, and the filtrate was concentrated in vacuo to afford the product as a dark yellow oil (639 mg, 96% yield). MS (ES+) m/z 300.2 (M+H)⁺.

### d) 5-(2-Cyano-ethanoylamino)-4-isopropylamino-6-phenyl-nicotinic acid ethyl ester

A solution of the compound of the Example 67(c) (635 mg, 2.12 mmol), cyanoacetic acid (451 mg, 5.30 mmol), 4-methylmorpholine (1.17 mL, 1.06 mmol) in dimethylformamide (10 mL) was treated with 1-(3-dimethylaminopropyl)-3-ethylcarbodiimide hydrochloride (1.016 g, 5.30 mmol), and the resultant mixture was stirred under argon at 45° C. for 3 hours. The reaction was then diluted with water (30 mL) and extracted with ethyl acetate (3×50 mL). The combined organic extracts were sequentially washed with saturated aqueous sodium bicarbonate solution (1×25 mL), water (1×25 mL), brine (1×50 mL), and then dried over magnesium sulfate and concentrated in vacuo to furnish the product (723 mg, 93% yield) as a pale brown oil. MS (ES+) m/z 367.4 (M+H)⁺.

### e) 2-Cyanomethyl-1-isopropyl-4-phenyl-1H-imidazo[4,5-c]pyridine-7-carboxylic acid ethyl ester

A mixture of the compound of the Example 67(d) (723 mg, 1.97 mmol) and acetic acid (15 mL) was stirred in a sealed tube at 100° C. for 1 h. Concentration in vacuo, followed by silica gel chromatography provided the product (500 mg, 73% yield) as an ivory solid. MS (ES+) m/z 349.2 (M+H)⁺.

### f) 2-(1-Cyano-1-hydroxyimino-methyl)-1-isopropyl-4-phenyl-1H-imidazo[4,5-c]pyridine-7-carboxylic acid ethyl ester

To a solution of the compound of the Example 67(e) (530 mg, 1.52 mmol) in acetic acid (11 mL) and water (1.5 mL) was added sodium nitrite (210 mg, 3.04 mmol), portionwise over 2 min. The reaction was stirred at ambient temperature for 16 h, at which time it was concentrated in vacuo. The residue was dissolved in dichloromethane (100 mL) and washed with saturated aqueous sodium bicarbonate solution (1×20 mL) and water (1×20 mL). Drying over anhydrous magnesium sulfate, followed by concentration in vacuo, gave the product (574 mg, 100% yield) as a pale yellow solid. MS (ES+) m/z 378.4 (M+H)⁺.

### g) Ethyl 2-(4-amino-1,2,5-oxadiazol-3-yl)-1-(1-methylethyl)-4-phenyl-1H-imidazo[4,5-c]pyridine-7-carboxylate

A mixture of the compound of the Example 67(f) (574 mg, 1.52 mmol), triethylamine (2 mL, 14.3 mmol), and hydroxylamine (50 wt. % solution in water, 0.120 mL, 1.96 mmol) in dioxane (30 mL) was heated in a sealed tube at 110° C. for 6 h. The mixture was cooled to ambient temperature, concentrated in vacuo, and purified on silica gel (50:1→30:1 dichloromethane/methanol) to afford the product (445 mg, 74% yield) a as pale yellow solid. MS (ES+) m/z 393.4 (M+H)⁺.

### h) 2-(4-Amino-1,2,5-oxadiazol-3-yl)-1-(1-methylethyl)-4-phenyl-1H-imidazo[4,5-c]pyridine-7-carboxylic acid

To a solution of the compound of the Example 67(g) (440 mg, 1.12 mmol) in 4:1 methanol/tetrahydrofuran was added 6 N aqueous sodium hydroxide solution (2.75 mL, 16.5 mmol). The mixture was vigorously stirred at ambient temperature for 3 hours, at which time it was concentrated in vacuo and diluted with water (20 mL). The pH was adjusted to 7 by addition of 6 N hydrochloric acid (2.75 mL), and the aqueous phase was extracted with ethyl acetate (3×25 mL). The combined organic extracts were washed with brine (1×15 mL), dried over magnesium sulfate and concentrated in vacuo to furnish the product (380 mg, 93% yield) as a pale yellow solid. MS (ES+) m/z 365.2 (M+H)⁺.

### i) 1,1-Dimethylethyl (1-{[2-(4-amino-1,2,5-oxadiazol-3-yl)-1-(1-methylethyl)-4-phenyl-1H-imidazo[4,5-c]pyridin-7-yl]carbonyl}-3-pyrrolidinyl)carbamate

To a solution of the compound of the Example 67(h) (64 mg, 0.176 mmol), pyrrolidin-3-yl-carbamic acid tert-butyl ester (66 mg, 0.352 mmol), 4-methylmorpholine (0.1 mL, 0.909 mmol), 1-hydroxy-7-azabenzotriazole (48 mg, 0.352 mmol) in dimethylformamide (3 mL) was added 1-(3-dimethylaminopropyl)-3-ethylcarbodiimide hydrochloride (68 mg, 0.352 mmol). The resultant mixture was stirred under argon at 45° C. for 2.5 h, at which time it was diluted with ethyl acetate (30 mL) and washed with water (3×10 mL). The organic phase was washed with brine (1×10 mL), dried over magnesium sulfate, and concentrated. Purification on silica gel (20:1→10:1 dichloromethane/methanol) provided the product (85 mg, 91% yield) as a pale yellow oil that solidified upon standing. MS (ES+) m/z 533.6 (M+H)⁺.

### j) 4-[7-[(3-Amino-1-pyrrolidinyl)carbonyl]-1-(1-methylethyl)-4-phenyl-1H-imidazo[4,5-c]pyridin-2-yl]-1,2,5-oxadiazol-3-amine

A solution of the compound of the Example 67(i) (85 mg, 0.160 mmol) in dichloromethane 3 mL was treated with trifluoroacetic acid (0.6 mL, 7.79 mmol). The reaction was stirred at ambient temperature for 1.5 h, at which time it was diluted with toluene (5 mL) and concentrated in vacuo to give the product (96 mg, 91% yield) as a pale tan solid. MS (ES+) m/z 433.6 (M+H)⁺.

### Example 68

### Preparation of 2-(4-amino-1,2,5-oxadiazol-3-yl)-1-ethyl-N-methyl-N-(1-methyl-4-Piperidinyl)-4-phenyl-1H-imidazo[4,5-c]pyridine-7-carboxamide trifluoroactetate

The title compound was prepared in an analogous manner to Example 67 by substituting ethylamine for isopropylamine in step (a) and N,1-dimethyl-3-pyrrolidinamine for pyrrolidin-3-yl-carbamic acid tert-butyl ester in step (i). MS (ES+) m/z 461.0 [M+H]⁺.

### Example 69

### Preparation of 4-{1-(4-aminobutyl)-7-[(3-amino-1-pyrrolidinyl)carbonyl]-4-phenyl-1H-imidazo[4,5-c]pyridin-2-yl}-1,2,5-oxadiazol-3-amine trifluoroactetate

The title compound was prepared in an analogous manner to Example 67 by substituting 1,1-dimethylethyl (4-aminobutyl)carbamate for isopropylamine in step (a). MS (ES+) m/z 462.0 [M+H]⁺.

### Example 70

### Preparation of 4-(1-(4-aminobutyl)-7-{[3-(methylamino)-1-pyrrolidinyl]carbonyl}-4-phenyl-1H-imidazo[4,5-c]pyridin-2-yl)-1,2,5-oxadiazol-3-amine trifluoroactetate

The title compound was prepared in an analogous manner to Example 67 by substituting 1,1-dimethylethyl (4-aminobutyl)carbamate for isopropylamine in step (a) and 1,1-dimethylethyl methyl(3-pyrrolidinyl)carbamate for pyrrolidin-3-yl-carbamic acid tert-butyl ester in step (i). MS (ES+) m/z 476.0 [M+H]⁺.

### Example 71

### Preparation of 1-(4-aminobutyl)-2-(4-amino-1,2,5-oxadiazol-3-yl)-4-phenyl-N-{2-[(phenylmethyl)amino]ethyl}-1H-imidazo[4,5-c]pyridine-7-carboxamide trifluoroactetate

The title compound was prepared in an analogous manner to Example 67 by substituting 1,1-dimethylethyl (4-aminobutyl)carbamate for isopropylamine in step (a) and 1,1-dimethylethyl (2-aminoethyl)(phenylmethyl)carbamate for pyrrolidin-3-yl-carbamic acid tert-butyl ester in step (i). MS (ES+) m/z 526.0 [M+H]⁺.

### Example 72

### Preparation of 2-(4-amino-1,2,5-oxadiazol-3-yl)-1-(1-methylethyl)-4-phenyl-N-3-pyrrolidinyl-1H-imidazo[4,5-c]pyridine-7-carboxamide trifluoroactetate

The title compound was prepared in an analogous manner to Example 67 by substituting 1,1-dimethylethyl 3-amino-1-pyrrolidinecarboxylate for pyrrolidin-3-yl-carbamic acid tert-butyl ester in step (i). MS (ES+) m/z 433.4 [M+H]⁺.

### Example 73

### Preparation of 4-[7-[[3-(methylamino)-1-pyrrolidinyl]carbonyl]-1-(1-methylethyl)-4-phenyl-1H-imidazo[4,5-c]pyridin-2-yl]-1,2,5-oxadiazol-3-amine trifluoroactetate

The title compound was prepared in an analogous manner to Example 67 by substituting 1,1-dimethylethyl methyl(3-pyrrolidinyl)carbamate for pyrrolidin-3-yl-carbamic acid tert-butyl ester in step (i). MS (ES+) m/z 447.6 [M+H]⁺.

### Example 74

### Preparation of 2-(4-amino-1,2,5-oxadiazol-3-yl)-N-(3-aminopropyl)-1-(1-methylethyl)-4-phenyl-1H-imidazo[4,5-c]pyridine-7-carboxamide trifluoroactetate

The title compound was prepared in an analogous manner to Example 67 by substituting 1,1-dimethylethyl (4-aminobutyl)carbamate for isopropylamine in step (a) and ethanolamine for pyrrolidin-3-yl-carbamic acid tert-butyl ester in step (i). MS (ES+) m/z 421.2 [M+H]⁺.

### Example 75

### Preparation of 2-(4-amino-1,2,5-oxadiazol-3-yl)-1-(1-methylethyl)-4-phenyl-N-2-propen-1-yl-1H-imidazo[4,5-c]pyridine-7-carboxamide

The title compound was prepared in an analogous manner to Example 67 by substituting allyl amine for pyrrolidin-3-yl-carbamic acid tert-butyl ester in step (i) and omitting step (j). MS (ES+) m/z 404.4 [M+H]⁺.

### Example 76

### Preparation of 2-(4-amino-1,2,5-oxadiazol-3-yl)-1-ethyl-N-[3-(4-morpholinyl)propyl]-4-phenyl-1H-imidazo[4,5-c]pyridine-7-carboxamide hydrochloride

The title compound was prepared in an analogous manner to Example 67 by substituting ethylamine for isopropylamine in step (a), 3-(4-morpholinyl)-1-propanamine for pyrrolidin-3-yl-carbamic acid tert-butyl ester in step (i) and 4N HCl/dioxane for trifluoroacetic acid and CH₂Cl₂ in step (j). MS (ES+) m/z 477.0 [M+H]⁺.

### Example 77

### Preparation of 2-(4-amino-1,2,5-oxadiazol-3-yl)-1-ethyl-N-[2-(1H-imidazol-4-yl)ethyl]-4-phenyl-1H-imidazo[4,5-c]pyridine-7-carboxamide hydrochloride

The title compound was prepared in an analogous manner to Example 67 by substituting ethylamine for isopropylamine in step (a), 2-(1H-imidazol-4-yl)ethanamine for pyrrolidin-3-yl-carbamic acid tert-butyl ester in step (i) and 4N HCl/dioxane for trifluoroacetic acid and CH₂Cl₂ in step (j). MS (ES+) m/z 444.0 [M+H]⁺.

### Example 78

### Preparation of 2-(4-amino-1,2,5-oxadiazol-3-yl)-1-ethyl-N-[3-(4-methyl-1-piperazinyl)propyl]-4-Phenyl-1H-imidazo[4,5-c]Pyridine-7-carboxamide trifluoroacetate

The title compound was prepared in an analogous manner to Example 67 by substituting ethylamine for isopropylamine in step (a) and 3-(4-methyl-1-piperazinyl)-1-propanamine for pyrrolidin-3-yl-carbamic acid tert-butyl ester in step (i). MS (ES+) m/z 490.0 [M+H]⁺.

### Example 79

### Preparation of N-(1-{[2-(4-amino-1,2,5-oxadiazol-3-yl)-1-ethyl-4-phenyl-1H-imidazo[4,5-c]pyridin-7-yl]carbonyl}-3-pyrrolidinyl)-N-methylacetamide trifluoroacetate

The title compound was prepared in an analogous manner to Example 67 by substituting ethylamine for isopropylamine in step (a) and N-methyl-N-3-pyrrolidinylacetamide for pyrrolidin-3-yl-carbamic acid tert-butyl ester in step (i).

MS (ES+) m/z 475.0 [M+H]⁺.

### Example 80

### Preparation of 2-(4-amino-1,2,5-oxadiazol-3-yl)-N-[3-(dimethylamino)propyl]-1-ethyl-4-phenyl-1H-imidazo[4,5-c]pyridine-7-carboxamide hydrochloride

The title compound was prepared in an analogous manner to Example 67 by substituting ethylamine for isopropylamine in step (a), N,N-dimethyl-1,3-propanediamine for pyrrolidin-3-yl-carbamic acid tert-butyl ester in step (i) and 4N HCl/dioxane for trifluoroacetic acid and CH₂Cl₂ in step (j). MS (ES+) m/z 435.0 [M+H]⁺.

### Example 81

### Preparation of 2-(4-amino-1,2,5-oxadiazol-3-yl)-1-cyclopentyl-4-phenyl-N-3-pyrrolidinyl-1H-imidazo[4,5-c]pyridine-7-carboxamide trifluoroacetate

The title compound was prepared in an analogous manner to Example 67 by substituting cyclopentylamine for isopropylamine in step (a) and 1,1-dimethylethyl 3-amino-1-pyrrolidinecarboxylate for pyrrolidin-3-yl-carbamic acid tert-butyl ester in step (i). MS (ES+) m/z 459.2 [M+H]⁺.

### Example 82

### Preparation of 4-[7-[(3-amino-1-pyrrolidinyl)carbonyl]-1-cyclopentyl-4-phenyl-1H-imidazo[4,5-c]pyridin-2-yl]-1,2,5-oxadiazol-3-amine trifluoroacetate

The title compound was prepared in an analogous manner to Example 67 by substituting cyclopentylamine for isopropylamine in step (a). MS (ES+) m/z 459.4 [M+H]⁺.

### Example 83

### Preparation of 4-(1-cyclopentyl-7-{[3-(methylamino)-1-pyrrolidinyl]carbonyl-4-phenyl-1H-imidazo[4,5-c]pyridin-2-yl)-1,2,5-oxadiazol-3-amine trifluoroacetate

The title compound was prepared in an analogous manner to Example 67 by substituting cyclopentylamine for isopropylamine in step (a) and 1,1-dimethylethyl methyl(3-pyrrolidinyl)carbamate for pyrrolidin-3-yl-carbamic acid tert-butyl ester in step (i). MS (ES+) m/z 473.4 [M+H]⁺.

### Example 84

### Preparation of (3R)-1-{2-(4-amino-1,2,5-oxadiazol-3-yl)-1-ethyl-4-phenyl-1H-imidazo[4,5-c]pyridin-7-yl]carbonyl}-3-pyrrolidinol hydrochloride

The title compound was prepared in an analogous manner to Example 67 by substituting ethylamine for isopropylamine in step (a), (3R)-3-pyrrolidinol for pyrrolidin-3-yl-carbamic acid tert-butyl ester in step (i) and 4N HCl/dioxane for trifluoroacetic acid and CH₂Cl₂ in step (j). MS (ES+) m/z 420.0 [M+H]⁺.

### Example 85

### Preparation of Preparation of 2-(4-amino-1,2,5-oxadiazol-3-yl)-N-[3-(diethylamino)propyl]-1-ethyl-4-phenyl-1H-imidazo[4,5-c]pyridine-7-carboxamide trifluoroacetate

The title compound was prepared in an analogous manner to Example 67 by substituting ethylamine for isopropylamine in step (a) and N,N-diethyl-1,3-propanediamine for pyrrolidin-3-yl-carbamic acid tert-butyl ester in step (i).

MS (ES+) m/z 463.0 [M+H]⁺.

### Example 86

### Preparation of 2-(4-amino-1,2,5-oxadiazol-3-yl)-1-ethyl-N-[3-(2-methyl-1-piperidinyl)propyl]-4-phenyl-1H-imidazo[4,5-c]pyridine-7-carboxamide hydrochloride

The title compound was prepared in an analogous manner to Example 67 by substituting ethylamine for isopropylamine in step (a), 3-(2-methyl-1-piperidinyl)-1-propanamine for pyrrolidin-3-yl-carbamic acid tert-butyl ester in step (i) and 4N HCl/dioxane for trifluoroacetic acid and CH₂Cl₂ in step (j). MS (ES+) m/z 489.0 [M+H]⁺.

### Example 87

### Preparation of 4-[7-[(3-amino-1-pyrrolidinyl)carbonyl]-1-(4-fluorophenyl)-4-phenyl-1H-imidazo[4,5-c]pyridin-2-yl]-1,2,5-oxadiazol-3-amine trifluoroacetate

The title compound was prepared in an analogous manner to Example 67 by substituting 4-fluoroaniline for isopropylamine in step (a). MS (ES+) m/z 485.0 [M+H]⁺.

### Example 88

### Preparation of N-(2-aminoethyl)-2-(4-amino-1,2,5-oxadiazol-3-yl)-1-(4-fluorophenyl)-4-phenyl-1H-imidazo[4,5-c]pyridine-7-carboxamide trifluoroacetate

The title compound was prepared in an analogous manner to Example 67 by substituting 4-fluoroaniline for isopropylamine in step (a) and 1,1-dimethylethyl (2-aminoethyl)carbamate for pyrrolidin-3-yl-carbamic acid tert-butyl ester in step (i).

MS (ES+) m/z 459.0 [M+H]⁺.

### Example 89

### Preparation of 4-{1-(4-aminophenyl)-7-[(3-amino-1-pyrrolidinyl)carbonyl]-4-phenyl-1H-imidazo[4,5-c]pyridin-2-yl}-1,2,5-oxadiazol-3-amine trifluoroacetate

The title compound was prepared in an analogous manner to Example 67 by substituting 1,1-dimethylethyl (4-aminophenyl)carbamate for isopropylamine in step (a). MS (ES+) m/z 482.0 [M+H]⁺.

### Example 90

### Preparation of 4-[7-{[3-(dimethylamino)-1-pyrrolidinyl]carbonyl}-4-phenyl-1-(2,2,2-trifluoroethyl)-1H-imidazo[4,5-c]pyridin-2-yl]-1,2,5-oxadiazol-3-amine trifluoroacetate

The title compound was prepared in an analogous manner to Example 67 by substituting 2,2,2-trifluoroethylamine for isopropylamine in step (a) and N,N-dimethyl-3-pyrrolidinamine for pyrrolidin-3-yl-carbamic acid tert-butyl ester in step (i). MS (ES+) m/z 501.0 [M+H]⁺.

### Example 91

### Preparation of 2-(4-amino-1,2,5-oxadiazol-3-yl)-1-ethyl-N-[2-(1-methyl-2-pyrrolidinyl)ethyl]-4-phenyl-1H-imidazo[4,5-c]pyridine-7-carboxamide trifluoroacetate

The title compound was prepared in an analogous manner to Example 67 by substituting ethylamine for isopropylamine in step (a) and 2-(1-methyl-2-pyrrolidinyl)ethanamine for pyrrolidin-3-yl-carbamic acid tert-butyl ester in step (i).

MS (ES+) m/z 461.0 [M+H]⁺.

### Example 92

### Preparation of 1-(1-[2-(4-amino-1,2,5-oxadiazol-3-yl)-1-ethyl-4-phenyl-1H-imidazo[4,5-c]pyridin-7-yl]carbonyl}-4-piperidinyl)-1,3-dihydro-2H-benzimidazol-2-one trifluoroacetate

The title compound was prepared in an analogous manner to Example 67 by substituting ethylamine for isopropylamine in step (a) and 1-(4-piperidinyl)-1,3-dihydro-2H-benzimidazol-2-one for pyrrolidin-3-yl-carbamic acid tert-butyl ester in step (i). MS (ES+) m/z 550.0 [M+H]⁺.

### Example 93

### Preparation of 1-{[2-(4-amino-1,2,5-oxadiazol-3-yl)-1-ethyl-4-phenyl-1H-imidazo[4,5-c]pyridin-7-yl]carbonyl}-3-piperidinecarboxamide hydrochloride

The title compound was prepared in an analogous manner to Example 67 by substituting ethylamine for isopropylamine in step (a), 3-piperidinecarboxamide for pyrrolidin-3-yl-carbamic acid tert-butyl ester in step (i) and 4N HCl/dioxane for trifluoroacetic acid and CH₂Cl₂ in step @). MS (ES+) m/z 461.0 [M+H]⁺.

### Example 94

### Preparation of N-(3-amino-2-hydroxypropyl)-2-(4-amino-1,2,5-oxadiazol-3-yl)-1-ethyl-4-phenyl-1H-imidazo[4,5-c]pyridine-7-carboxamide trifluoroacetate

The title compound was prepared in an analogous manner to Example 67 by substituting ethylamine for isopropylamine in step (a) and 1,1-dimethylethyl 5-(aminomethyl)-2,2-dimethyl-1,3-oxazolidine-3-carboxylate for pyrrolidin-3-yl-carbamic acid tert-butyl ester in step (i). MS (ES+) m/z 423.0 [M+H]⁺.

### Example 95

### Preparation of N-(2-amino-3-hydroxypropyl)-2-(4-amino-1,2,5-oxadiazol-3-yl)-1-ethyl-4-phenyl-1H-imidazo[4,5-c]pyridine-7-carboxamide hydrochloride

The title compound was prepared in an analogous manner to Example 67 by substituting ethylamine for isopropylamine in step (a), 11-dimethylethyl 4-(aminomethyl)-2,2-dimethyl-1,3-oxazolidine-3-carboxylate for pyrrolidin-3-yl-carbamic acid tert-butyl ester in step (i) and 4N HCl/dioxane for trifluoroacetic acid and CH₂Cl₂ in step (O). MS (ES+) m/z 423.0 [M+H]⁺.

### Example 96

### Preparation of (4-{[2-(4-amino-1,2,5-oxadiazol-3-yl)-1-ethyl-4-phenyl-1H-imidazo[4,5-c]pyridin-7-yl]carbonyl}-2-piperazinyl)methanol hydrochloride

The title compound was prepared in an analogous manner to Example 67 by substituting ethylamine for isopropylamine in step (a), 2[(methyloxy)methyl]piperazine for pyrrolidin-3-yl-carbamic acid tert-butyl ester in step (i) and 3M BCl₃ in MeOH for trifluoroacetic acid and CH₂Cl₂ in step (j).

MS (ES+) m/z 449.0 [M+H]⁺.

### Example 97

### Preparation of 4-[1-ethyl-7-([3-[(methyloxy)methyl]-1-piperazinylcarbonyl)-4-phenyl-1H-imidazo[4,5-c]pyridin-2-yl]-1,2,5-oxadiazol-3-amine hydrochloride

The title compound was prepared in an analogous manner to Example 67 by substituting ethylamine for isopropylamine in step (a), 2-[(methyloxy)methyl]piperazine for pyrrolidin-3-yl-carbamic acid tert-butyl ester in step (i) and 4N HCl/dioxane for trifluoroacetic acid and CH₂Cl₂ in step (O). MS (ES+) m/z 463.0 [M+H]⁺.

### Example 98

### Preparation of 4-(1-methyl-7-{[3-(methylamino)-1-pyrrolidinyl]carbonyl}-4-phenyl-1H-imidazo[4,5-c]pyridin-2-yl)-1,2,5-oxadiazol-3-amine hydrochloride

### a) (z)-2-Nitro-1-phenylethenamine

Triethylamine (8.4 mL, 0.06 mol) was added to a solution of methoxylamine-HCl (5.2 g, 0.0625 mol) in dimethylformamide (100 mL) at 0° C. in a ice bath., Nitrostyrene (7.50 g, 0.05 mol) was added and stirred at 0° C. for 15 min then at RT for 5 min. Remove the precipate by filtration and wash the solid with a small amount of DMF. Place the combined filtrate into an addition funnel and add dropwise over 30 min to potassium t-butoxide (16.8 g, 0.15 mol) in DMF (150 mL) at 0° C. Remove the bath and stir at RT for 30 min. Quench reaction with sat NH4Cl (50 mL). Reaction volume reduced in ½ in vacuo and extracted with CH₂Cl₂. Wash with water, brine, dry Na₂SO₄, filter and concentrate in vacuo to give the desired material as a yellow solid (7.6 g, 93%). MS (ES)⁺ m/e 165 [M+H]⁺.

### b) Diethyl {[(2-nitro-1-phenylethyl)amino]methylidene}propanedioate

Diethyl malonate (17 mL, 0.09 mol) was added to the compound of Example 98(a) in triethylamine (30 mL) in a pressure bottle. The reaction was placed into an oil bath at 120° C. and held for 1 hr. Remove from heat and concentrate in vacuo. Dissolve the residue in hot CH₂Cl₂ (50 mL) and add 8% ethyl acetate/hexane (200 mL). Allow to cool to RT and then place in an ice bath for 1 h. Collect precipitate and wash with cold 8% ethyl actate/hexane. Dry under vacuum and to give the desired material as a yellow solid (7.7 g, 80%). MS (ES)⁺ m/e 335 [M+H]⁺.

### c) Ethyl 4-hydroxy-5-nitro-6-phenyl-3-pyridinecarboxylate

The compound of Example 98(b) in diphenyl ether (70 mL) was heated to 260° C. for 20 min. with stirring. After cooling to RT, dilute with hexane (70 mL) and collect the resulting precipitate. Rinse with hexane and dry the precipitate under vacuum to give the desired product as an off-white solid (7.60 g, 81%). MS (ES)⁺ m/e 289 [M+H]⁺.

### d) Ethyl 4-chloro-5-nitro-6-phenyl-3-pyridinecarboxylate

The compound of Example 98(c) and POCl₃ (7 mL) was heated in a pressure bottle for 1 h at 115° C. The volatiles were removed in vacuo after allowing the reaction to cool to RT. Dissolve the residue in CH₂Cl₂ and filter through a plug of silica gel, flushing with additional CH₂Cl₂ (800 mL). The solvent was removed in vacuo to give the desired product as an oil that solidified on standing (3.10 g, 96%). MS (ES)⁺ m/e 307 [M+H]⁺.

### e) Ethyl 4-(methylamino)-5-nitro-6-phenyl-3-pyridinecarboxylate

To the compound of Example 98(d) and Et₃N (0.75 mL, 3.60 mmol) in ethanol (30 mL) was added a solution of MeNH₂ in THF (1.80 mL, 2.0 M, 3.60 mmol). After stirring at RT for 16 h, the solvent was removed in vacuo. The residue was dissolved in EtOAc and passed through a plug of silica gel eluting with 5% EtOAc/hexane. The solvent was removed to give the desired product as a solid (0.88 g, 98%). MS (ES)⁺ m/e 302 [M+H]⁺.

### f) Ethyl 5-amino-4-(methylamino)-6-phenyl-3-pyridinecarboxylate

To a solution of the compound of Example 98(e) in EtOH (30 mL) was added 10% Pd/C (0.1 g). The reaction vessel was fitted with a H₂ filled balloon and heated to 45° C. for 18 h. The reaction was allowed to cool to RT and the H₂ was vented. Celite and additional CH₂Cl₂ were added to the mixture. The solid material was removed by filtration. The solids were washed with 5% MeOH/CH₂Cl₂. The solvent was removed from the combined filtrate to give the desired product as a yellow solid (0.81 g, 100%). MS (ES)⁺ m/e 272 [M+H]⁺.

### g) 2-(4-Amino-1,2,5-oxadiazol-3-yl)-1-methyl-4-phenyl-1H-imidazo[4,5-c]pyridine-7-carboxylic acid

This compound was prepared in a manner analogous to the preparation of the compound of Example 67(h), except substituting the compound of Example 98(f) for the compound of Example 67(g). MS (ES+) m/z 337 (M+H)⁺.

### h) 1,1-Dimethylethyl (1-{[2-(4-amino-1,2,5-oxadiazol-3-yl)-1-methyl-4-phenyl-1H-imidazo[4,5-c]pyridin-7-yl]carbonyl}-3-pyrrolidinyl)methylcarbamate

This compound was prepared in a manner analogous to the preparation of the compound of Example 67(i), except substituting methyl-pyrrolidin-3-yl-carbamic acid dimethyl-ethyl ester for pyrrolidin-3-yl-carbamic acid tert-butyl ester. MS (ES+) m/z 519 (M+H)⁺.

### i) 4-(1-Methyl-7-{[3-(methylamino)-1-pyrrolidinyl]carbonyl}-4-phenyl-1H-imidazo[4,5-c]pyridin-2-yl)-1,2,5-oxadiazol-3-amine hydrochloride

The title compound was prepared in a manner analogous to the preparation of the compound of Example 670), except substituting the compound of Example 98(h) for the compound of Example 67(i) and substituting 4N HCl/dioxane for trifluoroacetic acid in CH₂Cl₂. MS (ES+) m/z 419 (M+H)⁺.

### Example 99

### Preparation of 4-{7-[(3-amino-1-pyrrolidinyl)carbonyl]-1-methyl-4-phenyl-1H-imidazo[4,5-c]pyridin-2-yl}-1,2,5-oxadiazol-3-amine hydrochloride

The title compound was prepared in an analogous manner to Example 98 by substituting 1,1-dimethylethyl 3-pyrrolidinylcarbamate for 1,1-dimethylethyl methyl(3-pyrrolidinyl)carbamate in step (h). MS (ES+) m/z 405.0 [M+H]⁺.

### Example 100

### Preparation of 4-(1-butyl-7-{3-(methylamino)-1-pyrrolidinyl)carbonyl}-4-phenyl-1H-imidazo[4,5-c]pyridin-2-yl)-1,2,5-oxadiazol-3-amine hydrochloride

The title compound was prepared in an analogous manner to Example 98 by substituting butylamine for methylamine in step (e). MS (ES+) m/z 461.0 [M+H]⁺.

### Example 101

### Preparation of 4-[7-[(3-amino-1-pyrrolidinyl)carbonyl]-1-butyl-4-phenyl-1H-imidazo[4,5-c]pyridin-2-yl]-1,2,5-oxadiazol-3-amine hydrochloride

The title compound was prepared in an analogous manner to Example 98 by substituting butylamine for methylamine in step (e) and 1,1-dimethylethyl 3-pyrrolidinylcarbamate for 1,1-dimethylethyl methyl(3-pyrrolidinyl)carbamate in step (h). MS (ES+) m/z 447.0 [M+H]⁺.

### Example 102

### Preparation of N-[2-(4-amino-1,2,5-oxadiazol-3-yl)-1-ethyl-4-phenyl-1H-imidazo[4,5-c]pyridin-7-yl]-4-piperidinecarboxamide trifluoroacetate

### a) Ethyl 2-(4-amino-1,2,5-oxadiazol-3-yl)-1-ethyl-4-phenyl-1H-imidazo[4,5-c]pyridine-7-carboxylate

This compound was prepared in a manner analogous to the preparation of the compound of Example 67(a) through 67(g), except substituting ethylamine for isopropylamine. MS (ES+) m/z 379 (M+H)⁺.

### b) Ethyl 2-[4-({[(1,1-dimethylethyl)oxy]carbonyl}amino)-1,2,5-oxadiazol-3-yl]-1-ethyl-4-phenyl-1H-imidazo[4,5-c]pyridine-7-carboxylate

A solution consisting of the compound of Example 102(a) (24.7 mmol) in 1,2-dichloroethane (140 mL) and pyridine (70 mL) with di-t-butyl dicarbonate (21.54 g, 98.8 mmol) and DMAP (3.01 g, 24.7 mmol) was stirred at 85° C. in a sealed flask for 1 h. The product mixture was partitioned between EtOAc and 1N HCl, the layers separated and the organic extract washed with 1N HCl then brine, dried (Na₂SO₄) and all volatiles removed in vacuo. The residue was triturated with EtOAc to afford the desired compound as beige solid. MS (ES+) m/z 479 (M+H)⁺.

### c) 2-[4-({[(1,1-Dimethylethyl)oxy]carbonyl}amino)-1,2,5-oxadiazol-3-yl]-ethyl-4-phenyl-1H-imidazo[4,5-c]pyridine-7-carboxylic acid

This compound was prepared in a manner analogous to the preparation of the compound of Example 67(h), except substituting the compound of Example 102(b) for the compound of Example 67(g). MS (ES+) m/z 451 (M+H)⁺.

### d) 1,1-Dimethylethyl [4-(7-amino-1-ethyl-4-phenyl-1H-imidazo[4,5-c]pyridin-2-yl)-1,2,5-oxadiazol-3-yl]carbamate

To a suspension of the compound of 102(c) (0.14 g, 0.30 mmol) in toluene (5 mL) at RT was added Et₃N (63 uL, 0.45 mmol) followed by diphenylphosphoryl azide(65 uL, 0.30 mmol). The mixture was stirred at RT for 15 min and then at reflux for 1 h. Water (0.5 mL) was added and the solution was heated to reflux for 24 h. After allowing the reaction to cool to RT, the solvent was in vacuo. The residue was diluted with CH₂Cl₂ (10 mL), washed with H₂O (2×5 mL) and brine (5 mL). Flash chromatography (2-5% CH₃OH/CH₂Cl₂, silica gel) gave 0.07 g (55%) of the desired compound. MS (ES+) m/z 422 (M+H)⁺.

### e) Phenylmethyl 4-[({2-[4-({[(1,1-dimethylethyl)oxy]carbonyl}amino)-1,2,5-oxadiazol-3-yl]-1-ethyl-4-phenyl-1H-imidazo[4,5-c]pyridin-7-yl}amino)carbonyl]-1-piperidinecarboxylate

A solution of the compound of 102(d) (0.14 g, 0.33 mmol) in THF (3 mL), pyridine(0.1 mL) and phenylmethyl 4-(chlorocarbonyl)-1-piperidinecarboxylate (0.14 g, 0.50 mmol) was stirred at 60° C. for 1 h. After cooling to RT, the solvent was removed in vacuo. Flash chromatography (2% CH₃OH/CH₂Cl₂, silica gel) gave 0.11 g (50%) of the desired compound. MS (ES+) m/z 667 (M+H)⁺.

### f) N-[2-(4-Amino-1,2,5-oxadiazol-3-yl)-1-ethyl-4-phenyl-1H-imidazo[4,5-c]pyridin-7-yl]-4-piperidinecarboxamide trifluoroacetate

To a solution of 102(e) (0.05 g, 0.075 mmol) in CH₂Cl₂ (8 mL) at −5° C. was added BBr₃ (1 mL, 1.0 M in CH₂Cl₂, 1 mmol). The reaction mixture was stirred at 0° C. for 1 h and then RT for 1 h. The mixture was diluted with MeOH (5 mL) and the solvent was evaporated in vacuo. The residue was subjected to reverse phase preparative HPLC (acetonitrile water gradient+0.1% TFA) to give the 0.018 g (33%) of the title compound. MS (ES+) m/z 433 (M+H)⁺.

### Example 103

### Preparation of N-[2-(4-amino-1,2,5-oxadiazol-3-yl)-4-phenyl-1H-imidazo[4,5-c]pyridin-7-yl]-N′-3-pyrrolidinylurea trifluoroacetate

### a) 1,1-Dimethylethyl 3-{[({2-[4-({[(1,1-dimethylethyl)oxy]carbonyl}amino)-1,2,5-oxadiazol-3-yl]-4-phenyl-1H-imidazo[4,5-c]pyridin-7-yl}amino)carbonyl]amino}-1-pyrrolidinecarboxylate

DPPA (53 uL) was added dropwise to a mixture of the compound of Example 102(c) (100 mg, 0.20 mmol) and Et₃N (37 uL) in toluene (2 mL) at RT. After 30 min. at RT, the reaction was heated at 80° C. for 30 min and then cooled to RT. 1,1-Dimethylethyl 3-amino-1-pyrrolidinecarboxylate (62 mg) was added to the resulting yellow precipitate and mixture was stirred at RT overnight, heated to 90° C. for 3 hr and cooled to RT. The reaction mixture was diluted with CH₂Cl₂, washed with 10% aq. tartaric acid, saturated NaHCO₃, brine and dried over Na₂SO₄. Removal of the solvent followed by the purification of the residue by flash chromatography (5% MeOH/CH₂Cl₂, silica gel) gave 133 mg of the desired material as a light yellow solid.

### b) N-[2-(4-Amino-1,2,5-oxadiazol-3-yl)-4-phenyl-1H-imidazo[4,5-c]pyridin-7-yl]-N′-3-pyrrolidinylurea

A solution of the compound of Example 103(a) and TFA (0.5 mL) in CH₂Cl₂ was stirred at RT for 1 hr. The solvent was removed in vacuo and the residue was azeotroped from toluene. The title compound was isolated by reverse phase HPLC (H₂O/CH₃CN/0.1% TFA) to give 40 mg of the title compound as a light brown. MS (ES+) m/z 434.4 (M+H)⁺.

### Example 104

### Preparation of 3-amino-N-[2-(4-amino-1,2,5-oxadiazol-3-yl)-4-phenyl-1H-imidazo[4,5-c]pyridin-7-yl]-1-pyrrolidinecarboxamide trifluoroacetate

The title compound was prepared in a manner analogous to the preparation of the compound of Example 103 except substituting 1,1-dimethylethyl 3-pyrrolidinylcarbamate for 1,1-dimethylethyl 3-amino-1-pyrrolidinecarboxylate. MS (ES+) m/z 434.2 (M+H)⁺.

### Example 105

### Preparation of 4-{1-ethyl-7-[(4-methyl-1-piperazinyl)methyl]-4-phenyl-1H-imidazo[4,5-c]pyridin-2-yl}-1,2,5-oxadiazol-3-amine trifluoroacetate

### a) [2-(4-Amino-1,2,5-oxadiazol-3-yl)-1-ethyl-4-phenyl-1H-imidazo[4,5-c]pyridin-7-yl]methanol

To the solution of the compound of Example 67(g) (0.50 g, 1.32 mmol) in THF (10 mL) in an ice bath was added dropwise a solution of LAH (2.64 mL, 1M in THF, 2.64 mmol). After stirring for 15 min., the reaction was quenched by sequential dropwise addition of water (100 uL), 15% NaOH (100 uL) and water (300 uL). The resulting suspension was stirred for 5 min. and then filtered. The filtrate was concentrated in vacuo to give the desired product (0.42 g, 93%). MS (ES+) m/z 337 (M+H)⁺.

### b) 4-[7-(Chloromethyl)-1-ethyl-4-phenyl-1H-imidazo[4,5-c]pyridin-2-yl]-1,2,5-oxadiazol-3-amine

The compound of Example 105(a) in CH₂Cl₂ (30 mL) and SOCl₂ (13.4 mmol) was stirred at room temperature for 2 h. DMF (0.5 mL) was added and the reaction was stirred for 1 h. A solution of 6N HCl was added and the reaction was stirred for 0.5 h. The desired material was isolated by filtration to give 0.72 g of the desired compound as a solid. MS (ES+) m/z 355 (M+H)⁺.

### c) 4-{1-Ethyl-7-[(4-methyl-1-piperazinyl)methyl]-4-phenyl-1H-imidazo[4,5-c]pyridin-2-yl}-1,2,5-oxadiazol-3-amine trifluoroacetate

A solution of the compound of Example 105(b) (50 mg, 0.13 mmol) and 1-methylpiperizine (0.52 mmol) in CH₂Cl₂ (5 mL) was stirred at RT for 18 h. The reaction mixture was diluted with CH₂Cl₂ (15 mL), washed with water, brine, and dried over Na₂SO₄. The solvent was removed in vacuo. The title compound was isolated as a solid (29 mg) by preparative reverse phase HPLC (acetonitrile water gradient+0.1% TFA). MS (ES+) m/z 419 (M+H)⁺.

### Example 106

### Preparation of N-{[2-(4-amino-1,2,5-oxadiazol-3-yl)-1-ethyl-4-phenyl-1H-imidazo[4,5-c]pyridin-7-yl]methyl}-N,1-dimethyl-4-piperidinamine

The title compound was prepared in an analogous manner to Example 105 by substituting N,1-dimethyl-4-piperidinamine for 1-methylpiperazine in step (c) and omitting the preparative reverse phase HPLC purification. MS (ES+) m/z 447.0 [M+H]⁺.

### Example 107

### Preparation of 4-(1-ethyl-7-{[3-(methylamino)-1-pyrrolidinyl]methyl}-4-phenyl-1H-imidazo[4,5-c]pyridin-2-yl)-1,2,5-oxadiazol-3-amine trifluoroacetate

The title compound was prepared in an analogous manner to Example 105 by substituting 1,1-dimethylethyl methyl(3-pyrrolidinyl)carbamate for 1-methylpiperazine in step (c). MS (ES+) m/z 419.0 [M+H]⁺.

### Example 108

### Preparation of (3-amino-2,2-dimethylpropyl){[2-(4-amino-1,2,5-oxadiazol-3-yl)-1-ethyl-4-phenyl-1H-imidazo[4,5-c]pyridin-7-yl]methyl}amine trifluoroacetate

The title compound was prepared in an analogous manner to Example 105 by substituting 2,2-dimethyl-1,3-propanediamine for 1-methylpiperazine in step (c).

MS (ES+) m/z 421.0 [M+H]⁺.

### Example 109

### Preparation of 4-(7-{[3-(dimethylamino)-1-pyrrolidinyl]methyl}-1-ethyl-4-phenyl-1H-imidazo[4,5-c]pyridin-2-yl)-1,2,5-oxadiazol-3-amine trifluoroacetate

The title compound was prepared in an analogous manner to Example 105 by substituting N,N-dimethyl-3-pyrrolidinamine for 1-methylpiperazine in step (c).

MS (ES+) m/z 433.0 [M+H]⁺.

### Example 110

### Preparation of 4-(7-{(3S)-3-amino-1-pyrrolidinyl]methyl}-1-ethyl-4-phenyl-1H-imidazo[4,5-c]pyridin-2-yl)-1,2,5-oxadiazol-3-amine

The title compound was prepared in an analogous manner to Example 105 by substituting (3R)-3-pyrrolidinamine for 1-methylpiperazine in step (c) and omitting the reverse phase preparative HPLC purification. MS (ES+) m/z 405.0 [M+H]⁺.

### Example 111

### Preparation of 4-[1-ethyl-7-(hexahydro-1H-1,4-diazepin-1-ylmethyl)-4-phenyl-1H-imidazo[4,5-c]pyridin-2-yl]-1,2,5-oxadiazol-3-amine

The title compound was prepared in an analogous manner to Example 105 by substituting hexahydro-1H-1,4-diazepine for 1-methylpiperazine in step (c) and omitting the reverse phase preparative HPLC purification. MS (ES+) m/z 419.0 [M+H]⁺.

### Example 112

### Preparation of 4-[1-ethyl-4-phenyl-7-(1-piperazinylmethyl)-1H-imidazo[4,5-c]pyridin-2-yl]-1,2,5-oxadiazol-3-amine

The title compound was prepared in an analogous manner to Example 105 by substituting piperazine for 1-methylpiperazine in step (c) and omitting the reverse phase preparative HPLC purification. MS (ES+) m/z 405.0 [M+H]⁺.

### Example 113

### Preparation of [2-(4-amino-1,2,5-oxadiazol-3-yl)-4-(3-chlorophenyl)-1-ethyl-1H-imidazo[4,5-c]pyridin-7-yl]methanol hydrochloride

### a) Ethyl 2-(4-amino-1,2,5-oxadiazol-3-yl)-4-(3-chlorophenyl)-1-ethyl-1H-imidazo[4,5-c]pyridine-7-carboxylate

The desired compound was prepared in an analogous manner to Example 67(g) by substituting ethylamine for isopropylamine in step (a) and 3-chlorophenylboronic acid for phenylboronic acid in step (b).

### b) [2-(4-amino-1,2,5-oxadiazol-3-yl)-4-(3-chlorophenyl)-1-ethyl-1H-imidazo[4,5-c]pyridin-7-yl]methanol hydrochloride

The title compound was prepared in an analogous manner to the compound of Example 105(a) substituting the compound of Example 113(a) for the compound of Example 67(g) and triturating the purified product from 3n HCl. MS (ES+) m/z 371.0 [M+H]⁺.

### Example 114

### Preparation 4-{1-ethyl-4-phenyl-7-[(3-piperidinylmethyl)oxy]-1H-imidazo[4,5-c]pyridin-2-yl}-1,2,5-oxadiazol-3-amine trifluoroacetate

### a) Ethyl (3-nitropyridin-4-yl)amine

A solution consisting of 4-ethoxy-3-nitropyridine (15.0 g, 97.3 mmol) and EtNH₂ (46.5 mL, 70% aq. solution, 584 mmol) in EtOH (30 mL) was stirred at 85° C. in a pressure vessel for 2 h. Removal of all volatiles in vacuo afforded the title compound (16.2 g, 99%). MS (ES+) m/z 168 (M+H)⁺.

### b) Ethyl (3-bromo-5-nitropyridin-4-yl)amine

A mixture of ethyl (3-nitropyridin-4-yl)amine (11.8 g, 70.0 mmol), acetic acid (140 mL), sodium acetate (28.7 g, 0.35 mol) and bromine (13.4 g, 84.0 mmol) was stirred in a pressure vessel at 100° C. for 18 h. The solvent was removed in vacuo and the residue partitioned between CH₂Cl₂ and water. The aqueous layer was made basic (pH˜8) with NaHCO₃ and further extracted with CH₂Cl₂. The combined organic extracts were washed with water, brine and dried (Na₂SO₄). The solvent was removed in vacuo. and the residue subjected to flash chromatography (20% EtOAc/hexanes, silica gel) to give 10.4 g (60%) of the desired compound. MS (ES+) m/z 246 (M+H)⁺.

### c) 5-Bromo-2-chloro-N⁴-ethyl-pyridine-3,4-diamine

To a solution of ethyl (3-bromo-5-nitropyridin-4-yl)amine (22.0 g, 89.4 mmol) in conc HCl (250 mL) was added in portions tin(II) chloride dihydrate (60.5 g, 270 mmol). The mixture was stirred at RT for 1 h and then poured onto ice (300 g). EtOAc (500 mL) was added and the mixture made basic (pH˜10) with solid NaOH. The aqueous layer was extracted with EtOAC and the combined organic layers were washed with water, brine and dried (Na₂SO₄). The solvent was removed in vacuo. and the residue subjected to flash chromatography (25% EtOAc/hexanes, silica gel) to give 17.8 g (80%) of the desired compound. MS (ES+) m/z 250 (M+H)⁺.

### d) N-(5-Bromo-2-chloro-4-ethylamino-pyridin-3-yl)-cyanoacetamide

To a solution of 5-bromo-2-chloro-N⁴-ethyl-pyridine-3,4-diamine (17.7 g, 70.9 mmol) in DMF (100 mL) at 0° C. was added cyanoacetic acid (9.06 g, 106 mmol), N-methyl morpholine (39 mL, 350 mmol) and EDCI (20.3 g, 106 mmol). The cooling bath was removed and stirring continued 3 h. The reaction was diluted with EtOAc (300 mL) and washed with water and brine. The solvent was removed in vacuo to give a solid. Recrystallization from EtOAc/hexanes afforded the desired compound (22.5 g). MS (ES+) m/z 317 (M+H)⁺.

### e) (7-Bromo-4-chloro-1-ethyl-1H-imidazo[4,5-c]pyridin-2-yl)-acetonitrile

A solution of N-(5-bromo-2-chloro-4-ethylamino-pyridin-3-yl)-cyanoacetamide (35.6 g, 112 mmol) in acetic acid (300 mL) was stirred at 90° C. for 1 h. The solvent was removed in vacuo to give the desired compound (29.5 g). This was used without further purification. MS (ES+) m/z 299 (M+H)⁺.

### f) (7-Bromo-4-chloro-1-ethyl-1H-imidazo[4,5-c]pyridin-2-yl)-hydroxyimino-acetonitrile

To a mixture of (7-bromo-4-chloro-1-ethyl-1H-imidazo[4,5-c]pyridin-2-yl)-acetonitrile (29.5 g, 98 mmol) in 2 N HCl (400 mL) at RT was added portion wise over 20 min sodium nitrite (14.0 g, 203 mmol). After stirring for an additional 30 min the resulting precipitate isolated by filtration, washed with water and dried to afford the desired compound (32 g). This was used without further purification. MS (ES+) m/z 328 (M+H)⁺.

### g) 4-(7-Bromo-4-chloro-1-ethyl-1H-imidazo[4,5-c]pyridin-2-yl)-1,2,5-oxadiazol-3-amine

A solution of the compound of Example 114(f) (98 mmol crude from previous step), Et₃N (40 mL) and 50% aq hydroxylamine (16 mL) in THF (250 mL) heated to 90° C. in a sealed pressure vessel for 1.5 h. After cooling to RT, the reaction was poured into water and extracted with EtOAC. The combined organic extracts were washed with brine and dried (Na₂SO₄). The solvent was removed in vacuo. The crude bis-oxime was dissolved in dioxane (200 mL) and Et₃N (35 mL) and heated to 150° C. in a sealed pressure vessel for 1.5 h. After allowing the reaction to cool to RT, the solvent was removed in vacuo to give a solid. Recrystallization from CH₂Cl₂ afforded the desired compound (17.3 g). MS (ES+) m/z 343 (M+H)⁺.

### h) 1,1-Dimethylethyl [4-(7-bromo-4-chloro-1-ethyl-1H-imidazo[4,5-c]pyridin-2-yl)-1,2,5-oxadiazol-3-yl]carbamate

A solution of the compound of Example 114(g) (8.50 g, 24.7 mmol), pyridine (70 mL), di-t-butyl dicarbonate (21.5 g, 98.8 mmol) and DMAP (3.01 g, 24.7 mmol) in 1,2-dichloroethane (140 mL) was stirred at 85° C. in a sealed flask for 1 h. The product mixture was partitioned between EtOAc and 1N HCl. The layers were separated and the organic extract washed with 1N HCl, brine and dried (Na₂SO₄). The solvent was removed in vacuo and the resulting solid triturated with EtOAc to afford the desired compound as beige solid (5.06 g). The mother liquor was evaporated to dryness and treated with 2% trifluoroacetic acid in CH₂Cl₂ (100 mL) for 20 h. The reaction mixture was neutralized with sat NaHCO₃, washed with brine and dried (Na₂SO₄). The solvent was removed in vacuo and the residue was subjected to flash chromatography (20% EtOAc/hexane, silica gel) to afford an additional crop of the desired compound (2.45 g). The combined yield of the desired compound was 8.55 g (78%). MS (ES+) m/z 443 (M+H)⁺.

### i) 1,1-Dimethylethyl [4-(4-chloro-1-ethyl-7-hydroxy-1H-imidazo[4,5-c]pyridin-2-yl)-1,2,5-oxadiazol-3-yl]carbamate

To a solution of the compound of Example 114(h) (2.00 g, 4.51 mmol) in THF (60 mL), at −100° C. was added n-BuLi (4.50 mL, 2.5 M in hexane, 11.3 mmol) dropwise. After five minutes, a solution of B(OMe)₃ (1.50 mL, 13.5 mmol) in THF (2 mL) was added. After 10 min., the cooling bath was removed. After 1.5 h, 3M NaOH (3 mL) and 30% w/w H₂O₂ (9 mL) were added to the reaction. After an additional 1 h, the reaction was quenched by adding EtOAc and washing sequentially with 1N HCl, H₂O and brine and then drying over Na₂SO₄. The solvent was removed in vacuo and the residue triturated with EtOAc to afford the desired compound (1.45 g). MS (ES+) m/z 381 (M+H)⁺.

### j) 1,1-Dimethylethyl [4-(1-ethyl-7-hydroxy-4-phenyl-1H-imidazo[4,5-c]pyridin-2-yl)-1,2,5-oxadiazol-3-yl]carbamate

The compound of Example 114(i) (1.40 g, 3.67 mmol), phenylboronic acid (0.90 g, 7.34 mmol) and Pd(PPh₃)₄ (0.24 g) were added to 1,4-dioxane (40 mL) and 2M Na₂CO₃ (4.04 mL, 8.1 mmol). The reaction vessel was purged with nitrogen, sealed and heated to 90° C. for 18 h. After allowing the reaction to cool to RT, the solids were removed by filtration. The filtrate was concentrated in vacuo and the residue subjected to flash chromatography (75% EtOAc/hexanes, silica gel) to give the desired compound (1.16 g). MS (ES+) m/z 423 (M+H)⁺.

### k) 4-{1-Ethyl-4-phenyl-7-[(4-piperidinylmethyl)oxy]-1H-imidazo[4,5-c]pyridin-2-yl}-1,2,5-oxadiazol-3-amine

To a suspension of polymer-bound PPh₃ (0.96 g, 1.2 mmol/g loading, 1.15 mmol) in CH₂Cl₂ (10 mL), was added 1,1-dimethylethyl 4-(hydroxymethyl)-1-piperidinecarboxylate (0.50 g, 2.30 mmol) and DEAD (0.18 mL, 1.15 mmol) dropwise. After 30 min, the suspension was cooled to 0° C. A solution of the compound of Example 1140) (0.10 g, 0.23 mmol) in CH₂Cl₂ (5 mL) was added. After 1 h at 0° C., solids were removed by filtration and exhaustively washed with CH₂Cl₂. The combined filtrates were concentrated in vacuo and the resulting residue subjected to flash chromatography (35% EtOAc/hexane, silica gel) to give the desired title compound as a di-t-butylcarbamate. MS (ES+) m/z 620 (M+H)⁺.

The di-t-butyl carbamate obtained above was dissolved in TFA(2 mL) and CH₂Cl₂ (2 mL). After 2 h, the solvent was removed in vacuo and the residue subjected to preparative reverse phase HPLC (CH₃CN/water gradient, 0.1% TFA) to give 34 mg of the title compound as a white solid. MS (ES+) m/z 420 (M+H)⁺.

### Example 115

### Preparation of 4-[7-[(4-aminobutyl)oxy]-1-ethyl-4-phenyl-1H-imidazo-[4,5-c]pyridin-2-yl]-1,2,5-oxadiazol-3-amine trifluoroacetate

The title compound was prepared in an analogous manner to Example 114 by substituting 1,1-dimethylethyl (4-hydroxybutyl)carbamate for 1,1-dimethylethyl 4-(hydroxymethyl)-1-piperidinecarboxylate in step (k). MS (ES+) m/z 394.0 [M+H]⁺

### Example 116

### Preparation of 4-{4-(3-chlorophenyl)-1-ethyl-7-[(4-piperidinylmethyl)oxy]-1H-imidazo-[4,5-c]pyridin-2-yl}-1,2,5-oxadiazol-3-amine trifluoroacetate

The title compound was prepared in an analogous manner to Example 114 by substituting 3-chlorophenylboronic acid for phenylboronic acid in step (j).

MS (ES+) m/z 454.0 [M+H]⁺

### Example 117

### Preparation of 4-[7-[(4-aminobutyl)oxy]-4-(3-chlorophenyl)-1-ethyl-1H-imidazo-[4,5-c]pyridin-2-yl]-1,2,5-oxadiazol-3-amine trifluoroacetate

The title compound was prepared in an analogous manner to Example 114 by substituting 3-chlorophenylboronic acid for phenylboronic acid in step (j) and 1,1-dimethylethyl (4-hydroxybutyl)carbamate for 1,1-dimethylethyl 4-(hydroxymethyl)-1-piperidinecarboxylate in step (k). MS (ES+) m/z 428.0 [M+H]⁺

### Example 118

### Preparation of 4-{7-[(2-aminoethyl)oxy]-1-ethyl-4-phenyl-1H-imidazo-[4,5-c]pyridin-2-yl}-1,2,5-oxadiazol-3-amine trifluoroacetate

The title compound was prepared in an analogous manner to Example 114 by substituting 1,1-dimethylethyl (4-hydroxyethyl)carbamate for 1,1-dimethylethyl 4-(hydroxymethyl)-1-piperidinecarboxylate in step (k). MS (ES+) m/z 366.0 [M+H]⁺

### Example 119

### Preparation of 4-{1-ethyl-4-phenyl-7-[(3-pyrrolidinylmethyl)oxy]-1H-imidazo-[4,5-c]pyridin-2-yl}-1,2,5-oxadiazol-3-amine trifluoroacetate

The title compound was prepared in an analogous manner to Example 114 by substituting 1,1-dimethylethyl 3-(hydroxymethyl)-1-pyrrolidinecarboxylate for 1,1-dimethylethyl 4-(hydroxymethyl)-1-piperidinecarboxylate in step (k). MS (ES+) m/z 406.0 [M+H]⁺

### Example 120

### Preparation of 4-[7-[(3-aminopropyl)oxy]-1-ethyl-4-phenyl-1H-imidazo[4,5-c]pyridin-2-yl]-1,2,5-oxadiazol-3-amine trifluoroacetate

The title compound was prepared in an analogous manner to Example 114 by substituting 1,1-dimethylethyl (4-hydroxypropyl)carbamate for 1,1-dimethylethyl 4-(hydroxymethyl)-1-piperidinecarboxylate in step (k). MS (ES+) m/z 380.0 [M+H]⁺

### Example 121

### Preparation of 4-(7-{(2S)-2-amino-3-phenylpropyl]oxy}-1-ethyl-4-phenyl-1H-imidazo-[4,5-c]pyridin-2-yl)-1,2,5-oxadiazol-3-amine trifluoroacetate

The title compound was prepared in an analogous manner to Example 114 by substituting 1,1-dimethylethyl [(1S)-2-hydroxy-1-(phenylmethyl)ethyl]carbamate for 1,1-dimethylethyl 4-(hydroxymethyl)-1-piperidinecarboxylate in step (k).

MS (ES+) m/z 456.0 [M+H]⁺

### Example 122

### Preparation of 4-[1-ethyl-4-phenyl-7-(3-piperidinyloxy)-1H-imidazo-[4,5-c]pyridin-2-yl]-1,2,5-oxadiazol-3-amine trifluoroacetate

The title compound was prepared in an analogous manner to Example 114 by substituting 1,1-dimethylethyl 4-hydroxy-1-piperidinecarboxylate for 1,1-dimethylethyl 4-(hydroxymethyl)-1-piperidinecarboxylate in step (k). MS (ES+) m/z 406.0 [M+H]⁺

### Example 123

### Preparation of 4-(1-ethyl-4-phenyl-7-{[2-(4-piperidinyl)ethyl]oxy}-1H-imidazo-[4,5-c]pyridin-2-yl)-1,2,5-oxadiazol-3-amine trifluoroacetate

The title compound was prepared in an analogous manner to Example 114 by substituting 1,1-dimethylethyl 4-(2-hydroxyethyl)-1-piperidinecarboxylate for 1,11-dimethylethyl 4-(hydroxymethyl)-1-piperidinecarboxylate in step (k). MS (ES+) m/z 434.0 [M+H]⁺

### Example 124

### Preparation of 4-(7-{f (2R)-2-amino-3-phenylpropyl]oxy}-1-ethyl-4-phenyl-1H-imidazo-[4.,5-cl pyridin-2-yl)-1,2,5-oxadiazol-3-amine trifluoroacetate

The title compound was prepared in an analogous manner to Example 114 by substituting 1,1-dimethylethyl [(1R)-2-hydroxy-1-(phenylmethyl)ethyl]carbamate for 1,1-dimethylethyl 4-(hydroxymethyl)-1-piperidinecarboxylate in step (k).

MS (ES+) m/z 456.0 [M+H]⁺

### Example 125

### Preparation of 4-(1-ethyl-7-{[2-(methylamino)ethyl]oxy}-4-phenyl-1H-imidazo-4,5-c]pyridin-2-yl)-1,2,5-oxadiazol-3-amine trifluoroacetate

The title compound was prepared in an analogous manner to Example 114 by substituting 1,1-dimethylethyl (2-hydroxyethyl)methylcarbamate for 1,1-dimethylethyl 4-(hydroxymethyl)-1-piperidinecarboxylate in step (k). MS (ES+) m/z 380.0 [M+H]⁺

### Example 126

### Preparation of 4-[1-ethyl-4-phenyl-7-([2-[(phenylmethyl)amino]ethyl]oxy)-1H-imidazo-[4,5-c]pyridin-2-yl]-1,2,5-oxadiazol-3-amine trifluoroacetate

The title compound was prepared in an analogous manner to Example 114 by substituting 1,1-dimethylethyl (2-hydroxyethyl)(phenylmethyl)carbamate for 1,1-dimethylethyl 4-(hydroxymethyl)-1-piperidinecarboxylate in step (k). MS (ES+) m/z 456.0 [M+H]⁺

### Example 127

### Preparation of 4-{1-ethyl-4-phenyl-7-[(3-piperidinylmethyl)oxy]-1H-imidazo-[4,5-c]pyridin-2-yl}-1,2,5-oxadiazol-3-amine trifluoroacetate

The title compound was prepared in an analogous manner to Example 114 by substituting 1,1-dimethylethyl 3-(hydroxymethyl)-1-piperidinecarboxylate for 1,1-dimethylethyl 4-(hydroxymethyl)-1-piperidinecarboxylate in step (k). MS (ES+) m/z 420.0 [M+H]⁺

### Example 128

### Preparation of 4-[7-[(5-aminopentyl)oxy]-1-ethyl-4-phenyl-1H-imidazo-[4,5-c]pyridin-2-yl]-1,2,5-oxadiazol-3-amine trifluoroacetate

The title compound was prepared in an analogous manner to Example 114 by substituting 1,1-dimethylethyl (5-hydroxypentyl)carbamate for 1,1-dimethylethyl 4-(hydroxymethyl)-1-piperidinecarboxylate in step (k). MS (ES+) m/z 408.0 [M+H]⁺

### Example 129

### Preparation of 4-(7-{[3-(dimethylamino)-2,2-dimethylpropyl]oxy-}1-ethyl-4-phenyl-1H-imidazo[4,5-c]pyridin-2-yl)-1,2,5-oxadiazol-3-amine trifluoroacetate

The title compound was prepared in an analogous manner to Example 114 by substituting 1,1-dimethylethyl (3-hydroxy-2,2-dimethylpropyl)carbamate for 1,1-dimethylethyl 4-(hydroxymethyl)-1-piperidinecarboxylate in step (k). MS (ES+) m/z 436.0 [M+H]⁺

### Example 130

### Preparation of 4-(7-{[2-(dimethylamino)ethyl]oxy-1-ethyl-4-phenyl-1H-imidazo-[4,5-c]pyridin-2-yl)-1,2,5-oxadiazol-3-amine trifluoroacetate

The title compound was prepared in an analogous manner to Example 114 by substituting 2-(dimethylamino)ethanol for 1,1-dimethylethyl 4-(hydroxymethyl)-1-piperidinecarboxylate in step (k). MS (ES+) m/z 394.0 [M+H]⁺

### Example 131

### Preparation of 4-(1-ethyl-4-phenyl-7-{[(2S)-2-pyrrolidinylmethyl]oxy}-1H-imidazo-[4,5-c]pyridin-2-yl)-1,2,5-oxadiazol-3-amine trifluoroacetate

The title compound was prepared in an analogous manner to Example 114 by substituting 1,1-dimethylethyl (2S)-2-(hydroxymethyl)-1-pyrrolidinecarboxylate for 1,1-dimethylethyl 4-(hydroxymethyl)-1-piperidinecarboxylate in step (k).

MS (ES+) m/z 406.0 [M+H]⁺

### Example 132

### Preparation of 4-(1-ethyl-4-phenyl-7-{[(2R)-2-pyrrolidinylmethyl]oxy}-1H-imidazo-[4,5-c]pyridin-2-yl)-1,2,5-oxadiazol-3-amine trifluoroacetate

The title compound was prepared in an analogous manner to Example 114 by substituting 1,1-dimethylethyl (2R)-2-(hydroxymethyl)-1-pyrrolidinecarboxylate for 1,1-dimethylethyl 4-(hydroxymethyl)-1-piperidinecarboxylate in step (k).

MS (ES+) m/z 406.0 [M+H]⁺

### Example 133

### Preparation of 4-[7-[(3-aminopropyl)oxy]-4-(2-chlorophenyl)-1-ethyl-1H-imidazo[4,5-c]pyridin-2-yl]-1,2,5-oxadiazol-3-amine trifluoroacetate

The title compound was prepared in an analogous manner to Example 114 by substituting 2-chlorophenylboronic acid for phenylboronic acid in step (j) and 1,1-dimethylethyl (3-hydroxypropyl)carbamate for 1,1-dimethylethyl 4-(hydroxymethyl)-1-piperidinecarboxylate in step (k). MS (ES+) m/z 414.0 [M+H]⁺

### Example 134

### Preparation of 4-[7-[(3-aminopropyl)oxy]-4-(3-chlorophenyl)-1-ethyl-1H-imidazo[4,5-c]pyridin-2-yl]-1,2,5-oxadiazol-3-amine trifluoroacetate

The title compound was prepared in an analogous manner to Example 114 by substituting 3-chlorophenylboronic acid for phenylboronic acid in step (j) and 1,1-dimethylethyl (3-hydroxypropyl)carbamate for 1,1-dimethylethyl 4-(hydroxymethyl)-1-piperidinecarboxylate in step (k). MS (ES+) m/z 414.0 [M+H]⁺

### Example 135

### Preparation of 4-[7-[(3-aminopropyl)oxy]-4-(4-chlorophenyl)-1-ethyl-1H-imidazo[4,5-c]pyridin-2-yl]-1,2,5-oxadiazol-3-amine trifluoroacetate

The title compound was prepared in an analogous manner to Example 114 by substituting 4-chlorophenylboronic acid for phenylboronic acid in step (j) and 1,1-dimethylethyl (3-hydroxypropyl)carbamate for 1,1-dimethylethyl 4-(hydroxymethyl)-1-piperidinecarboxylate in step (k). MS (ES+) m/z 414.0 [M+H]⁺

### Example 136

### Preparation of 4-[7-[(3-aminopropyl)oxy]-4-[5-chloro-2-(methyloxy)phenyl]-1-ethyl-1H-imidazo[4,5-c]pyridin-2-yl]-1,2,5-oxadiazol-3-amine trifluoroacetate

The title compound was prepared in an analogous manner to Example 114 by substituting 5-chloro-2-methoxyphenylboronic acid for phenylboronic acid in step (j) and 1,1-dimethylethyl (3-hydroxypropyl)carbamate for 1,1-dimethylethyl 4-(hydroxymethyl)-1-piperidinecarboxylate in step (k). MS (ES+) m/z 444.0 [M+H]⁺

### Example 137

### Preparation of 2-{2-(4-amino-1,2,5-oxadiazol-3-yl)-7-[(3-aminopropyl)oxy]-1-ethyl-1H-imidazo[4,5-c]pyridin-4-yl}-4-chlorophenol trifluoroacetate

The title compound was prepared in an analogous manner to Example 114 by substituting 5-chloro-2-methoxyphenylboronic acid for phenylboronic acid in step (j), 1,1-dimethylethyl (3-hydroxypropyl)carbamate for 1,1-dimethylethyl 4-(hydroxymethyl)-1-piperidinecarboxylate and BCl₃/MeOH for trifluoroacetic acid/CH₂Cl₂ in step (k). MS (ES+) m/z 430.0 [M+H]⁺

### Example 138

### Preparation of 4-[7-[(3-aminopropyl)oxy]-1-ethyl-4-(2-pyridinyl)-1H-imidazo[4,5-c]pyridin-2-yl]-1,2,5-oxadiazol-3-amine trifluoroacetate

The title compound was prepared in an analogous manner to Example 114 by substituting 2-pyridineboronic acid for phenylboronic acid in step (j) and 1,1-dimethylethyl (3-hydroxypropyl)carbamate for 1,1-dimethylethyl 4-(hydroxymethyl)-1-piperidinecarboxylate in step (k). MS (ES+) m/z 381.0 [M+H]⁺

### Example 139

### Preparation of 4-(7-{[3-(dimethylamino)propyl]oxy-1-ethyl-4-phenyl-1H-imidazo[4,5-c]pyridin-2-yl)-1,2,5-oxadiazol-3-amine trifluoroacetate

The title compound was prepared in an analogous manner to Example 114 by substituting 3-(dimethylamino)-1-propanol for 1,1-dimethylethyl 4-(hydroxymethyl)-1-piperidinecarboxylate in step (k). MS (ES+) m/z 408.0 [M+H]⁺

### Example 140

### Preparation of 4-(1-ethyl-7-{[3-(4-morpholinyl)propyl]oxy}-4-phenyl-1H-imidazo[4,5-c]pyridin-2-yl)-1,2,5-oxadiazol-3-amine trifluoroacetate

The title compound was prepared in an analogous manner to Example 114 by substituting 3-(4-morpholinyl)-1-propanol for 1,1-dimethylethyl 4-(hydroxymethyl)-1-piperidinecarboxylate in step (k). MS (ES+) m/z 450.0 [M+H]⁺

### Example 141

### Preparation of 4-(1-ethyl-7-{[3-(methylamino)propyl]oxy}-4-phenyl-1H-imidazo[4,5-c]pyridin-2-yl)-1,2,5-oxadiazol-3-amine trifluoroacetate

The title compound was prepared in an analogous manner to Example 114 by substituting 1,1-dimethylethyl (3-hydroxypropyl)methylcarbamate for 1,1-dimethylethyl 4-(hydroxymethyl)-1-piperidinecarboxylate in step (k). MS (ES+) m/z 394.0 [M+H]⁺

### Example 142

### Preparation of 4-{1-ethyl-7-[(3-hydrazinopropyl)oxy]-4-phenyl-1H-imidazo[4,5-c]pyridin-2-yl}-1,2,5-oxadiazol-3-amine trifluoroacetate

The title compound was prepared in an analogous manner to Example 114 by substituting 1,1-dimethylethyl 1-(2-hydroxyethyl)hydrazinecarboxylate for 1,1-dimethylethyl 4-(hydroxymethyl)-1-piperidinecarboxylate in step (k). MS (ES+) m/z 395.0 [M+H]⁺

### Example 143

### Preparation of 2-[(3-{[2-(4-amino-1,2,5-oxadiazol-3-yl)-1-ethyl-4-phenyl-1H-imidazo[4,5-c]pyridin-7-yl]oxy}propyl)amino]ethanol trifluoroacetate

The title compound was prepared in an analogous manner to Example 114 by substituting 1,1-dimethylethyl (3-hydroxypropyl)[2-(tetrahydro-2H-pyran-2-yloxy)ethyl]carbamate for 1,1-dimethylethyl 4-(hydroxymethyl)-1-piperidinecarboxylate in step (k). MS (ES+) m/z 424.0 [M+H]⁺

### Example 144

### Preparation of 4-(1-ethyl-7-{3-(hydroxyamino)propyl]oxy}-4-phenyl-1H-imidazo[4,5-c]pyridin-2-yl)-1,2,5-oxadiazol-3-amine trifluoroacetate

The title compound was prepared in an analogous manner to Example 114 by substituting 1,1-dimethylethyl hydroxy(3-hydroxypropyl)carbamate for 1,1-dimethylethyl 4-(hydroxymethyl)-1-piperidinecarboxylate in step (k). MS (ES+) m/z 396.0 [M+H]⁺

### Example 145

### Preparation of N-(3-{2-(4-amino-1,2,5-oxadiazol-3-yl)-7-[(3-aminopropyl)oxy]-1-ethyl-1H-imidazo[4,5-c]pyridin-4-yl}phenyl) N′-phenylurea trifluoroacetate

The title compound was prepared in an analogous manner to Example 114 by substituting (3-{[(phenylamino)carbonyl]amino}phenyl)boronic acid for phenylboronic acid in step (j) and 1,1-dimethylethyl (3-hydroxypropyl)carbamate for 1,1-dimethylethyl 4-(hydroxymethyl)-1-piperidinecarboxylate in step (k). MS (ES+) m/z 514.0 [M+H]⁺

### Example 146

### Preparation of 3-{[2-(4-amino-1,2,5-oxadiazol-3-yl)-1-ethyl-4-phenyl-1H-imidazo[4,5-c]pyridin-7-yl]oxy}-1-propanol trifluoroacetate

The title compound was prepared in an analogous manner to Example 114 by substituting 3-(tetrahydro-2H-pyran-2-yloxy)-1-propanol for 1,1-dimethylethyl 4-(hydroxymethyl)-1-piperidinecarboxylate in step (k). MS (ES+) m/z 381.0 [M+H]⁺

### Example 147

### Preparation of 4-[7-[(4-amino-2-methylbutyl)oxy]-1-ethyl-4-phenyl-1H-imidazo[4,5-c]pyridin-2-yl]-1,2,5-oxadiazol-3-amine trifluoroacetate

The title compound was prepared in an analogous manner to Example 114 by substituting 1,1-dimethylethyl (4-hydroxy-3-methylbutyl)carbamate for 1,1-dimethylethyl 4-(hydroxymethyl)-1-piperidinecarboxylate in step (k). MS (ES+) m/z 408.0 [M+H]⁺

### Example 148

### Preparation of 4-(1-ethyl-4-phenyl-7-{[2-(2-piperidinyl)ethyl]oxy}-1H-imidazo[4,5-c]pyridin-2-yl)-1,2,5-oxadiazol-3-amine trifluoroacetate

The title compound was prepared in an analogous manner to Example 114 by substituting 1,1-dimethylethyl 2-(2-hydroxyethyl)-1-piperidinecarboxylate for 1,1-dimethylethyl 4-(hydroxymethyl)-1-piperidinecarboxylate in step (k). MS (ES+) m/z 434.0 [M+H]⁺

### Example 149

### Preparation of N-(4-{[2-(4-amino-1,2,5-oxadiazol-3-yl)-1-ethyl-4-phenyl-1H-imidazo[4,5-c]pyridin-7-yl]oxy}butyl)benzenesulfonamide

The title compound was prepared in an analogous manner to Example 114 by substituting N-(4-hydroxybutyl)benzenesulfonamide for 1,1-dimethylethyl 4-(hydroxymethyl)-1-piperidinecarboxylate in step (k) and omitting the treatment with trifluoroacetic acid/CH₂Cl₂ and reverse phase HPLC. MS (ES+) m/z 534.0 [M+H]⁺

### Example 150

### Preparation of N-(4-{[2-(4-amino-1,2,5-oxadiazol-3-yl)-1-ethyl-4-phenyl-1H-imidazo[4,5-c]pyridin-7-yl]oxy}butyl)methanesulfonamide

The title compound was prepared in an analogous manner to Example 114 by substituting N-(4-hydroxybutyl)methanesulfonamide for 1,1-dimethylethyl 4-(hydroxymethyl)-1-piperidinecarboxylate in step (k) and omitting the treatment with trifluoroacetic acid/CH₂Cl₂ and reverse phase HPLC. MS (ES+) m/z 472.0 [M+H]⁺

### Example 151

### Preparation of 1-{[2-(4-amino-1,2,5-oxadiazol-3-yl)-1-ethyl-4-phenyl-1H-imidazo[4,5-c]pyridin-7-yl]oxy}-3-(4-morpholinyl)-2-propanol trifluoroacetate

### a) 2-(4-Amino-1,2,5-oxadiazol-3-yl)-1-ethyl-4-phenyl-1H-imidazo[4,5-c]pyridin-7-ol

The compound of Example 1140) (50 mg, 0.12 mmol) was stirred in 30% TFA/CH₂Cl₂ for 1 h. The solvent was removed in vacuo and the residue was azeotroped from toluene to give the desired compound. MS (ES+) m/z 323 (M+H)⁺.

### b) 4-{1-Ethyl-7-[(2-oxiranylmethyl)oxy]-4-phenyl-1H-imidazo[4,5-c]pyridin-2-yl}-1,2,5-oxadiazol-3-amine

A mixture of the compound of Example 151(a) (39 mg, 0.12 mmol), Cs₂CO₃ (195 mg, 0.60 mmol) and bromoepihydrin (22 uL, 0.25 mmol) in DMF (1 mL) was stirred at RT for 18 h. The reaction mixture was diluted with EtOAc, washed with water and dried. The solvent was removed in vacuo to give the desired compound (40 mg). MS (ES+) m/z 379 (M+H)⁺.

### c) 1-{[2-(4-Amino-1,2,5-oxadiazol-3-yl)-1-ethyl-4-phenyl-1H-imidazo[4,5-c]pyridin-7-yl]oxy}-3-(4-morpholinyl)-2-propanol trifluoroacetate

A solution of the compound of Example 151 (b) (40 mg, 0.11 mmol) and morpholine (0.6 mmol) in EtOH (1 mL) was heated at 90° C. for 15 min. The solvent was removed in vacuo and the resulting residue subjected to preparative reverse phase HPLC (acetonitrile water gradient+0.1% TFA) to give the title compound (25 mg). MS (ES+) m/z 466 (M+H)⁺.

### Example 152

### Preparation of 4-(1-ethyl-7-{[2-(4-morpholinyl)ethyl]oxy}-4-phenyl-1H-imidazo[4,5-c]pyridin-2-yl)-1,2,5-oxadiazol-3-amine trifluoroacetate

### a) 4-{7-[(2-Bromoethyl)oxy]-1-ethyl-4-phenyl-1H-imidazo[4,5-c]pyridin-2-yl}-1,2,5-oxadiazol-3-amine

A mixture of the compound of Example 151(a) (64 mg, 0.20 mmol), Cs₂CO₃ (195 mg, 0.60 mmol) and 1,2-dibromoethane (69 uL, 0.80 mmol) in DMF (1 mL) was stirred at RT for 18 h. The reaction mixture was diluted with EtOAc, washed with water and dried. The solvent was removed in vacuo to give the desired compound. MS (ES+) m/z 430 (M+H)⁺.

### b) 4-(1-Ethyl-7-{[2-(4-morpholinyl)ethyl]oxy}-4-phenyl-1H-imidazo[4,5-c]pyridin-2-yl)-1,2,5-oxadiazol-3-amine trifluoroacetate

A solution of the compound of Example 152(a) (0.20 mmol) and morpholine (1.0 mmol) in THF (2 mL) was heated at 60° C. for 20 h. The solvent was removed in vacuo and the resulting residue subjected to preparative reverse phase HPLC (acetonitrile water gradient+0.1% TFA) to give the title compound. MS (ES+) m/z 436 (M+H)⁺.

### Example 153

### Preparation of 4-(1-ethyl-4-phenyl-7-{[3-(1-piperidinyl)propyl]oxy}-1H-imidazo[4,5-c]pyridin-2-yl)-1,2,5-oxadiazol-3-amine trifluoroacetate

The title compound was prepared in an analogous manner to Example 152 by substituting 1,3-dibromopropane for 1,2-dibromoethane in step (a) and piperidine for morpholine in step (b). MS (ES+) m/z 448.0 [M+H]⁺

### Example 154

### Preparation of (2-aminoethyl)(2-{[2-(4-amino-1,2,5-oxadiazol-3-yl)-1-ethyl-4-phenyl-1H-imidazo[4,5-c]pyridin-7-yl]oxy}ethyl)amine trifluoroacetate

The title compound was prepared in an analogous manner to Example 152 by substituting 1,2-diaminoethane for morpholine in step (b). MS (ES+) m/z 409.0 [M+H]⁺

### Example 155

### Preparation of 4-(1-ethyl-4-phenyl-7-{[2-(1-piperazinyl)ethyl]oxy}-1H-imidazo[4,5-c]pyridin-2-yl)-1,2,5-oxadiazol-3-amine trifluoroacetate

The title compound was prepared in an analogous manner to Example 152 by substituting piperazine for morpholine in step (b). MS (ES+) m/z 435.0 [M+H]⁺

### Example 156

### Preparation of 4-(7-{[2-(4-acetyl-1-piperazinyl)ethyl]oxy}-1-ethyl-4-phenyl-1H-imidazo[4,5-c]pyridin-2-yl)-1,2,5-oxadiazol-3-amine trifluoroacetate

The title compound was prepared in an analogous manner to Example 152 by substituting 1-acetylpiperazine for morpholine in step (b). MS (ES+) m/z 477.0 [M+H]⁺

### Example 157

### Preparation of 4-(1-ethyl-7-{[3-(4-methyl-1-piperazinyl)propyl]oxy}-4-phenyl-1H-imidazo[4,5-c]pyridin-2-yl)-1,2,5-oxadiazol-3-amine trifluoroacetate

The title compound was prepared in an analogous manner to Example 152 by substituting 1,3-dibromopropane for 1,2-dibromoethane in step (a) and 1-methylpiperazine for morpholine in step (b). MS (ES+) m/z 463.0 [M+H]⁺

### Example 158

### Preparation of 4-(1-ethyl-4-phenyl-7-{3-(1-piperazinyl)propyl]oxy}-1H-imidazo[4,5-c]pyridin-2-yl)-1,2,5-oxadiazol-3-amine trifluoroacetate

The title compound was prepared in an analogous manner to Example 152 by substituting 1,3-dibromopropane for 1,2-dibromoethane in step (a) and piperazine for morpholine in step (b). MS (ES+) m/z 449.0 [M+H]⁺

### Example 159

### Preparation of 4-(1-ethyl-4-phenyl-7-{[2-(1-piperidinyl)ethyl]oxy}-1H-imidazo[4,5-c]pyridin-2-yl)-1,2,5-oxadiazol-3-amine trifluoroacetate

The title compound was prepared in an analogous manner to Example 152 by substituting piperidine for morpholine in step (b). MS (ES+) m/z 434.0 [M+H]⁺

### Example 160

### Preparation of (3-{[2-(4-amino-1,2,5-oxadiazol-3-yl)-1-ethyl-4-phenyl-1H-imidazo[4,5-c]pyridin-7-yl]oxy}propyl)[2-(dimethylamino)ethyl]methylamine trifluoroacetate

The title compound was prepared in an analogous manner to Example 152 by substituting 1,3-dibromopropane for 1,2-dibromoethane in step (a) and N,N,N′-trimethyl-1,2-ethanediamine for morpholine in step (b). MS (ES+) m/z 465.0 [M+H]⁺

### Example 161

### Preparation of 3-[(3-{[2-(4-amino-1,2,5-oxadiazol-3-yl)-1-ethyl-4-phenyl-1H-imidazo[4,5-c]pyridin-7-yl]oxy}propyl)amino]-1,2-propanediol trifluoroacetate

The title compound was prepared in an analogous manner to Example 152 by substituting 1,3-dibromopropane for 1,2-dibromoethane in step (a) and 3-amino-1,2-propanediol for morpholine in step (b). MS (ES+) m/z 454.0 [M+H]⁺

### Example 162

### Preparation of 4-(7-{[3-({[2,4-bis(methyloxy)phenyl]methyl}amino)propyl]oxy}-1-ethyl-4-phenyl-1H-imidazo[4,5-c]pyridin-2-yl)-1,2,5-oxadiazol-3-amine trifluoroacetate

The title compound was prepared in an analogous manner to Example 152 by substituting 1,3-dibromopropane for 1,2-dibromoethane in step (a) and 1-[2,4-bis(methyloxy)phenyl]methanamine for morpholine in step (b). MS (ES+) m/z 530.0 [M+H]⁺

### Example 163

### Preparation of (2S)-2-[(3-{[2-(4-amino-1,2,5-oxadiazol-3-yl)-1-ethyl-4-phenyl-1H-imidazo[4,5-c]pyridin-7-yl]oxy}propyl)amino]-4-methyl-1-pentanol trifluoroacetate

The title compound was prepared in an analogous manner to Example 152 by substituting 1,3-dibromopropane for 1,2-dibromoethane in step (a) and (2R)-2-amino-4-methyl-1-pentanol for morpholine in step (b). MS (ES+) m/z 480.0 [M+H]⁺

### Example 164

### Preparation of 4-{1-ethyl-4-phenyl-7-[3-(2-pyridin-4-yl-ethylamino)-propoxy-1H-imidazo[4,5-c]pyridin-2-yl}-furazan-3-ylamine trifluoroacetate

The title compound was prepared in an analogous manner to Example 152 by substituting 1,3-dibromopropane for 1,2-dibromoethane in step (a) and 2-(4-pyridinyl)ethanamine for morpholine in step (b). MS (ES+) m/z 485.6 [M+H]⁺

### Example 165

### Preparation of 4-(2-{3-[2-(4-amino-furazan-3-yl)-1-ethyl-4-phenyl-1H-imidazo[4,5-c]pyridin-7-yloxy]-propylamino}-ethyl)-benzenesulfonamide trifluoroacetate

The title compound was prepared in an analogous manner to Example 152 by substituting 1,3-dibromopropane for 1,2-dibromoethane in step (a) and 4-(2-aminoethyl)benzenesulfonamide for morpholine in step (b). MS (ES+) m/z 563.4 [M+H]⁺

### Example 166

### Preparation of 4-(1-ethyl-7-{3-[2-(1-methyl-1H-pyrrol-2-yl)-ethylamino]-propoxy}-4-phenyl-1H-imidazo[4,5-c]pyridin-2-yl)-furazan-3-ylamine trifluoroacetate

The title compound was prepared in an analogous manner to Example 152 by substituting 1,3-dibromopropane for 1,2-dibromoethane in step (a) and 2-(1-methyl-1H-pyrrol-2-yl)ethanamine for morpholine in step (b). MS (ES+) m/z 487.6 [M+H]⁺

### Example 167

### Preparation of 4-(7-{3-[2-(4-amino-phenyl)-ethylamino]-propoxy}-1-ethyl-4-phenyl-1H-imidazo[4,5-c]pyridin-2-yl)-furazan-3-ylamine trifluoroacetate

The title compound was prepared in an analogous manner to Example 152 by substituting 1,3-dibromopropane for 1,2-dibromoethane in step (a) and 4-(2-aminoethyl)aniline for morpholine in step (b). MS (ES+) m/z 499.6 [M+H]⁺

### Example 168

### Preparation of 4-(1-ethyl-7-{3-[2-(1H-imidazo-4-yl)-ethylamino]-propoxy{-4-phenyl-1H-imidazo[4,5-c]pyridin-2-yl)-furazan-3-ylamine trifluoroacetate

The title compound was prepared in an analogous manner to Example 152 by substituting 1,3-dibromopropane for 1,2-dibromoethane in step (a) and 2-(1H-imidazol-4-yl)ethanamine for morpholine in step (b). MS (ES+) m/z 474.4 [M+H]⁺

### Example 169

### Preparation of 4-{1-ethyl-7-[3-(3-imidazol-1-yl-propylamino)-propoxy]-4-phenyl-1H-imidazo[4,5-c]pyridin-2-yl}-furazan-3-ylamine trifluoroacetate

The title compound was prepared in an analogous manner to Example 152 by substituting 1,3-dibromopropane for 1,2-dibromoethane in step (a) and 2-(1H-imidazol-1-yl)ethanamine for morpholine in step (b). MS (ES+) m/z 488.4 [M+H]⁺

### Example 170

### Preparation of 4-(2-{3-[2-(4-amino-furazan-3-yl)-1-ethyl-4-phenyl-1H-imidazo[4,5-c]pyridin-7-yloxy]-propylamino}-ethyl)-phenol trifluoroacetate

The title compound was prepared in an analogous manner to Example 152 by substituting 1,3-dibromopropane for 1,2-dibromoethane in step (a) and 4-(2-aminoethyl)phenol for morpholine in step (b). MS (ES+) m/z 500.4 [M+H]⁺

### Example 171

### Preparation of 2-{3-[2-(4-amino-furazan-3-yl)-1-ethyl-4-phenyl-1H-imidazo[4,5-c]pyridin-7-yloxy]-propylamino}-1-phenyl-ethanol trifluoroacetate

The title compound was prepared in an analogous manner to Example 152 by substituting 1,3-dibromopropane for 1,2-dibromoethane in step (a) and 2-amino-1-phenylethanol for morpholine in step (b). MS (ES+) m/z 500.4 [M+H]⁺

### Example 172

### Preparation of 4-{1-ethyl-7-[3-(3-morpholin-4-yl-propylamino)-propoxy]-4-phenyl-1H-imidazo[4,5-c]pyridin-2-yl}-furazan-3-ylamine trifluoroacetate

The title compound was prepared in an analogous manner to Example 152 by substituting 1,3-dibromopropane for 1,2-dibromoethane in step (a) and 3-(4-morpholinyl)-1-propanamine for morpholine in step (b). MS (ES+) m/z 507.4 [M+H]⁺

### Example 173

### Preparation of 4-(1-ethyl-7-{3-[2-(5-methoxy-1H-indol-3-yl)-ethylamino]-propoxy}-4-phenyl-1H-imidazo[4,5-c]pyridin-2-yl)-furazan-3-ylamine trifluoroacetate

The title compound was prepared in an analogous manner to Example 152 by substituting 1,3-dibromopropane for 1,2-dibromoethane in step (a) and 2-[5-(methyloxy)-1H-indol-3-yl]ethanamine for morpholine in step (b). MS (ES+) m/z 553.6 [M+H]⁺

### Example 174

### Preparation of 4-[2-[(3-{[2-(4-amino-1,2,5-oxadiazol-3-yl)-4-(3-chlorophenyl)-1-ethyl-1H-imidazo[4,5-c]pyridin-7-yl]oxy}propyl)amino]ethyl}benzenesulfonamide trifluoroacetate

### a) 2-(4-Amino-1,2,5-oxadiazol-3-yl)-1-ethyl-4-(3-chlorophenyl)-1H-imidazo[4,5-c]pyridin-7-ol

The desired compound was prepared in an analogous manner to the compound of Example 151(a) substituting 1,1-dimethylethyl {4-[4-(3-chlorophenyl)-7-hydroxy-1H-imidazo[4,5-c]pyridin-2-yl]-1,2,5-oxadiazol-3-yl)carbamate for the compound of Example 1140).

### b) 4-[7-[(3-Bromopropyl)oxy]-4-(3-chlorophenyl)-1H-imidazo[4,5-c]pyridin-2-yl]-1,2,5-oxadiazol-3-amine

The desired compound was prepared in a manner analogous to that of Example 152(a) substituting the compound of Example 174(a) for the compound of Example 151(a) and 1,3-dibromopropane for 1,2-dibromoethane.

### c) 4-{2-[(3-{[2-(4-amino-1,2,5-oxadiazol-3-yl)-4-(3-chlorophenyl)-1-ethyl-1H-imidazo[4,5-c]pyridin-7-yl]oxy}propyl)amino]ethyl}benzenesulfonamide trifluoroacetate

The title compound was prepared in an analogous manner to Example 152(b) substituting the compound of Example 174(b) for the compound of Example 152(a) and 4-(2-aminoethyl)benzenesulfonamide for morpholine. MS (ES+) m/z 597.4 [M+H]⁺

### Example 175

### Preparation of 4-[4-(3-chlorophenyl)-1-ethyl-7-[(3-{[2-(1H-imidazol-4-yl)ethyl]amino}propyl)oxy]-1H-imidazo[4,5-c]pyridin-2-yl]-1,2,5-oxadiazol-3-amine trifluoroacetate

The title compound was prepared in an analogous manner to Example 174 by substituting 2-(1H-imidazol-4-yl)ethanamine for 4-(2-aminoethyl)benzenesulfonamide in step (c). MS (ES+) m/z 508.4 [M+H]⁺

### Example 176

### Preparation of (S)-3-{3-[2-(4-amino-furazan-3-yl)-ethyl-4-phenyl-1H-imidazo[4,5-c]pyridin-7-yloxy]-propylamino}-propane-1,2-diol trifluoroacetate

The title compound was prepared in an analogous manner to Example 152 by substituting 1,3-dibromopropane for 1,2-dibromoethane in step (a) and (2S)-3-amino-1,2-propanediol for morpholine in step (b). MS (ES+) m/z 454.4 [M+H]⁺

### Example 177

### Preparation of 4-[7-[(3-{[(3-aminophenyl)methyl]amino}propyl)oxy]-1-ethyl-4-phenyl-1H-imidazo[4,5-c]pyridin-2-yl]-1,2,5-oxadiazol-3-amine trifluoroacetate

The title compound was prepared in an analogous manner to Example 152 by substituting 1,3-dibromopropane for 1,2-dibromoethane in step (a) and 3-(aminomethyl)aniline for morpholine in step (b). MS (ES+) m/z 485.6 [M+H]⁺

### Example 178

### Preparation of 4-{1-ethyl-7-[(3-{[(5-methyl-2-pyrazinyl)methylamino]propyl)oxy]-4-phenyl-1H-imidazo[4,5-c]pyridin-2-yl}-1,2,5-oxadiazol-3-amine trifluoroacetate

The title compound was prepared in an analogous manner to Example 152 by substituting 1,3-dibromopropane for 1,2-dibromoethane in step (a) and 1-(5-methyl-2-pyrazinyl)methanamine for morpholine in step (b). MS (ES+) m/z 486.6 [M+H]⁺

### Example 179

### Preparation of 5-{[(3-{[2-(4-amino-1,2,5-oxadiazol-3-yl)-4-(3-chlorophenyl)-1-ethyl-1H-imidazo[4,5-c]pyridin-7-yl]oxy}propyl)amino]methyl}-2-methyl-4-pyrimidinamine trifluoroacetate

The title compound was prepared in an analogous manner to Example 174 by substituting 1-(2-methyl-5-pyrimidinyl)methanamine for 4-(2-aminoethyl)benzenesulfonamide in step (c). MS (ES+) m/z 535.4 [M+H]⁺

### Example 180

### Preparation of 3-[(3-{[2-(4-amino-1,2,5-oxadiazol-3-yl)-4-(3-chlorophenyl)-1-ethyl-1H-imidazo[4,5-c]pyridin-7-yl]oxy}propyl)amino]-1,2-propanediol trifluoroacetate

The title compound was prepared in an analogous manner to Example 174 by substituting 3-amino-1,2-propanediol for 4-(2-aminoethyl)benzenesulfonamide in step (c). MS (ES+) m/z 488.4 [M+H]⁺

### Example 181

### Preparation of 4-[2-[(3-{[2-(4-amino-1,2,5-oxadiazol-3-yl)-1-ethyl-4-(1H-pyrrol-2-yl)-1H-imidazo[4,5-c]pyridin-7-yl]oxy}propyl)amino]ethyl]phenol trifluoroacetate

### a) 2-(4-Amino-1,2,5-oxadiazol-3-yl)-1-ethyl-4-(1H-pyrrol-2-yl)-1H-imidazo[4,5-c]pyridin-7-ol

The desired compound was prepared in an analogous manner to the compound of Example 151(a) substituting 1,1-dimethylethyl {4-[1-ethyl-7-hydroxy-4-(1H-pyrrol-2-yl)-1H-imidazo[4,5-c]pyridin-2-yl]-1,2,5-oxadiazol-3-yl}carbamate for the compound of Example 1140).

### b) 4-[7-[(3-Bromopropyl)oxy]-1-ethyl-4-(1H-pyrrol-2-yl)-1H-imidazo[4,5-c]pyridin-2-yl]-1,2,5-oxadiazol-3-amine

The desired compound was prepared in a manner analogous to that of Example 152(a) substituting the compound of Example 181(a) for the compound of Example 151(a) and 1,3-dibromopropane for 1,2-dibromoethane.

### c) 4-{2-[(3-{[2-(4-Amino-1,2,5-oxadiazol-3-yl)-1-ethyl-4-(1H-pyrrol-2-yl)-1H-imidazo[4,5-c]pyridin-7-yl]oxy}propyl)amino]ethyl}phenol trifluoroacetetate

The title compound was prepared in an analogous manner to Example 152(b) substituting the compound of Example 181(b) for the compound of Example 152(a) and 4-(2-aminoethyl)phenol for morpholine. MS (ES+) m/z 489.4 [M+H]⁺

### Example 182

### Preparation of 4-[7-[(3-{[2-(4-aminophenyl)ethyl]amino}propyl)oxy]-1-ethyl-4-(1H-pyrrol-2-yl)-1H-imidazo[4,5-c]pyridin-2-yl]1-1,2,5-oxadiazol-3-amine trifluoroacetate

The title compound was prepared in an analogous manner to Example 181 by substituting 4-(2-aminoethyl)aniline for 4-(2-aminoethyl)phenol in step (c). MS (ES+) m/z 488.2 [M+H]⁺

### Example 183

### Preparation of 4-{[(3-{[2-(4-amino-1,2,5-oxadiazol-3-yl)-4-(3-chlorophenyl)-1-ethyl-1H-imidazo[4,5-c]pyridin-7-yl]oxy}propyl)amino]methyl}benzenesulfonamide trifluoroacetate

The title compound was prepared in an analogous manner to Example 174 by substituting 4-(aminomethyl)benzenesulfonamide for 4-(2-aminoethyl)benzenesulfonamide in step (c). MS (ES+) m/z 583.4 [M+H]⁺

### Example 184

### Preparation of 1-[(3-{[2-(4-amino-1,2,5-oxadiazol-3-yl)-4-(3-chlorophenyl)-1-ethyl-1H-imidazo[4,5-c]pyridin-7-yl]oxy}propyl)amino]-1-deoxy-D-glucitol trifluoroacetate

The title compound was prepared in an analogous manner to Example 174 by substituting 1-amino-1-deoxy-D-iditol for 4-(2-aminoethyl)benzenesulfonamide in step (c). MS (ES+) m/z 578.6 [M+H]⁺

### Example 185

### Preparation of 4-[2-[(3-{[2-(4-amino-1,2,5-oxadiazol-3-yl)-1-ethyl-4-(1H-pyrrol-2-yl)-1H-imidazo[4,5-c]pyridin-7-yl]oxy}propyl)amino]ethyl}benzenesulfonamide trifluoroacetate

The title compound was prepared in an analogous manner to Example 181 by substituting 4-(2-aminoethyl)benzenesulfonamide for 4-(2-aminoethyl)phenol in step (c). MS (ES+) m/z 552.4 [M+H]⁺

### Example 186

### Preparation of 3-{2-(4-amino-1,2,5-oxadiazol-3-Vi)-1-ethyl-4-(1H-pyrrol-2-yl)-1H-imidazo[4,5-c]pyridin-7-yl]oxy}propyl 4-(aminomethyl)benzoate trifluoroacetate

The title compound was prepared in an analogous manner to Example 181 by substituting 4-(aminomethyl)benzoic acid for 4-(2-aminoethyl)phenol in step (c).

MS (ES+) m/z 503.4 [M+H]⁺

### Example 187

### Preparation of 1-{[2-(4-amino-1,2,5-oxadiazol-3-yl)-1-ethyl-4-phenyl-1H-imidazo[4,5-c]pyridin-7-yl]oxy}-3-{[(3-aminophenyl)methyl]amino}-2-propanol trifluoroacetate

The title compound was prepared in an analogous manner to Example 151 by substituting 4-(aminomethyl)aniline for morpholine in step (c). MS (ES+) m/z 501.4 [M+H]⁺

### Example 188

### Preparation of 4-{[(3-{[2-(4-amino-1,2,5-oxadiazol-3-yl)-1-ethyl-4-phenyl-1H-imidazo[4,5-c]pyridin-7-yl]oxy}-2-hydroxypropyl)amino]methyl}benzenesulfonamide trifluoroacetate

The title compound was prepared in an analogous manner to Example 151 by substituting 4-(aminomethyl)benzenesulfonamide for morpholine in step (c). MS (ES+) m/z 565.4 [M+H]⁺

### Example 189

### Preparation of 4-{(1R)-2-[(3-{[2-(4-amino-1,2,5-oxadiazol-3-yl)-1-ethyl-4-phenyl-1H-imidazo[4,5-c]pyridin-7-yl]oxy}propyl)amino]-1-hydroxyethyl}-1,2-benzenediol trifluoroacetate

The title compound was prepared in an analogous manner to Example 152 by substituting 1,3-dibromopropane for 1,2-dibromoethane in step (a) and 4-[(1R)-2-amino-1-hydroxyethyl]-1,2-benzenediol for morpholine in step (b). MS (ES+) m/z 532.4 [M+H]⁺

### Example 190

### Preparation of 4-{(1R)-2-[(3-{[2-(4-amino-1,2,5-oxadiazol-3-yl)-1-ethyl-4-phenyl-1H-imidazo[4,5-c]pyridin-7-yl]oxy}-2-hydroxypropyl)amino]-1-hydroxyethyl}-1,2-benzenediol trifluoroacetate

The title compound was prepared in an analogous manner to Example 151 by substituting 4-[(1R)-2-amino-1-hydroxyethyl]-1,2-benzenediol for morpholine in step (c). MS (ES+) m/z 548.4 [M+H]⁺

### Example 191

### Preparation of N-(4-{[2-(4-amino-1,2,5-oxadiazol-3-yl)-4-phenyl-1H-imidazo[4,5-c]pyridin-7-yl]oxy}butyl)-1,4-benzenediamine trifluoroacetate

The title compound was prepared in an analogous manner to Example 152 by substituting 1,4-dibromobutane for 1,2-dibromoethane in step (a) and 1,4-benzenediamine for morpholine in step (b). MS (ES+) m/z 484.4 [M+H]⁺

### Example 192

### Preparation of 3-[(4-{[2-(4-amino-1,2,5-oxadiazol-3-yl)-1-ethyl-4-phenyl-1H-imidazo[4,5-c]pyridin-7-yl]oxy}butyl)amino]-1,2-propanediol trifluoroacetate

The title compound was prepared in an analogous manner to Example 152 by substituting 1,4-dibromobutane for 1,2-dibromoethane in step (a) and 3-amino-1,2-propanediol for morpholine in step (b). MS (ES+) m/z 468.0 [M+H]⁺

### Example 193

### Preparation of 4-[1-ethyl-4-phenyl-7-([4-[(3-pyridinylmethyl)amino/butyl]oxy)-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 1-(3-pyridinyl)methanamine for morpholine in step (b). MS (ES+) m/z 485.2 [M+H]⁺

### Example 194

### Preparation of 4-[2-[(4-{[2-(4-amino-1,2,5-oxadiazol-3-yl)-1-ethyl-4-phenyl-1H-imidazo[4,5-c]pyridin-7-yl]oxy}butyl)amino]ethyl]benzenesulfonamide trifluoroacetate

The title compound was prepared in an analogous manner to Example 152 by substituting 1,4-dibromobutane for 1,2-dibromoethane in step (a) and 4-(2-aminoethyl)benzenesulfonamide for morpholine in step (b). MS (ES+) m/z 577.2 [M+H]⁺

### Example 195

### Preparation of 4-[1-ethyl-4-phenyl-7-[(4-{[2-(4-pyridinyl)ethyl]amino/butyl)oxy]-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 2-(4-pyridinyl)ethanamine for morpholine in step (b). MS (ES+) m/z 499.2 [M+H]⁺

### Example 196

### Preparation of 4-[1-ethyl-4-phenyl-7-([4-[(4-pyridinylmethyl)amino/butyl]oxy)-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 1-(3-pyridinyl)methanamine for morpholine in step (b). MS (ES+) m/z 485.2 [M+H]⁺

### Example 197

### Preparation of 4-[1-ethyl-4-phenyl-7-[(4-{[2-(2-pyridinyl)ethyl]amino}butyl)oxy]-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 2-(2-pyridinyl)ethanamine for morpholine in step (b). MS (ES+) m/z 499.4 [M+H]⁺

### Example 198

### Preparation of 4-[1-ethyl-7-[(4-{[(5-methyl-2-pyrazinyl)methyl]amino}butyl)oxy]-4-phenyl-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 1-(5-methyl-2-pyrazinyl)methanamine for morpholine in step (b). MS (ES+) m/z 500.2 [M+H]⁺

### Example 199

### Preparation of 4-[1-ethyl-7-[(4-{[2-(1H-imidazol-2-yl)ethyl]amino}butyl)oxy]-4-phenyl-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 2-(1H-imidazol-2-yl)ethanamine for morpholine in step (b). MS (ES+) m/z 488.2 [M+H]⁺

### Example 200

### Preparation of 4-{[(4-{[2-(4-amino-1,2,5-oxadiazol-3-yl)-1-ethyl-4-phenyl-1H-imidazo[4,5-c]pyridin-7-yl]oxy}butyl)amino]methyl}benzenesulfonamide trifluoroacetate

The title compound was prepared in an analogous manner to Example 152 by substituting 1,4-dibromobutane for 1,2-dibromoethane in step (a) and 4-(aminomethyl)benzenesulfonamide for morpholine in step (b). MS (ES+) m/z 563.2 [M+H]⁺

### Example 201

### Preparation of N-(4-{[2-(4-amino-1,2,5-oxadiazol-3-yl)-1-ethyl-4-phenyl-1H-imidazo[4,5-c]pyridin-7-yl]oxy}butyl)-N′-2-pyrimidinyl-1,2-ethanediamine trifluoroacetate

The title compound was prepared in an analogous manner to Example 152 by substituting 1,4-dibromobutane for 1,2-dibromoethane in step (a) and N-(2-pyrimidine)ethylenediamine for morpholine in step (b). MS (ES+) m/z 515.6 [M+H]⁺

### Example 202

### Preparation of 4-{[2-(4-amino-1,2,5-oxadiazol-3-yl)-1-ethyl-4-phenyl-1H-imidazo[4,5-c]pyridin-7-yl]oxy}butyl 4-(aminomethyl)benzoate trifluoroacetate

The title compound was prepared in an analogous manner to Example 152 by substituting 1,4-dibromobutane for 1,2-dibromoethane in step (a) and 4-(aminomethyl)benzoic acid for morpholine in step (b). MS (ES+) m/z 528.2 [M+H]⁺

### Example 203

### Preparation of 4-{2-[(4-{[2-(4-amino-1,2,5-oxadiazol-3-yl)-1-ethyl-4-phenyl-1H-imidazo[4,5-c]pyridin-7-yl]oxy}butyl)amino]ethyl}-1,2-benzenediol trifluoroacetate

The title compound was prepared in an analogous manner to Example 152 by substituting 1,4-dibromobutane for 1,2-dibromoethane in step (a) and 4-(2-aminoethyl)-1,2-benzenediol for morpholine in step (b). MS (ES+) m/z 530.2 [M+H]⁺

### Example 204

### Preparation of 4-[7-[(4-{[2-(4-chlorophenyl)ethyl]amino}butyl)oxy]-1-ethyl-4-phenyl-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 2-(4-chlorophenyl)ethanamine for morpholine in step (b). MS (ES+) m/z 532.4 [M+H]⁺

### Example 205

### Preparation of 4-[2-[(4-{[2-(4-amino-1,2,5-oxadiazol-3-yl)-1-ethyl-4-phenyl-1H-imidazo[4,5-c]pyridin-7-yl]oxy}butyl)amino]ethyl]-2-fluorophenol trifluoroacetate

The title compound was prepared in an analogous manner to Example 152 by substituting 1,4-dibromobutane for 1,2-dibromoethane in step (a) and 4-(2-aminoethyl)-2-fluorophenol for morpholine in step (b). MS (ES+) m/z 532.2 [M+H]⁺

### Example 206

### Preparation of 4-[7-[(4-{[2-(4-fluorophenyl)ethyl]amino}butyl)oxy]-1-ethyl-4-phenyl-1H-imidazo[4,5-c]pyridin-2-yl]-1,2,5-oxadiazol-3-amine trifluoroacetate

The title compound was prepared in an analogous manner to Example 152 by substituting 1,4-dibromobutane for 1,2-dibromoethane in step (a) and 2-(4-fluorophenyl)ethanamine for morpholine in step (b). MS (ES+) m/z 516.4 [M+H]⁺

### Example 207

### Preparation of methyl 4-{[(4-{[2-(4-amino-1,2,5-oxadiazol-3-yl)-1-ethyl-4-phenyl-1H-imidazo[4,5-c]pyridin-7-yl]oxy}butyl)amino]methyl}benzoate trifluoroacetate

The title compound was prepared in an analogous manner to Example 152 by substituting 1,4-dibromobutane for 1,2-dibromoethane in step (a) and methyl 4-(aminomethyl)benzoate for morpholine in step (b). MS (ES+) m/z 542.2 [M+H]⁺

### Example 208

### Preparation of 4-(7-{[4-({[4-(dimethylamino)phenyl]methyl}amino)butyl]oxy}-1-ethyl-4-phenyl-1H-imidazo[4,5-c]pyridin-2-yl)-1,2,5-oxadiazol-3-amine trifluoroacetate

The title compound was prepared in an analogous manner to Example 152 by substituting 1,4-dibromobutane for 1,2-dibromoethane in step (a) and 4-(aminomethyl)-N,N-dimethylaniline for morpholine in step (b). MS (ES+) m/z 527.4 [M+H]⁺

### Example 209

### Preparation of N-(4-{[2-(4-amino-1,2,5-oxadiazol-3-yl)-1-ethyl-4-phenyl-1H-imidazo[4,5-c]pyridin-7-yl]oxy}butyl)guanidine trifluoroacetate

1H-Pyrazole-1-carboximidamide hydrochloride (19 mg, 0.13 mmol) was added to a solution of the compound of Example 115 (50 mg, 0.13 mmol) and diisopropylamine (91 uL, 0.52 mmol) in DMF (1.5 mL). After 18 h, the solvent was removed in vacuo and the residue subjected to preparative reverse phase HPLC (YMC Combiscreen ODS-A 50×20 mm, 20 mL/min, gradient, A:acetonitrile-0.1% TFA, B:water-01% TFA, 10-65% A during 7 min, UV detection at 214) to afford the title compound (35 mg). MS (ES)⁺ m/z 436.0 [M+H]⁺.

### Example 210

### Preparation of 4-{1-ethyl-7-[(1-methyl-4-piperidinyl)oxy]-4-phenyl-1H-imidazo[4,5-c]pyridin-2-yl}-1,2,5-oxadiazol-3-amine trifluoroacetate

4-Chloro-1-methylpiperidine hydrochloride (31 mg, 0.18 mmol) was added to a solution of the compound of Example 151(a) (50 mg, 0.15 mmol), Cs₂CO₃ (0.16 g, 0.48 mmol) and NaI (3 mg) in DMF (2 mL). After heating in a microwave reactor at 155° C. for 30 min., the reaction was diluted with sat. NH₄Cl and extracted EtOAc. The combined organic extracts were washed with brine, dried with sodium sulfate, and the solvent removed in vacuo. The resulting residue was subjected to preparative HPLC (YMC Combiscreen ODS-A 50×20 mm, 20 mL/min, gradient, A:acetonitrile-0.1% TFA, B:water-0.1% TFA, 10-50% A during 7 min, UV detection at 214) to afford the title compound (14 mg). MS (ES)⁺ m/z 420.0 [M+H]⁺.

### Example 211

### Preparation of 4-{[(4-{[2-(4-amino-1,2,5-oxadiazol-3-yl)-1-ethyl-4-phenyl-1H-imidazo[4,5-c]pyridin-7-yl]oxy}butyl)amino]methyl}benzoic acid trifluoroacetate

A solution of the compound of Example 207 (0.036 g., 0.065 mmol) in a mixture of methanol (10 mL.) and 1 M NaOH (2 mL.) was stirred at ambient temperature for 16 h. The solvent was removed in vacuo and the residue suspended in a mixture of water (10 mL.) and trifluoroacetic acid (0.5 mL). Solvent was removed in vacuo and the residue subjected to preparative HPLC (YMC

Combiscreen ODS-A 50×20 mm, 20 mL/min, gradient, A:acetonitrile-0.1% TFA, B:water-0.1% TFA, 10-90% A during 12 min, UV detection at 255) to give the title compound (0.027 g). MS (ES+) m/z 528.2 (M+H)⁺.

### Example 212

### Preparation of 4-[7-[(3-aminopropyl)oxy]-1-ethyl-4-(2-pyrimidinyl)-1H-imidazo[4,5-c]pyridin-2-yl]-1,2,5-oxadiazol-3-amine trifluoroacetate

A mixture of 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate (150 mg, 0.28 mmol), pyrimidin-2-yl tributyl tin (0.22 g, 0.56 mmol) and Pd(Ph₃P)₂Cl₂ (20 mg) in dioxane (5 mL) was stirred in a sealed tube at 110° C. for 8 h. Additional Pd(Ph₃P)₂Cl₂ (20 mg) was added and the temperature increased to 150° C. After 18 h, the reaction mixture was filtered and the solvent was removed in vacuo. The residue was treated with 30% TFA/CH₂Cl₂ for 30 min. The solvent was removed in vacuo and the residue azeotroped from toluene. The crude product was subjected to preparative HPLC to give the title compound (23 mg). MS (ES+) m/z 382.0 (M+H)⁺.

### Example 213

### Preparation of 4-[1-ethyl-4-phenyl-7-(1-piperazinyl)-1H-imidazo[4,5-c]pyridin-2-yl]-1,2,5-oxadiazol-3-amine trifluoroacetate

### a) 1,1-Dimethylethyl 4-{4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}-1-piperazinecarboxylate

Xantphos (10.9 mg, 0.019 mmol) and Pd₂ dba₃ (5.7 mg, 0.006 mmol) were combined in toluene (3 mL, N₂ purged) and stirred at RT for 20 min. The compound of Example 18(f) (0.14 g, 0.31 mmol), N-Boc piperazine (52 mg) and t-BuONa (75 mg) were added and the resulting mixture was stirred at 100° C. overnight. After allowing to cool to RT, the reaction was diluted with EtOAc and sequentially washed with sat. NH₄Cl, sat. Na₂CO₃, brine and then dried over Na₂SO₄. The solvent was removed in vacuo and the residue subjected to flash chromatography (3:1 hexane/EtOAc, silica gel) to give 53 mg of the desired compound as a light yellow solid.

### b) 1,1-Dimethylethyl 4-{2-[4-({[(1,1-dimethylethyl)oxy]carbonyl}amino)-1,2,5-oxadiazol-3-yl]-1-ethyl-4-phenyl-1H-imidazo[4,5-c]pyridin-7-yl}-1-piperazinecarboxylate

A mixture of the compound of Example 213(a) (53 mg, 0.097 mmol), phenylboronic acid (23.7 mg), Pd(Ph₃P)₃ (11.2 mg) and 2N Na₂CO₃ (0.21 mL) in 1,4-dioxane (0.9 mL) was stirred at 100° C. for 1 h. The reaction mixture was filtered through celite and the filter cake was rinsed with EtOAc. The combined filtrates were concentrated in vacuo and the residue was subjected to flash chromatography (3:1 hexane/EtOAc, silica gel) to give 43 mg of the desired compound as a white solid.

### c) 4-[1-Ethyl-4-phenyl-7-(1-piperazinyl)-1H-imidazo[4,5-c]pyridin-2-yl]-1,2,5-oxadiazol-3-amine trifluoroacetate

A solution of the compound of Example 213(b) and TFA (0.5 mL) in CH₂Cl₂ was stirred at RT for 1 h. The solvent was removed in vacuo and the residue azeotroped from toluene. Preparative reverse phase HPLC (H₂O/CH₃CN/0.1% TFA) gave the title compound as a light brown solid. MS (ES+) m/z 391.2 (M+H)⁺.

### Example 214

### Preparation of 4-[7-[(4-aminobutyl)oxy]-1-ethyl-4-(phenylethynyl)-1H-imidazo[4,5-c]pyridin-2-yl]-1,2,5-oxadiazol-3-aminetrifluoroacetate

### a) Bis(1,1-dimethylethyl) {4-[7-({[(1,1-dimethylethyl)oxy]carbonyl}oxy)-1-ethyl-4-(phenylethynyl)-1H-imidazo[4,5-c]pyridin-2-yl]-1,2,5-oxadiazol-3-yl}imidodicarbonate

Phenylacetylene (0.30 mL, 2.70 mmol) and diisopropylamine (0.30 mL, 2.10 mmol) were added to a solution of the compound of Example 114(i) (88 mg, 0.15 mmol) and Pd(PPh₃)₄ (50 mg, 0.043 mmol) in dioxane (3 mL). The reaction vessel was sealed and heated to 110° C. for 2 h. After cooling to RT, the solvent was removed in vacuo and the residue was subjected to flash chromatography (silica gel, 5% to 20% EtOAc/hexane) to afford the desired compound (60 mg). MS (ES)+m/z 647.0 [M+H]⁺.

### b) 2-(4-Amino-1,2,5-oxadiazol-3-yl)-1-ethyl-4-(phenylethynyl)-1H-imidazo[4,5-c]pyridin-7-ol

Trifluoracetic acid (0.40 mL) was added to a solution of the compound of Example 213(a) (54 mg, 0.08 mmol) in CH₂Cl₂ (1 mL). After 1 h, the solvent was removed in vacuo to give the desired compound (70 mg). This was used without further purification. MS (ES)⁺ m/z 347.0 [M+H]⁺.

### c) 1,1-Dimethylethyl (4-{[2-(4-amino-1,2,5-oxadiazol-3-yl)-1-ethyl-4-(phenylethynyl)-1H-imidazo[4,5-c]pyridin-7-yl]oxy}butyl)carbamate

1,1-dimethylethyl (4-iodobutyl)carbamate (62 mg, 0.21 mmol) was added to the compound of Example 213(b) (73 mg, 0.12 mmol) and Cs₂CO₃ (0.20 g, 0.6 mmol) in DMF (2 mL). The reaction vessel was sealed and heated to 65° C. for 40 min. After cooling to RT, the reaction was diluted with sat. NH₄Cl and extracted with EtOAc. The combined extracts were washed with brine and dried over Na₂SO₄. The solvent was removed in vacuo and the residue was subjected to flash chromatography (silica gel, 3% to 8% MeOH/CH₂Cl₂) to afford the desired compound (25 mg). MS (ES)⁺ m/z 518.0 [M+H]⁺.

### d) 4-[7-[(4-Aminobutyl)oxy]-1-ethyl-4-(phenylethynyl)-1H-imidazo[4,5-c]pyridin-2-yl]-1,2,5-oxadiazol-3-amine trifluoroacetate

Trifluoracetic acid (0.4 mL) was added to a solution of the compound of Example 213(c) (25 mg, 0.048 mmol) in CH₂Cl₂ (2 mL). After 1 h, the solvent was removed in vacuo and the residue was subjected to reverse phase HPLC (YMC Combiscreen ODS-A 50×20 mm, 20 mL/min, gradient, A:acetonitrile-0.1% TFA, B:water-0.1% TFA, 10-65% A during 7 min, UV detection at 214) to afford the title compound (21 mg). MS (ES)⁺ m/z 418.0 [M+H]⁺.

### Example 215

### Preparation of 4-[7-[(4-aminobutyl)oxy]-2-(4-amino-1,2,5-oxadiazol-3-yl)-1-ethyl-1H-imidazo[4,5-c]pyridin-4-yl]-2-methyl-3-butyn-2-ol trifluoroacetate

The title compound was prepared in an analogous manner to Example 214 by substituting 2-methyl-3-butyn-2-ol for phenylacetylene in step (a). MS (ES+) m/z 400.0 [M+H]⁺

### Example 216

### Preparation of 4-[7-[(4-aminobutyl)oxy]-4-(cyclopropylethynyl)-1-ethyl-1H-imidazo[4,5-c]pyridin-2-yl]-1,2,5-oxadiazol-3-amine trifluoroacetate

The title compound was prepared in an analogous manner to Example 214 by substituting ethynylcyclopropane for phenylacetylene in step (a). MS (ES+) m/z 382.0 [M+H]⁺

### Example 217

### Preparation of 4-[7-[(3-amino-1-pyrrolidinyl)carbonyl]-1-ethyl-4-(Phenylethynyl)-1H-imidazo[4,5-c]pyridin-2-yl]-1,2,5-oxadiazol-3-amine trifluoroacetate

### a) (4-{7-[1-(3-tert-Butoxycarbonylamino-pyrrolidin-1-yl)-methanoyl]-1-ethyl-4-phenylethynyl-1H-imidazo[4,5-c]pyridin-2-yl}-furazan-3-yl)-carbamic acid tert-butyl ester

A mixture of the compound of Example 18(h) (100 mg, 0.17 mmol), ethynylbenzene (42 mg, 0.41 mmol), bis(benzonitrile)palladium(II) chloride (12 mg, 0.03 mmol), copper(1) iodide (3.9 mg, 0.02 mmol), tri-tert-butylphosphine and diisopropyl amine (0.17 mL, 0.85 mmol) in dioxane (2 mL) was stirred at 80° C. for 18 h in a sealed tube. Additional ethynylbenzene (42 mg, 41 mmol) was added and stirring at 80° C. continued for 4 h. The solvent was removed in vacuo and the residue was subjected to flash chromatography (70% EtOAc/hexanes, silica gel) to afford the desired compound (60 mg). MS (ES+) m/z 643.0 (M+H)⁺.

### b) 1-[2-(4-Amino-furazan-3-yl)-1-ethyl-4-phenylethynyl-1H-imidazo[4,5-]pyridin-7-yl]-1-(3-amino-pyrrolidin-1-yl)-methanone trifluoroacetate

A solution of the compound of Example 217(a) (27 mg) in CH₂Cl₂ (2 mL) with TFA (1 mL) was stirred at room temperature for 1 h. All volatiles were removed and the residue purified by reverse phase HPLC (acetonitrile water gradient 0.1% TFA) to afford the title compound (27 mg). MS (ES+) m/z 443.0 (M+H)⁺.

### Example 218

### Preparation of 3-[3-[2-(4-Amino-furazan-3-yl)-1-ethyl-4-(1H-pyrrol-2-yl)-1H-imidazo[4,5-c]pyridin-7-yloxy]-propylamino]-propane-1,2-diol trifluoroacetate

The title compound was prepared in an analogous manner to Example 181 by substituting 3-amino-1,2-propanediol for 4-(2-aminoethyl)phenol in step (c). MS (ES+) m/z 443.2 [M+H]⁺

### Example 219

### Preparation of 2-13-[2-(4-Amino-furazan-3-yl)-1-ethyl-4-(1H-pyrrol-2-yl)-H-imidazo[4,5-c]pyridin-7-yloxy]-propylamino}-1-phenyl-ethanol trifluoroacetate

The title compound was prepared in an analogous manner to Example 181 by substituting 2-amino-1-phenylethanol for 4-(2-aminoethyl)phenol in step (c). MS (ES+) m/z 489.4 [M+H]⁺

### Example 220

### Preparation of 4-[7-[3-(5-Aminomethyl-tetrazol-1-yl)-propoxy]-1-ethyl-4-(1H-pyrrol-2-yl)-1H-imidazo[4,5-c]pyridin-2-yl]-furazan-3-ylamine

The title compound was prepared in an analogous manner to Example 181 by substituting 1-(1H-tetrazol-5-yl)methanamine for 4-(2-aminoethyl)phenol in step (c) and omitting the preparative reverse phase HPLC. MS (ES+) m/z 451.2 [M+H]⁺

### Example 221

### Preparation of 4-((R)-2-{3-[2-(4-Amino-furazan-3-yl)-1-ethyl-4-(1H-pyrrol-2-yl)-1H-imidazo[4,5-c]pyridin-7-yloxy]-propylamino}-1-hydroxy-ethyl)-benzene-1,2-diol trifluoroacetate

The title compound was prepared in an analogous manner to Example 181 by substituting 4-[(1R)-2-amino-1-hydroxyethyl]-1,2-benzenediol for 4-(2-aminoethyl)phenol in step (c). MS (ES+) m/z 521.4 [M+H]⁺

### Example 222

### Preparation of 4-[2-(4-amino-1,2,5-oxadiazol-3-yl)-7-[(3-aminopropyl)oxy]-1-ethyl-1H-imidazo[4,5-c]pyridin-4-yl]-2-methyl-3-butyn-2-ol trifluoroacetate

The title compound was prepared in an analogous manner to Example 214 by substituting 2-methyl-3-butyn-2-ol for phenylacetylene in step (a) and 1,1-dimethylethyl (3-iodopropyl)carbamate for 1,1-dimethylethyl (4-iodobutyl)carbamate in step (c). MS (ES+) m/z 386.0

### Example 223

### Preparation of 4-[2-(4-amino-1,2,5-oxadiazol-3-yl)-1-ethyl-7-[(4-piperidinylmethyl)oxy]-1H-imidazo[4,5-c]pyridin-4-yl]-2-methyl-3-butyn-2-ol trifluoroacetate

The title compound was prepared in an analogous manner to Example 214 by substituting 2-methyl-3-butyn-2-ol for phenylacetylene in step (a) and 1,1-dimethylethyl 4-(iodomethyl)-1-piperidinecarboxylate for 1,1-dimethylethyl (4-iodobutyl)carbamate in step (c). MS (ES+) m/z 426.0

### Example 224

### Preparation of 3-[2-(4-amino-1,2,5-oxadiazol-3-yl)-7-[(3-aminopropyl)oxy]-1-ethyl-1H-imidazo[4,5-c]pyridin-4-yl]-2-propyn-1-ol trifluoroacetate

The title compound was prepared in an analogous manner to Example 214 by substituting propargyl alcohol for phenylacetylene in step (a) and 1,1-dimethylethyl (3-iodopropyl)carbamate for 1,1-dimethylethyl (4-iodobutyl)carbamate in step (c). MS (ES+) m/z 358.0

### Example 225

### Preparation of 4-[7-[(3-aminopropyl)oxy]-4-(3-amino-1-propyn-1-yl)-1-ethyl-1H-imidazo[4,5-c]pyridin-2-yl]-1,2,5-oxadiazol-3-amine trifluoroacetate

The title compound was prepared in an analogous manner to Example 214 by substituting 1,1-dimethylethyl 2-propyn-1-ylcarbamate for phenylacetylene in step (a) and 1,1-dimethylethyl (3-iodopropyl)carbamate for 1,1-dimethylethyl (4-iodobutyl)carbamate in step (c). MS (ES+) m/z 357.0 [M+H]⁺

### Example 226

### Preparation of 4-[7-[(3-aminopropyl)oxy]-4-(cyclopropylethynyl)-1-ethyl-1H-imidazo[4,5-c]pyridin-2-yl]-1,2,5-oxadiazol-3-amine trifluoroacetate

The title compound was prepared in an analogous manner to Example 214 by substituting ethynylcyclopropane for phenylacetylene in step (a) and 1,1-dimethylethyl (3-iodopropyl)carbamate for 1,1-dimethylethyl (4-iodobutyl)carbamate in step (c). MS (ES+) m/z 368.0 [M+H]⁺

### Example 227

### Preparation of 4-[2-(4-amino-1,2,5-oxadiazol-3-yl)-7-[(3-aminopropyl)oxy]-1-ethyl-1H-imidazo[4,5-c]pyridin-4-yl]-3-butyn-1-ol trifluoroacetate

The title compound was prepared in an analogous manner to Example 214 by substituting 3-butyn-1-ol for phenylacetylene in step (a) and 1,1-dimethylethyl (3-iodopropyl)carbamate for 1,1-dimethylethyl (4-iodobutyl)carbamate in step (c).

MS (ES+) m/z 372.0 [M+H]⁺

### Example 228

### Preparation of 4-{7-[(3-aminopropyl)oxy]-1-ethyl-4-[3-(methyloxy)-1-propyn-1-yl]-1H-imidazo[4,5-c]pyridin-2-yl-1,2,5-oxadiazol-3-amine trifluoroacetate

The title compound was prepared in an analogous manner to Example 214 by substituting methyl 2-propyn-1-yl ether for phenylacetylene in step (a) and 1,1-dimethylethyl (3-iodopropyl)carbamate for 1,1-dimethylethyl (4-iodobutyl)carbamate in step (c). MS (ES+) m/z 372.0 [M+H]⁺

### Example 229

### Preparation of 4-[2-(4-amino-1,2,5-oxadiazol-3-yl)-7-[(3-aminopropyl)oxy]-1-ethyl-1H-imidazo[4,5-c]pyridin-4-yl]-3-butyn-2-ol trifluoroacetate

The title compound was prepared in an analogous manner to Example 214 by substituting 3-butyn-2-ol for phenylacetylene in step (a) and 1,1-dimethylethyl (3-iodopropyl)carbamate for 1,1-dimethylethyl (4-iodobutyl)carbamate in step (c).

MS (ES+) m/z 372.0 [M+H]⁺

### Example 230

### Preparation of (2S)-3-[(3-{[2-(4-amino-1,2,5-oxadiazol-3-yl)-1-(3,3-dimethylbutyl)-4-phenyl-1H-imidazo[4,5-c]pyridin-7-yl]amino}propyl)amino]-1,2-propanediol trifluoroacetate

### a) Ethyl 6-chloro-4-[(3,3-dimethylbutyl)amino]-5-nitro-3-pyridinecarboxylate

To a solution of 4,6-dichloro-5-nitro-nicotinic acid ethyl ester (1.00 g, 3.77 mmol) in CH₂Cl₂ (10 mL) at 0° C. was added Et₃N (0.58 mL, 4.15 mmol) and (3,3-dimethylbutyl)amine (0.56 mL, 4.15 mmol). After 30 min at RT, the reaction was diluted with CH₂Cl₂, washed with water and dried over MgSO₄. The solvent was removed in vacuo to provide the desired compound as a yellow solid (1.25 g). MS (ES+) m/z 330.2 (M+H)⁺.

### b) Ethyl 4-[(3,3-dimethylbutyl)amino]-5-nitro-6-phenyl-3-pyridinecarboxylate

A solution of the compound of Example 230(a) (1.25 g, 3.79 mmol), phenylboronic acid (0.92 g, 7.58 mmol), dichlorobis(triphenylphosphine)palladium(II) (0.27 g, 0.38 mmol) and 2M Na₂CO₃ (6.06 mL, 12.1 mmol) in toluene (30 mL) was heated at 110° C. in a sealed tube for 3.5 h. The reaction was allowed to cool to RT and the solvent was removed in vacuo. Flash chromatorgaphy (50:1 to 30:1 CH₂Cl₂/MeOH, silica gel) gave the desired compound as a thick yellow syrup that solidified upon standing (1.36 g).

MS (ES+) m/z 372.2 (M+H)⁺.

### c) Ethyl 5-amino-4-[(3,3-dimethylbutyl)amino]-6-phenyl-3-pyridinecarboxylate

A mixture of the compound of Example 230(b) (1.36 g, 3.67 mmol) and 10% Pd/C (0.21 g) in, absolute EtOH (70 mL) was stirred overnight at 40° C. under hydrogen gas (1 atm). The hydrogen was vented and the catalyst was removed by filtration through a pad of celite. The filter cake was washed with additional CH₂Cl₂. The solvent was removed from the combined filtrate in vacuo to afford the desired compound as a pale yellow oil that solidified upon standing (1.11 g). MS (ES+) m/z 342.4 (M+H)⁺.

### d) Ethyl 5-[(cyanoacetyl)amino]-4-[(3,3-dimethylbutyl)amino]-6-phenyl-3-pyridinecarboxylate

A solution of the compound of Example 230(c) (1.10 g, 3.22 mmol), cyanoacetic acid (0.82 g, 9.66 mmol), 4-methylmorpholine (2.12 mL, 19.3 mmol) and 1-(3-dimethylaminopropyl)-3-ethylcarbodiimide hydrochloride (1.85 g, 9.66 mmol) in dry DMF (20 mL) was stirred overnight at RT. The reaction was diluted with EtOAc and washed with water, sat NaHCO₃, water, brine and then dried over MgSO₄. The solvent was removed in vacuo to furnish the desired compound as a pale yellow solid (1.31 g). MS (ES+) m/z 409.4 (M+H)⁺.

### e) Ethyl 2-(cyanomethyl)-1-(3,3-dimethylbutyl)-4-phenyl-1H-imidazo[4,5-c]pyridine-7-carboxylate

A mixture of the compound of Example 230(d) (1.31 g, 3.21 mmol) and acetic acid (30 mL) was stirred in a sealed tube at 100° C. for 1.5 h. The solvent was removed in vacuo and the residue subjected to flash chromatography (50:1 to 30:1 CH₂Cl₂/MeOH, silica gel) to give the desired compound as a pale grey solid (1.24 g). MS (ES+) m/z 391.2 (M+H)⁺.

### f) Ethyl 2-[(E)-cyano(hydroxyimino)methyl]-1-(3,3-dimethylbutyl)-4-phenyl-1H-imidazo[4,5-c]pyridine-7-carboxylate

To a solution of the compound of Example 230(e) (1.24 g, 3.18 mmol) in glacial acetic acid (25 mL) and water (2 mL) was added NaNO₂ (0.438 g, 6.36 mmol) portionwise over 5 min. After 3 h at RT, the solvent was removed in vacuo. The residue was dissolved in CH₂Cl₂, washed with water, brine (50 mL) and dried over MgSO₄. The solvent was removed in vacuo to give the desired compound as a tan solid (1.33 g). MS (ES+) m/z 420.4 (M+H)⁺.

### g) Ethyl 2-(4-amino-1,2,5-oxadiazol-3-yl)-1-(3,3-dimethylbutyl)-4-phenyl-1H-imidazo[4,5-c]pyridine-7-carboxylate

A mixture of the compound of Example 230(f) (1.33 g, 3.17 mmol), Et₃N (4 mL, 28.7 mmol), and hydroxylamine (50 wt. % solution in water, 0.25 mL, 3.80 mmol) in dioxane (60 mL) was heated overnight in a sealed tube at 110° C. The mixture was allowed to cool to RT and the solvent was removed in vacuo. Flash chromatography (30:1 CH₂Cl₂/MeOH, silica gel) gave the desired compound as a pale yellow solid (1.13 g). MS (ES+) m/z 435.4 (M+H)⁺.

### h) 2-(4-Amino-1,2,5-oxadiazol-3-yl)-1-(3,3-dimethylbutyl)-4-phenyl-1H-imidazo[4,5-c]pyridine-7-carboxylic acid

To a solution of the compound of Example 203(g) (1.12 g, 2.58 mmol) in 2:1 MeOH/THF (45 mL) was added 6N NaOH (6.40 mL, 38.4 mmol). After stirring vigorously at RT for 1.5 h, the solvent was removed in vacuo. The residue was dissolved in water and the pH was adjusted to 76N HCl. The aqueous phase was extracted with EtOAc and the combined organic extracts were washed with brine, dried over MgSO₄ and the solvent was removed in vacuo to furnish the desired compound as a pale yellow solid (1.05 g). MS (ES+) m/z 407.4 (M+H)⁺.

### i) 1,1-Dimethylethyl [2-(4-amino-1,2,5-oxadiazol-3-yl)-1-(3,3-dimethylbutyl)-4-phenyl-1H-imidazo[4,5-c]pyridin-7-yl]carbamate

To a stirred suspension of the compound of Example 230(h) (100 mg, 0.25 mmol) in dry t-BuOH (2 mL) under argon at RT was added activated 4 Å molecular sieves, Et₃N (41 uL, 0.29 mmol) and diphenylphosphoryl azide (60 uL, 0.28 mmol). The mixture was stirred at RT for 1.5 h and then at 100° C. for 16 h. The solvent was removed in vacuo and the residue subjected to flash chromatography (30:1 CH₂Cl₂/MeOH, silica gel) to give the desired compound as a pale yellow solid (104 mg). MS (ES+) m/z 478.4 (M+H)⁺.

### j) 1,1-Dimethylethyl [2-(4-amino-1,2,5-oxadiazol-3-yl)-1-(3,3-dimethylbutyl)-4-phenyl-1H-imidazo[4,5-c]pyridin-7-yl](3-bromopropyl)carbamate

Cs₂CO₃ (60 mg, 0.183 mmol) and 1,3-dibromopropane (30 uL, 0.293 mmol) were added to a solution of the compound of Example 230(i) (35 mg, 73 umol) in dry DMF (2 mL) at 30° C. After 2 h at 30° C., the reaction was diluted with EtOAc (20 mL) and washed with water, brine and dried over MgSO₄. The solvent was removed in vacuo and the residue subjected to flash chromatography (50:1 to 30:1 CH₂Cl₂/MeOH, silica gel) gave the desired compound as a yellow solid (35 mg).

MS (ES+) m/z 598.4 (M+H)⁺.

### k) 2-(4-Amino-1,2,5-oxadiazol-3-yl)-N-(3-bromopropyl)-1-(3,3-dimethylbutyl)-4-phenyl-1H-imidazo[4,5-c]pyridin-7-amine

To a solution of the compound of Example 2300) (35 mg, 58.5 umol) in CH₂Cl₂ (3 mL) was added trifluoroacetic acid (0.5 mL). After 3 h at RT, the solvent was removed in vacuo to afford the desired compound as a yellow solid (36 mg).

MS (ES+) m/z 498.4 (M+H)⁺.

### l) (2S)-3-[(3-{[2-(4-Amino-1,2,5-oxadiazol-3-yl)-1-(3,3-dimethylbutyl)-4-phenyl-1H-imidazo[4,5-c]pyridin-7-yl]amino}propyl)amino]-1,2-propanediol trifluoroacetate

To a solution of the compound of Example 230(k) (36 mg, 58.5 umol) in DMSO (2 mL) was added (2S)-3-amino-1,2-propanediol (27 mg, 0.296 mmol), and the resultant mixture was heated at 90° C. for 0.5 h. Purification on preparative HPLC (Zorbax® SB-C18, 21.2 mm i.d. x 25 cm, 20 mL/min, gradient, A: water-0.1% trifluoroacetic acid, B: acetonitrile-0.1% trifluoroacetic acid, 10-90% acetonitrile during 12 min, UV detection at 255 nm) furnished the title compound as a yellow solid (28 mg). MS (ES+) m/z 509.4 (M+H)⁺.

### Example 231

### Preparation of N1-[2-(4-Amino-furazan-3-yl)-1-cyclopentyl-4-phenyl-1H-imidazo[4,5-c]pyridin-7-yl]-propane-1,3-diamine trifluoroacetate

The title compound was prepared in an analogous manner to Example 230 by substituting cyclopentylamine for 3,3-dimethyl-1-butanamine in step (a) and ammonia in MeOH for (2S)-3-amino-1,2-propanediol in step (I). MS (ES+) m/z 419.6 (M+H)⁺.

### Example 232

### Preparation of N-(3-Amino-benzyl)-N′-[2-(4-amino-furazan-3-yl)-1-cyclopentyl-4-phenyl-1H-imidazo[4,5-c]pyridin-7-yl]-propane-1,3-diamine trifluoroacetate

The title compound was prepared in an analogous manner to Example 230 by substituting cyclopentylamine for 3,3-dimethyl-1-butanamine in step (a) and 3-(aminomethyl)aniline for (2S)-3-amino-1,2-propanediol in step (I). MS (ES+) m/z 524.4 (M+H)⁺.

### Example 233

### Preparation of (S)-3-{3-[2-(4-Amino-furazan-3-yl)-1-cyclopentyl-4-phenyl-1H-imidazo[4,5-c]pyridin-7-ylamino]-propylamino}-propane-1,2-diol trifluoroacetate

The title compound was prepared in an analogous manner to Example 230 by substituting cyclopentylamine for 3,3-dimethyl-1-butanamine in step (a).

MS (ES+) m/z 493.4 (M+H)⁺.

### Example 234

### Preparation of N-[2-(4-amino-1,2,5-oxadiazol-3-yl)-4-(3-chlorophenyl)-1-ethyl-1H-imidazo[4,5-c]pyridin-7-yl]-1,3-propanediamine trifluoroacetate

### a) Ethyl 6-chloro-4-(ethylamino)-5-nitro-3-pyridinecarboxylate

Following the procedure of Example 230(a), except substituting ethylamine for (3,3-dimethylbutyl)amine, the desired compound was prepared. MS (ES+) m/z 274.4 (M+H)⁺.

### b) Ethyl 5-amino-6-chloro-4-(ethylamino)-3-pyridinecarboxylate

A solution of the compound of Example 234(a) (5.00 g, 18.3 mmol) in conc HCl (25 mL) at 90° C. was treated portionwise with SnCl₂ (16.6 g, 87.7 mmol). After 30 min at 90° C., the reaction was cooled to 0° C. and neutralized to pH˜7 with 50% NaOH. The mixture was diluted with CH₂Cl₂ (200 mL) and filtered through a pad of celite. The organic layer was separated, washed with brine, dried over MgSO₄. The solvent was removed in vacuo to give the desired product as a tan solid (3.32 g). MS (ES+) m/z 244.2 (M+H)⁺.

### c) Ethyl 6-chloro-5-[(cyanoacetyl)amino]-4-(ethylamino)-3-pyridinecarboxylate

Following the procedure of Example 230(d), except substituting the compound of Example 234(b) for the compound of Example 230(c), the desired compound was prepared. MS (ES+) m/z 311.2 (M+H)⁺.

### d) Ethyl 4-(3-chlorophenyl)-2-(cyanomethyl)-1-ethyl-1H-imidazo[4,5-c]pyridine-7-carboxylate

Following the procedure of Example 230(b), except substituting the compound of Example 234(c) for the compound of Example 230(a) and substituting 3-chlorophenylboronic acid for phenylboronic acid, the desired compound was prepared. MS (ES+) m/z 369.4 (M+H)⁺.

### e) Ethyl 4-(3-chlorophenyl)-2-[(E)-cyano(hydroxyimino)methyl]-1-ethyl-1H-imidazo[4,5-c]pyridine-7-carboxylate

Following the procedure of Example 230(f), except substituting the compound of Example 234(d) for the compound of Example 230(e), the desired compound was prepared. MS (ES+) m/z 398.4 (M+H)⁺.

### f) Ethyl 2-(4-amino-1,2,5-oxadiazol-3-yl)-4-(3-chlorophenyl)-1-ethyl-1H-imidazo[4,5-c]pyridine-7-carboxylate

Following the procedure of Example 230(g), except substituting the compound of Example 234(e) for the compound of Example 230(f), the desired compound was prepared. MS (ES+) m/z 413.4 (M+H)⁺.

### g) 2-(4-Amino-1,2,5-oxadiazol-3-yl)-4-(3-chlorophenyl)-1-ethyl-1H-imidazo[4,5-c]pyridine-7-carboxylic acid

Following the procedure of Example 230(h), except substituting the compound of Example 234(f) for the compound of Example 230(g), the desired compound was prepared. MS (ES+) m/z 385.2 (M+H)⁺.

### h) 1,1-Dimethylethyl [2-(4-amino-1,2,5-oxadiazol-3-yl)-4-(3-chlorophenyl)-1-ethyl-1H-imidazo[4,5-c]pyridin-7-yl]carbamate

Following the procedure of Example 230(i), except substituting the compound of Example 234(g) for the compound of Example 230(h), the desired compound was prepared (75%). MS (ES+) m/z 456.4 (M+H)⁺.

### i) 1,1-Dimethylethyl [2-(4-amino-1,2,5-oxadiazol-3-yl)-4-(3-chlorophenyl)-1-ethyl-1H-imidazo[4,5-c]pyridin-7-yl][3-({[(1,1-dimethylethyl)oxy]carbonyl}amino)propyl]carbamate

Following the procedure of Example 2300), except substituting the compound of Example 234(h) for the compound of Example 230(i) and substituting 1,1-dimethylethyl (3-bromopropyl)carbamate for 1,3-dibromopropane, the desired compound was prepared. MS (ES+) m/z 613.4 (M+H)⁺.

### j) N-[2-(4-amino-1,2,5-oxadiazol-3-yl)-4-(3-chlorophenyl)-1-ethyl-1H-imidazo[4,5-c]pyridin-7-yl]-1,3-propanediamine trifluoroacetate

Following the procedure of Example 230(k), except substituting the compound of Example 234(i) for the compound of Example 2300), the title compound was prepared. MS (ES+) m/z 413.4 (M+H)⁺.

### Example 235

### Preparation of N-(3-amino-benzyl)-N′-[2-(4-amino-furazan-3-yl)-4-(3-chloro-phenyl)-1-ethyl-1H-imidazo[4,5-c]pyridin-7-yl]-propane-1,3-diamine trifluoroacetate

### a) 1,1-Dimethylethyl [2-(4-amino-1,2,5-oxadiazol-3-yl)-4-(3-chlorophenyl)-1-ethyl-1H-imidazo[4,5-c]pyridin-7-yl](3-bromopropyl)carbamate

The desired compound was prepared in an analogous manner to the compound of Example 2300) except substituting the compound of Example 234(h) for the compound of Example 230(i).

### b) N-(3-Amino-benzyl)-N′-[2-(4-amino-furazan-3-yl)-4-(3-chloro-phenyl)-1-ethyl-1H-imidazo[4,5-c]pyridin-7-yl]-propane-1,3-diamine trifluoroacetate

The title compound was prepared in an analogous manner to steps (k) and (I) for the compound of Example 230 except substituting the compound of Example 235(a) for the compound of Example 2300) in step (k) and 3-(aminomethyl)aniline for (2S)-3-amino-1,2-propanediol in step (I). MS (ES+) m/z 518.4 [M+H]⁺.

### Example 236

### Preparation of (S)-3-[3-[2-(4-amino-furazan-3-yl)-4-(3-chloro-phenyl)-1-ethyl-1H-imidazo[4,5-c]pyridin-7-ylamino]-propylamino]-propane-1,2-diol trifluoroacetate

### a) 1,1-Dimethylethyl [2-(4-amino-1,2,5-oxadiazol-3-yl)-4-(3-chlorophenyl)-1-ethyl-1H-imidazo[4,5-c]pyridin-7-yl](3-bromopropyl)carbamate

The desired compound was prepared in an analogous manner to the compound of Example 2300) except substituting the compound of Example 234(h) for the compound of Example 230(i).

### b) N-(3-Amino-benzyl)-N′-[2-(4-amino-furazan-3-yl)-4-(3-chloro-phenyl)-1-ethyl-1H-imidazo[4,5-c]pyridin-7-yl]-propane-1,3-diamine trifluoroacetate

The title compound was prepared in an analogous manner to steps (k) and (I) for the compound of Example 230 except substituting the compound of Example 236(a) for the compound of Example 230(j) in step (k). MS (ES+) m/z 487.4 [M+H]⁺.

### Example 237

### Preparation of 1-{[2-(4-amino-1,2,5-oxadiazol-3-yl)-1-cyclopentyl-4-phenyl-1H-imidazo[4,5-c]pyridin-7-yl]amino}-3-{[(3-aminophenyl)methyl]amino}-2-propanol trifluoroacetate

The title compound was prepared in an analogous manner to Example 230 by substituting cyclopentylamine for 3,3-dimethyl-1-butanamine in step (a), epichlorohydrin for 1,3-dibromopropane in step (j) and 3-(aminomethyl)aniline for (2S)-3-amino-1,2-propanediol in step (I). MS (ES+) m/z 540.4 (M+H)⁺.

### Example 238

### 4-{[(E)-[(3-{[2-(4-amino-1,2,5-oxadiazol-3-yl)-1-ethyl-4-phenyl-1H-imidazo[4,5-c]pyridin-7-yl]oxy}propyl)amino](imino)methyl]amino}benzenesulfonamide trifluoroacetate

### a) Methyl N-[4-(aminosulfonyl)phenyl]imidothiocarbamate hydroiodide

A mixture of methyl iodide (0.43 g, 3.00 mmol), 4-thioureidobenzenesulfonamide (0.33 g, 1.44 mmol) in acetone (40 mL). was stirred 16 h at ambient temperature. The solvent was removed in vacuo and the residue triturated with ether to give the desired compound (0.51 g). MS (ES+) m/z 246.1 (M+H)⁺.

### b) 2-(4-Amino-1,2,5-oxadiazol-3-yl)-1-ethyl-4-phenyl-1H-imidazo[4,5-c]pyridin-7-ol

A mixture of the compound of Example 114(i) (0.60 g, 2.14 mmol), phenylboronic acid (0.54 g, 4.40 mmol), 2.0 M Na₂CO₃ (2.5 mL, 5.00 mmol) and Pd(PPh₃)₄ (0.30 g, 0.26 mmol) in dioxane (25 mL) was stirred 16 h at 90° C. in a sealed flask. The mixture was cooled, filtered and the filtrate concentrated in vacuo to give the crude product. Flash chromatography (50:1, then 30:1, CH₂Cl₂:MeOH, silica gel) gave the desired compound (0.43 g). MS (ES+) m/z 323.2 (M+H)⁺.

### c) 1,1-Dimethylethyl (3-{[2-(4-amino-1,2,5-oxadiazol-3-yl)-1-ethyl-4-phenyl-1H-imidazo[4,5-c]pyridin-7-yl]oxy}propyl)carbamate

A mixture of the compound of Example 238(b) (0.21 g, 0.65 mmol), 1,1-dimethylethyl (3-bromopropyl)carbamate (0.46 g, 1.96 mmol) and Cs₂CO₃ (0.64 g, 1.96 mmol) in DMF (16 mL) was stirred 16 h at ambient temperature. The solvent was removed in vacuo and the residue partitioned between EtOAc and water. The organic layer was washed with water and brine, dried (Na₂SO₄) and the solvent was removed in vacuo to give the crude product. Trituration from hexane (10 mL) gave the desired compound (0.25 g). MS (ES+) m/z 480.2 (M+H)⁺.

### d) 4-{7-[(3-Aminopropyl)oxy]-1-ethyl-4-phenyl-1H-imidazo[4,5-c]pyridin-2-yl}-1,2,5-oxadiazol-3-amine

The compound of Example 238(c)-(0.18 g, 0.37 mmol) was dissolved in a mixture of CH₂Cl₂ (10 mL) and trifluoroacetic acid (2 mL). After 1 h, the solvent was removed in vacuo and the residue partitioned between EtOAc and 0.5 M NaOH. The organic layer was washed with brine, dried (Na₂SO₄) and the solvent was removed in vacuo to give the desired compound (0.12 g). MS (ES+) m/z 380.2 (M+H)⁺.

### e) 4-{[(E)-[(3-{[2-(4-Amino-1,2,5-oxadiazol-3-yl)-1-ethyl-4-phenyl-1H-imidazo[4,5-c]pyridin-7-yl]oxy}propyl)amino](imino)methyl]amino}benzenesulfonamide trifluoroacetate

A mixture of the compound of Example 238(a) (32 mg, 0.09 mmol), the compound of Example 238(d) (30 mg, 0.08 mmol), DBU (18 mg, 0.118 mmol) and acetonitrile (2 mL) was stirred overnight at 80° C. in a sealed tube. The solvent was removed in vacuo and the residue purified by preparative reverse phase HPLC (YMC CombiPrep ODS-A, 20 mm i.d. x 50 mm, 20 mL/min, gradient, A: water-0.1% trifluoroacetic acid, B: acetonitrile-0.1% trifluoroacetic acid, 10-90% acetonitrile over 8 min, UV detection at 214 nm) to give the title compound (24 mg). MS (ES+) m/z 577.2 (M+H)⁺.

### Example 239

### Preparation of 4-{[(E)-F(3-{[2-(4-amino-1,2,5-oxadiazol-3-v)-1-ethyl-4-(1H-pyrrol-2-yl)-1H-imidazo[4,5-c]pyridin-7-yl]oxy}propyl)amino](imino)methyl]amino}benzenesulfonamide trifluoroacetate

The title compound was prepared in an analogous manner to Example 238 by substituting 2-pyrroleboronic acid for phenylboronic acid in step (b). MS (ES+) m/z 566.4 [M+H]⁺.

### Example 240

### Preparation of N-(3-{[2-(4-amino-1,2,5-oxadiazol-3-yl)-1-ethyl-4-phenyl-1H-imidazo[4,5-c]pyridin-7-yl]oxy}propyl)-N-(4-nitrophenyl)guanidine trifluoroacetate

The title compound was prepared in an analogous manner to Example 238 by substituting N-(4-hydroxyphenyl)thiourea for 4-thioureidobenzenesulfonamide in step (a). MS (ES+) m/z 543.2 [M+H]⁺.

### Example 241

### Preparation of N-(3-{[2-(4-amino-1,2,5-oxadiazol-3-yl)-1-ethyl-4-phenyl-1H-imidazo[4,5-c]pyridin-7-yl]oxy}propyl)-N′-(4-hydroxyphenyl)guanidine trifluoroacetate

The title compound was prepared in an analogous manner to Example 238 by substituting N-(4-nitrophenyl)thiourea for 4-thioureidobenzenesulfonamide in step (a). MS (ES+) m/z 514.4 [M+H]⁺.

### Example 242

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N-(4-chlorophenyl)urea trifluoroacetate

### a) [3-({[(4-Chlorophenyl)amino]carbonyl}amino)phenyl]boronic acid

4-chlorophenyl isocyanate (0.52 g, 3.50 mmol) was added to (3-aminophenyl)boronic acid (0.48 g, 3.50 mmol) in THF (25 mL) at 0° C. After 5 min, the reaction was allowed to warm to RT. After 4 h, half of the solvent was removed in vacuo and reaction was poured into water (50 mL). The precipitate was collected by filtration and washed with water and Et₂O. The solid was dried under vacuum for 2 h at 40° C. to afford the desired compound (0.80 g). MS (ES)⁺ m/e 290.0 [M+H]⁺.

### b) 1,1-Dimethylethyl [3-({2-(4-amino-1,2,5-oxadiazol-3-yl)-4-[3-({[(4-chlorophenyl)amino]carbonyl}amino)phenyl]-1-ethyl-1H-imidazo[4,5-c]pyridin-7-yl}oxy)propyl]carbamate

To 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate (85 mg, 0.20 mmol) and the compound of Example 242(a) (0.12 g, 0.40 mmol) in dioxane (3 mL) was added Pd(PPh₃)₄ (25 mg, 0.02, mmol) and 2M Na₂CO₃ (0.3 mL). The reaction vessel was purged with argon then sealed and heated at 95° C. for 16 h. The solvent was removed in vacuo and the residue was subjected to flash chromatography (0.5% to 2% MeOH/CH₂Cl₂, silica gel) to afford the desired compound as a solid (0.12 g).

MS (ES)⁺ m/e 649.0 [M+H]⁺.

### c) N-(3-{2-(4-Amino-1,2,5-oxadiazol-3-yl)-7-[(3-aminopropyl)oxy]-1-ethyl-1H-imidazo[4,5-c]pyridin-4-yl}phenyl)-1-(4-chlorophenyl)urea trifluoroacetate

Trifluoroacetic acid (1 mL) was added to a solution of the compound of Example 242(b) (0.12 g, 0.20 mmol) in CH₂Cl₂ (3 mL). After 2 h at RT, the solvent was removed in vacuo and the residue subjected to reverse phase HPLC (YMC Combiscreen ODS-A 57×30 mm, 25 mL/min, gradient, A:acetonitrile-0.1% TFA, B:water-0.1% TFA, 8-75% A during 10 min, UV detection at 214) to afford the title compound (80 mg). MS (ES)⁺ m/e 548.0 [M+H]⁺.

### Example 243

### Preparation of N-(3-{2-(4-amino-1,2,5-oxadiazol-3-yl)-7-[(3-aminopropyl)oxy]-1-ethyl-1H-imidazo[4,5-c]pyridin-4-yl}phenyl)-N′-methylurea trifluoroacetate

The title compound was prepared in an analogous manner to Example 242 by substituting methylisocyanate for 4-chlorophenylisocyanate in step (a).

MS (ES+) m/z 452.0 [M+H]⁺.

### Example 244

### Preparation of N-(3-{2-(4-amino-1,2,5-oxadiazol-3-yl)-7-[(3-aminopropyl)oxy]-1-ethyl-1H-imidazo[4,5-c]pyridin-4-yl}phenyl)-N′-(phenylmethyl)urea trifluoroacetate

The title compound was prepared in an analogous manner to Example 242 by substituting benzylisocyanate for 4-chlorophenylisocyanate in step (a).

MS (ES+) m/z 528.0 [M+H]⁺.

### Example 245

### Preparation of N-(3-[2-(4-amino-1,2,5-oxadiazol-3-yl)-1-ethyl-7-[(4-Piperidinylmethyl)oxy]-1H-imidazo[4,5-c]pyridin-4-yl]phenyl)-N′-ethylurea

The title compound was prepared in an analogous manner to Example 242 by substituting ethylisocyanate for 4-chlorophenylisocyanate in step (a) and 1,1-dimethylethyl 4-({[2-(4-amino-1,2,5-oxadiazol-3-yl)-4-chloro-1-ethyl-1H-imidazo[4,5-c]pyridin-7-yl]oxy}methyl)-1-piperidinecarboxylate for 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate in step (b). MS (ES+) m/z 506.0 [M+H]⁺.

### Example 246

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-ethylurea trifluoroacetate

The title compound was prepared in an analogous manner to Example 242 by substituting ethylisocyanate for 4-chlorophenylisocyanate in step (a). MS (ES+) m/z 466.0 [M+H]⁺.

### Example 247

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-(1-methylethyl)urea trifluoroacetate

The title compound was prepared in an analogous manner to Example 242 by substituting isopropylisocyanate for 4-chlorophenylisocyanate in step (a).

MS (ES+) m/z 480.0 [M+H]⁺.

### Example 248

### Preparation of N-(3-{2-(4-amino-1,2,5-oxadiazol-3-yl)-7-[(3-aminopropyl)oxy]-1-ethyl-1H-imidazo[4,5-c]pyridin-4-yl}phenyl)-N′-[3-(trifluoromethyl)phenyl]urea trifluoroacetate

The title compound was prepared in an analogous manner to Example 242 by substituting 3-(trifluoromethyl)phenylisocyanate for 4-chlorophenylisocyanate in step (a). MS (ES+) m/z 582.0 [M+H]⁺.

### Example 249

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-[4-(trifluoromethyl)phenyl]urea trifluoroacetate

The title compound was prepared in an analogous manner to Example 242 by substituting 4-(trifluoromethyl)phenylisocyanate for 4-chlorophenylisocyanate in step (a). MS (ES+) m/z 582.0 [M+H]⁺.

### Example 250

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-[3-(methyloxy)phenyl]urea trifluoroacetate

The title compound was prepared in an analogous manner to Example 242 by substituting 3-(methoxy)phenylisocyanate for 4-chlorophenylisocyanate in step (a). MS (ES+) m/z 544.0 [M+H]⁺.

### Example 251

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-[4-(methyloxy)phenyl]urea

The title compound was prepared in an analogous manner to Example 242 by substituting 4-(methoxy)phenylisocyanate for 4-chlorophenylisocyanate in step (a). MS (ES+) m/z 544.0 [M+H]⁺.

### Example 252

### Preparation of N-(3-{2-(4-amino-1,2,5-oxadiazol-3-yl)-1-ethyl-7-[(4-piperidinylmethyl)oxy]-1H-imidazo[4,5-c]pyridin-4-yl}phenyl)-N′-(phenylmethyl)urea trifluoroacetate

The title compound was prepared in an analogous manner to Example 242 by substituting benzylisocyanate for 4-chlorophenylisocyanate in step (a) and 1,1-dimethylethyl 4-({[2-(4-amino-1,2,5-oxadiazol-3-yl)-4-chloro-1-ethyl-1H-imidazo[4,5-c]pyridin-7-yl]oxy}methyl)-1-piperidinecarboxylate for 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate in step (b). MS (ES+) m/z 568.0 [M+H]⁺.

### Example 253

### Preparation of N-(3-[2-(4-amino-1,2,5-oxadiazol-3-yl)-1-ethyl-7-[(4-piperidinylmethyl)oxy]-1H-imidazo[4,5-c]pyridin-4-yl]phenyl)-N′-(3-chlorophenyl)urea trifluoroacetate

The title compound was prepared in an analogous manner to Example 242 by substituting 3-chlorophenylisocyanate for 4-chlorophenylisocyanate in step (a) and 1,1-dimethylethyl 4-({[2-(4-amino-1,2,5-oxadiazol-3-yl)-4-chloro-1-ethyl-1H-imidazo[4,5-c]pyridin-7-yl]oxy}methyl)-1-piperidinecarboxylate for 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate in step (b). MS (ES+) m/z 588.0 [M+H]⁺.

### Example 254

### Preparation of N-(3-{2-(4-amino-1,2,5-oxadiazol-3-yl)-1-ethyl-7-[(4-piperidinylmethyl)oxy]-1H-imidazo[4,5-c]pyridin-4-yl}phenyl)-N′-[3 (methyloxy)phenyl]urea trifluoroacetate

The title compound was prepared in an analogous manner to Example 242 by substituting 3-methoxyphenylisocyanate for 4-chlorophenylisocyanate in step (a) and 1,1-dimethylethyl 4-({[2-(4-amino-1,2,5-oxadiazol-3-yl)-4-chloro-1-ethyl-1H-imidazo[4,5-c]pyridin-7-yl]oxy}methyl)-1-piperidinecarboxylate for 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate in step (b). MS (ES+) m/z 584.0 [M+H]⁺.

### Example 255

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-[2-(methyloxy)phenyl]urea trifluoroacetate

The title compound was prepared in an analogous manner to Example 242 by substituting 2-(methoxy)phenylisocyanate for 4-chlorophenylisocyanate in step (a). MS (ES+) m/z 544.0 [M+H]⁺.

### Example 256

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-(2,3-dihydro-1H-inden-5-yl)urea trifluoroacetate

The title compound was prepared in an analogous manner to Example 242 by substituting 5-isocyanato-2,3-dihydro-1H-indene for 4-chlorophenylisocyanate in step (a). MS (ES+) m/z 554.0 [M+H]⁺.

### Example 257

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-(2-chlorophenyl)urea trifluoroacetate

The title compound was prepared in an analogous manner to Example 242 by substituting 2-chlorophenylisocyanate for 4-chlorophenylisocyanate in step (a).

MS (ES+) m/z 548.0 [M+H]⁺.

### Example 258

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-(3-chlorophenyl)urea trifluoroacetate

The title compound was prepared in an analogous manner to Example 242 by substituting 3-chlorophenylisocyanate for 4-chlorophenylisocyanate in step (a).

MS (ES+) m/z 548.0 [M+H]⁺.

### Example 259

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-(4-cyanophenyl)urea trifluoroacetate

The title compound was prepared in an analogous manner to Example 242 by substituting 4-cyanophenylisocyanate for 4-chlorophenylisocyanate in step (a).

MS (ES+) m/z 540.0 [M+H]⁺.

### Example 260

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-N′-(3-cyanophenyl)urea trifluoroacetate

The title compound was prepared in an analogous manner to Example 242 by substituting 3-cyanophenylisocyanate for 4-chlorophenylisocyanate in step (a).

MS (ES+) m/z 539.0 [M+H]⁺.

### Example 261

### Preparation of N-(3-{2-(4-amino-1,2,5-oxadiazol-3-yl)-7-[(3-aminopropyl)oxy]-1-ethyl-1H-imidazo[4,5-c]pyridin-4-yl}phenyl)-N′-cyclohexylurea trifluoroacetate

The title compound was prepared in an analogous manner to Example 242 by substituting cyclohexylisocyanate for 4-chlorophenylisocyanate in step (a).

MS (ES+) m/z 520.0 [M+H]⁺.

### Example 262

### Preparation of N-(3-{2-(4-amino-1,2,5-oxadiazol-3-yl)-1-ethyl-7-[(4-piperidinylmethyl)oxy]-1H-imidazo[4,5-c]pyridin-4-yl}phenyl)acetamide trifluoroacetate

The title compound was prepared in an analogous manner to Example 242 by substituting acetyl chloride for 4-chlorophenylisocyanate in step (a) and 1,1-dimethylethyl 4-({[2-(4-amino-1,2,5-oxadiazol-3-yl)-4-chloro-1-ethyl-1H-imidazo[4,5-c]pyridin-7-yl]oxy}methyl)-1-piperidinecarboxylate for 1,1-dimethylethyl [3-({4-chloro-2-[4-({[(1,1-dimethylethyl)oxy]carbonyl}amino)-1,2,5-oxadiazol-3-yl]-1-ethyl-1H-imidazo[4,5-c]pyridin-7-yl}oxy)propyl]carbamate in step (b). MS (ES+) m/z 477.0 [M+H]⁺.

### Example 263

### Preparation of ethyl 3-({[(3-{2-(4-amino-1,2,5-oxadiazol-3-yl)-7-[(3-aminopropyl)oxy]-1-ethyl-1H-imidazo[4,5-c]pyridin-4-yl}phenyl)amino]carbonyl}amino)benzoate trifluoroacetate

The title compound was prepared in an analogous manner to Example 242 by substituting ethyl 4-isocyanatobenzoate for 4-chlorophenylisocyanate in step (a).

MS (ES+) m/z 586.0 [M+H]⁺.

### Example 264

### Preparation of N-(3-[2-(4-amino-1,2,5-oxadiazol-3-yl)-7-[(3-aminopropyl)oxy]-1-ethyl-1H-imidazo[4,5-c]pyridin-4-yl]phenyl)-3-(methyloxy)propanamide trifluoroacetate

The title compound was prepared in an analogous manner to Example 242 by substituting 3-(methyloxy)propanoyl chloride for 4-chlorophenylisocyanate in step (a). MS (ES+) m/z 481.0 [M+H]⁺.

### Example 265

### Preparation of 4-(2-(4-amino-1,2,5-oxadiazol-3-yl)-1-ethyl-7-{[3-({2-[4-(methyloxy)phenyl]ethylamino)Propyl]oxy}-1H-imidazo[4,5-c]pyridin-4-yl)-2-methyl-3-butyn-2-ol

### (a) 2-(4-Amino-1,2,5-oxadiazol-3-yl)-4-chloro-1-ethyl-1H-imidazo[4,5-c]pyridin-7-ol

A solution of the compound of Example 114(g) (2.0 g, 5.8 mmol) in THF (270 mL) was cooled to −100° C. under an atmosphere of nitrogen. n-Butyl lithium (7.2 mL, 18 mmol, 2.5 M in hexanes) at −78° C. was added over 4 minutes using a syringe pump. After an additional 3 min at −100° C. trimethyl borate (2.1 mL, 19 mmol) was added. The cooling bath was removed and the mixture was allowed to warm to RT. After 3 h, a solution of 30% aqueous hydrogen peroxide (13 mL) in 3M NaOH (4.3 mL) was added. After an additional 45 min, the reaction was quenched by partitioning between EtOAc and 1N HCl. The aqueous layer was extracted with additional EtAOc and the combined organic extracts were washed with water, brine, and dried over Na₂SO₄. The solvent was removed in vacuo and the residue was triturated with 3% MeOH/CH₂Cl₂ to give the desired material as a pale yellow solid (0.96 g). MS (ES+) m/z 281.0 [M+H]⁺.

### (b) 4-(2-(4-Amino-1,2,5-oxadiazol-3-yl)-1-ethyl-7-{[3-({2-[4-(methyloxy)phenyl]ethyl}amino)propyl]oxy}-1H-imidazo[4,5-c]pyridin-4-yl)-2-methyl-3-butyn-2-ol

Anhydrous Cs₂CO₃ (1.4 g, 4.2 mmol) was added to a solution of the compound of Example 265(a) (1.0 g, 3.6 mmol) in DMF (40 mL) at RT. After 5 min., 1,3-dibromopropane (2.9 g, 14 mmol) was added and the mixture was heated to 60° C. for 3.5 h. The mixture was cooled to RT, filtered through celite and the filter cake rinsed with EtAOc. The combined filtrate was concentrated to a brown residue, which was dissolved in DMF (40 mL). Et3N (1.9 mL, 14 mmol) and 2-[4-(methyloxy)phenyl]ethanamine (1.9 mL, 13 mmol) were added and the mixture was heated to 60° C. After 30 min., the reaction was cooled to RT and quenched by partitioning between EtOAc and water. The aqueous layer was extracted with additional EtOAc, and the combined extracts were washed with water and brine, dried over MgSO₄. The solvent was removed in vacuo to give a brown solid. Trituration with Et₂O gave the title compound as a pale yellow solid (1.4 g). MS (ES+) m/z 472.0 [M+H]⁺.

### Example 266

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| 4-(4-Phenyl-1-piperidin-4-yl-1H-imidazo[4,5- | 25 mg |
| c]pyridin-2-yl)-furazan-3-ylamine | |
| Lactose | 55 mg |
| Talc | 16 mg |
| Magnesium Stearate | 4 mg |

### Example 267

### Injectable Parenteral Composition

An injectable form for administering the present invention is produced by stirring 1.5% by weight of 1-[2-(4-Aminofurazan-3-yl)-1-ethyl-4-phenyl-1-H-imidazo[4,5-c]pyridin-7-yl]-1-(3-aminopyrrolidin-1-yl)methanone in 10% by volume propylene glycol in water.

### Example 268

### Tablet Composition

The sucrose, calcium sulfate dihydrate and an Akt inhibitor as shown in Table II below, are mixed and granulated in the proportions shown with a 10% gelatin solution. The wet granules are screened, dried, mixed with the starch, talc and stearic acid; screened and compressed into a tablet.

TABLE II

| INGREDIENTS | AMOUNTS | |
|---|---|---|
| 2-(4-amino-1,2,5-oxadiazol-3-yl)-4-(3-chlorophenyl)-1- | 20 | mg |
| (cyclopropylmethyl)-N-[3-(dimethylamino)propyl]-1H- | | |
| imidazo[4,5-c]pyridine-7-carboxamide | | |
| calcium sulfate dihydrate | 30 | mg |
| sucrose | 4 | mg |
| starch | 2 | mg |
| talc | 1 | mg |
| stearic acid | 0.5 | mg |

While the preferred embodiments of the invention are illustrated by the above, it is to be understood that the invention is not limited to the precise instructions herein disclosed and that the right to all modifications coming within the scope of the following claims is reserved.

What is claimed is:

1. A compound of Formula (I): 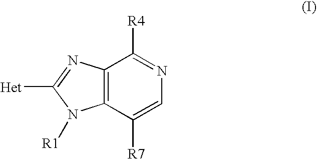
wherein:
Het is selected from the group consisting of: 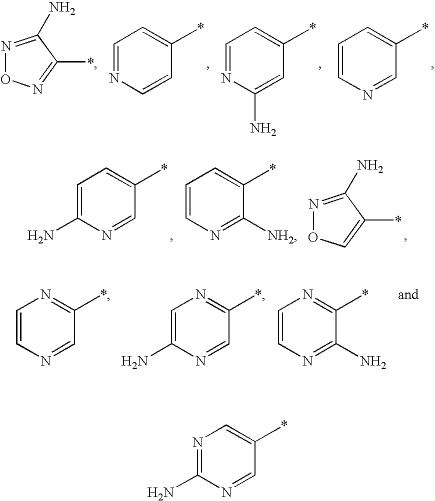
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 4 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶, —S(O)ₙR² and protected —OH,
where n is 0-2,
R² is selected from hydrogen, alkyl, cycloalkyl, C₁-C₁₂aryl, substituted alkyl, substituted cycloalkyl and substituted C₁-C₁₂aryl, and
R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from hydrogen, —C(O)NR⁹R¹⁰, —(CH₂)ₙNR⁹R¹⁰, —SO₂NR⁹R¹⁰, —(CH₂)ₙOR⁸, —O—(CH₂)ₘNR⁹R¹⁰ and —N—(CH₂)ₘNR⁹R¹⁰,
where n is 0-2,
m is 1-6, where the carbon chain formed by m is optionally substituted,
R⁸ is alkyl, cycloalkyl, cycloalkyl containing from 1 to 4 heteroatoms, and aryl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 4 heteroatoms, substituted cycloalkyl containing from 1 to 4 heteroatoms, substituted cycloalkyl, halogen, aryl, substituted aryl and protected —OH,
where R² and R³ are independently hydrogen, alkyl, cycloalkyl, C₁-C₁₂aryl, substituted alkyl, substituted cycloalkyl and substituted C₁-C₁₂aryl, and n is 0-2,
R⁹ and R¹⁰ are independently hydrogen, cycloalkyl, cycloalkyl containing from 1 to 4 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 4 heteroatoms, substituted cycloalkyl, halogen, aryl, substituted aryl and protected —OH,
or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine.

2. A pharmaceutically acceptable salt, hydrate, solvate or pro-drug of a compound of Formula (I), as described in claim 1.

3. A compound of claim 1 represented by the following Formula (II): 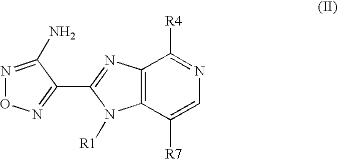
wherein:
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 4 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶, —S(O)ₙR² and protected —OH, where n is 0-2,
R² is selected from hydrogen, alkyl, cycloalkyl, C₁-C₁₂aryl, substituted alkyl, substituted cycloalkyl and substituted C₁-C₁₂aryl, and
R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from hydrogen, —C(O)NR⁹R¹⁰, —(CH₂)ₙNR⁹R¹⁰, —SO₂NR⁹R¹⁰, —(CH₂)ₙOR⁸, —O—(CH₂)ₘNR⁹R¹⁰ and —N—(CH₂)ₘNR⁹R¹⁰,
where n is 0-2,
m is 1-6, where the carbon chain formed by m is optionally substituted,
R⁸ is alkyl, cycloalkyl, cycloalkyl containing from 1 to 4 heteroatoms, and aryl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 4 heteroatoms, substituted cycloalkyl containing from 1 to 4 heteroatoms, substituted cycloalkyl, halogen, aryl, substituted aryl and protected —OH,
where R² and R³ are independently hydrogen, alkyl, cycloalkyl, C₁-C₁₂aryl, substituted alkyl, substituted cycloalkyl and substituted C₁-C₁₂aryl, and n is 0-2,
R⁹ and R¹⁰ are independently hydrogen, cycloalkyl, cycloalkyl containing from 1 to 4 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 4 heteroatoms, substituted cycloalkyl, halogen, aryl, substituted aryl and protected —OH,
or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine.

4. A pharmaceutically acceptable salt, hydrate, solvate or pro-drug of a compound of Formula (II), as described in claim 3.

5. A compound of claim 1 represented by the following Formula (III): 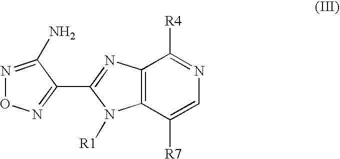
wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶, —S(O)ₙR² and protected —OH,
where n is 0-2,
R² is selected from hydrogen, alkyl, cycloalkyl, C₁-C₁₂aryl, substituted alkyl, substituted cycloalkyl and substituted C₁-C₁₂aryl, and
R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from —C(O)NR⁹R¹⁰, —(CH₂)ₙNR⁹R¹⁰, —SO₂NR⁹R¹⁰, —(CH₂)ₙOR⁸, —O—(CH₂)ₘNR⁹R¹⁰ and —N—(CH₂)ₘNR⁹R¹⁰,
where n is 0-2,
m is 1-6, where the carbon chain formed by m is optionally substituted,
R⁸ is alkyl, piperidine, imidazolidine, phenyl, piperazine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 3 heteroatoms, substituted cycloalkyl containing from 1 to 3 heteroatoms, substituted cycloalkyl, halogen, aryl, substituted aryl and protected —OH,
where R² and R³ are independently hydrogen, alkyl, cycloalkyl, C₁-C₁₂aryl, substituted alkyl, substituted cycloalkyl and substituted C₁-C₁₂aryl, and n is 0-2,
R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl, substituted aryl and protected —OH,
or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine.

6. A pharmaceutically acceptable salt, hydrate, solvate or pro-drug of a compound of Formula (III), as described in claim 5.

7. A compound represented by Formula (II), as defined in claim 3, wherein:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from, —C(O)NR⁹R¹⁰, —(CH₂)ₙNR⁹R¹⁰, —(CH₂)ₙOR⁸, —O—(CH₂)ₘNR⁹R¹⁰ and —N—(CH₂)ₘNR⁹R¹⁰,
where n is 0-2;
m is 1-6, where the carbon chain formed by m is optionally substituted,
R⁸ is alkyl, piperidine, imidazolidine, phenyl, piperazine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, hydroxy, nitro, guanadine, substituted guanadine, cyano, cycloalkyl, substituted cycloalkyl, cycloalkyl containing from 1 to 3 heteroatoms, substituted cycloalkyl containing from 1 to 3 heteroatoms, halogen, C₁-C₁₂aryl and substituted C₁-C₁₂aryl,
R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,
or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl;
except 4-[1-Ethyl-7-(piperidin-4-yloxy)-1H-imidazo[4,5-c]pyridin-2-yl]-furazan-3-ylamine.

8. A pharmaceutically acceptable salt, hydrate, solvate or pro-drug of a compound of Formula (II), as described in claim 7.

9. A compound represented by Formula (III), as defined in claim 5, wherein:
R¹ is selected from: alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro, and halogen; and
R⁷ is selected from —(CH₂)ₙOR⁸, —O—(CH₂)ₘNR⁹R¹⁰ and —N—(CH₂)ₘNR⁹R¹⁰,
where n is 0-2;
m is 1-6, where the carbon chain formed by m is optionally substituted,
R⁸ is alkyl, piperidine, imidazolidine, phenyl, piperazine, piperidyl and pyrrolidinyl, each of which is optionally substituted with one or more substituents selected from the group consisting of: alkoxy, acyloxy, aryloxy, amino, amino substituted with one or more substituents selected from the group consisting of: hydroxy, alkoxy and amino, N-acylamino, hydroxy, nitro, guanadine, substituted guanadine, cyano, cycloalkyl, substituted cycloalkyl, cycloalkyl containing from 1 to 3 heteroatoms, substituted cycloalkyl containing from 1 to 3 heteroatoms, halogen, C₁-C₁₂aryl and substituted C₁-C₁₂aryl,
R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,
or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl;
except 4-[1-Ethyl-7-(piperidin-4-yloxy)-1H-imidazo[4,5-c]pyridin-2-yl]-furazan-3-ylamine.

10. A pharmaceutically acceptable salt, hydrate, solvate or pro-drug of a compound of Formula (II), as described in claim 9.

11. A compound represented by Formula (I), as defined in claim 1, wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶ and —S(O)ₙR²,
where n is 0-2,
R² is selected from hydrogen, alkyl, cycloalkyl, C₁-C₁₂aryl, substituted alkyl, substituted cycloalkyl and substituted C₁-C₁₂aryl, and
R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of:
alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl and substituted aryl, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from —(CH₂)ₙOR⁸, —O—(CH₂)ₘNR⁹R¹⁰ and —N—(CH₂)ₘNR⁹R¹⁰,
where n is 0-2,
m is 1-6, where the carbon chain formed by m is optionally substituted,
R⁸ is alkyl substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 3 heteroatoms, substituted cycloalkyl, substituted cycloalkyl containing from 1 to 3 heteroatoms, halogen, aryl and substituted aryl, cycloalkyl and cycloalkyl containing from 1 to 3 heteroatoms, each of said cycloalkyl and cycloalkyl containing from 1 to 3 heteroatoms is optionally substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, cycloalkyl containing from 1 to 3 heteroatoms, substituted cycloalkyl, halogen, aryl and substituted aryl,
where R² and R³ are independently hydrogen, alkyl, cycloalkyl, C₁-C₁₂aryl, substituted alkyl, substituted cycloalkyl and substituted C₁-C₁₂aryl, and n is 0-2,
R⁹ and R¹⁰ are hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine.

12. A pharmaceutically acceptable salt, hydrate, solvate or pro-drug of a compound of Formula (I), as described in claim 11.

13. A compound represented by Formula (II), as defined in claim 3, wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, alkoxy, acetamide, cyano, nitrile, urea, substituted urea, aryl, substituted cycloalkyl, substituted aryl, aryloxy, oxo, hydroxy, alkoxy, cycloalkyl, acyloxy, amino, N-acylamino, nitro, halogen, —C(O)OR², —C(O)NR⁵R⁶, —S(O)₂NR⁵R⁶ and —S(O)ₙR²,
where n is 0-2,
R² is selected from hydrogen, alkyl, cycloalkyl, C₁-C₁₂aryl, substituted alkyl, substituted cycloalkyl and substituted C₁-C₁₂aryl, and
R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl and substituted aryl, or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from —(CH₂)ₙOR⁸, —O—(CH₂)ₘNR⁹R¹⁰ and —N—(CH₂)ₘNR⁹R¹⁰,
where n is 0-2,
m is 1-6, where the carbon chain formed by m is optionally substituted,
R⁸ is alkyl substituted with one or more substituents selected from the group consisting of: alkoxy, acyloxy, aryloxy, amino, amino substituted with one or more substituents selected from the group consisting of: hydroxy, alkoxy and amino, N-acylamino, oxo, hydroxy, —C(O)OR², —S(O)ₙR², —C(O)NR²R³, —S(O)₂NR²R³, nitro, guanadine, substituted guanadine, cyano, cycloalkyl, cycloalkyl containing from 1 to 3 heteroatoms, substituted cycloalkyl, substituted cycloalkyl containing from 1 to 3 heteroatoms, halogen, aryl and substituted aryl, cycloalkyl and cycloalkyl containing from 1 to 3 heteroatoms, each of said cycloalkyl and cycloalkyl containing from 1 to 3 heteroatoms is optionally substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, cycloalkyl containing from 1 to 3 heteroatoms, substituted cycloalkyl, halogen, aryl and substituted aryl,
where R² and R³ are independently hydrogen, alkyl, cycloalkyl, C₁-C₁₂aryl, substituted alkyl, substituted cycloalkyl and substituted C₁-C₁₂aryl, and n is 0-2,
R⁹ and R¹⁰ are hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
where R² and R³ are independently hydrogen, alkyl, cycloalkyl, C₁-C₁₂aryl, substituted alkyl, substituted cycloalkyl and substituted C₁-C₁₂aryl, and n is 0-2;
except 4-[1-Ethyl-7-(piperidin-4-yloxy)-1H-imidazo[4,5-c]pyridin-2-yl]-furazan-3-ylamine.

14. A pharmaceutically acceptable salt, hydrate, solvate or pro-drug of a compound of Formula (II), as described in claim 13.

15. A compound represented by Formula (I), as defined in claim 1, wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
where n is 0-2,
m is 1-6, where the carbon chain formed by m is optionally substituted,
R⁸ is alkyl substituted with one or more substituents selected from the group consisting of: cycloalkyl, cycloalkyl containing from 1 to 3 heteroatoms, substituted cycloalkyl, substituted cycloalkyl containing from 1 to 3 heteroatoms, aryl and substituted aryl,
R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
where R² and R³ are independently hydrogen, alkyl, cycloalkyl, C₁-C₁₂aryl, substituted alkyl, substituted cycloalkyl and substituted C₁-C₁₂aryl, and n is 0-2.

16. A pharmaceutically acceptable salt, hydrate, solvate or pro-drug of a compound of Formula (I), as described in claim 15.

17. A compound represented by Formula (II), as defined in claim 3, wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
where n is 0-2,
m is 1-6, where the carbon chain formed by m is optionally substituted,
R⁸ is alkyl substituted with one or more substituents selected from the group consisting of: cycloalkyl, cycloalkyl containing from 1 to 3 heteroatoms, substituted cycloalkyl, substituted cycloalkyl containing from 1 to 3 heteroatoms, aryl and substituted aryl,
R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, cycloalkyl containing from 1 to 3 heteroatoms, substituted cycloalkyl, halogen, aryl and substituted aryl,
where R² and R³ are independently hydrogen, alkyl, cycloalkyl, C₁-C₁₂aryl, substituted alkyl, substituted cycloalkyl and substituted C₁-C₁₂aryl, and n is 0-2.

18. A pharmaceutically acceptable salt, hydrate, solvate or pro-drug of a compound of Formula (II), as described in claim 17.

19. A compound represented by Formula (I), as defined in claim 1, wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
where n is 0-2,
m is 1-6, where the carbon chain formed by m is optionally substituted,
R⁸ is alkyl substituted with one or more substituents selected from the group consisting of: piperidine, substituted piperidine, phenyl and, substituted phenyl,
R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, nitro, cyano, cycloalkyl, cycloalkyl containing from 1 to 3 heteroatoms, halogen and aryl.

20. A pharmaceutically acceptable salt, hydrate, solvate or pro-drug of a compound of Formula (I), as described in claim 19.

21. A compound represented by Formula (II), as defined in claim 3, wherein:
R¹ is selected from alkyl, alkyl substituted with one or more substituents selected from the group consisting of: hydroxy, alkoxy, amino, N-acylamino, cyclopropyl and halogen, cycloalkyl, cycloalkyl containing from 1 to 3 heteroatoms and C₁-C₁₂aryl;
R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, alkoxy, acetamide, cyano, nitrile, urea, substituted urea, aryloxy, hydroxy, alkoxy, acyloxy, amino, N-acylamino, nitro and halogen; and
R⁷ is selected from —(CH₂)ₙOR⁸, —O—(CH₂)ₘNR⁸R⁹ and —N—(CH₂)ₘNR⁸R⁹,
where n is 0-2,
m is 1-6, where the carbon chain formed by m is optionally substituted,
R⁸ is alkyl substituted with one or more substituents selected from the group consisting of: piperidine, substituted piperidine, phenyl and, substituted phenyl,
R⁹ is hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, nitro, cyano, cycloalkyl, cycloalkyl containing from 1 to 3 heteroatoms, halogen and aryl.

22. A pharmaceutically acceptable salt, hydrate, solvate or pro-drug of a compound of Formula (II), as described in claim 21.

23. A compound of claim 1 selected from:
4-(4-Phenyl-1-piperidin-4-yl-1H-imidazo[4,5-c]pyridin-2-yl)-furazan-3-ylamine;
4-[4-(3-Chloro-phenyl)-1-piperidin-4-yl-1H-imidazo-[4,5-c]pyridin-2-yl]furazan-3-ylamine;
4-[1-(3-Amino-2,2-dimethylpropyl)-4-(3-chlorophenyl)-1H-imidazo[4,5-c]pyridin-2-yl]-furazan-3-ylamine;
4-[1-(cyclopropylmethyl)-4-(2-methylphenyl)-1H-imidazo[4,5-c]pyridin-2-yl]-1,2,5-oxadiazol-3-amine;
4-[4-(2-chlorophenyl)-1-(cyclopropylmethyl)-1H-imidazo[4,5-c]pyridin-2-yl]-1,2,5-oxadiazol-3-amine;
4-[1-(3-Amino-2,2-dimethylpropyl)-4-phenyl-1H-imidazo[4,5-c]pyridinyl-2-yl]-furazan-3-ylamine;
4-[4-(3-chlorophenyl)-1-(cyclopropylmethyl)-1H-imidazo[4,5-c]pyridin-2-yl]-1,2,5-oxadiazol-3-amine;
4-[4-chloro-1-(cyclopropylmethyl)-1H-imidazo[4,5-c]pyridin-2-yl]-1,2,5-oxadiazol-3-amine;
4-[1-(cyclopropylmethyl)-4-(3-furanyl)-1H-imidazo[4,5-c]pyridin-2-yl]-1,2,5-oxadiazol-3-amine;
4-[1-(5-aminopentyl)-4-phenyl-1H-imidazo[4,5-c]pyridin-2-yl]-1,2,5-oxadiazol-3-amine;
4-[1-(6-aminohexyl)-4-phenyl-1H-imidazo[4,5-c]pyridin-2-yl]-1,2,5-oxadiazol-3-amine;
4-[1-(5-aminopentyl)-4-(3-chlorophenyl)-1H-imidazo[4,5-c]pyridin-2-yl]-1,2,5-oxadiazol-3-amine;
4-[1-(6-aminohexyl)-4-(3-chlorophenyl)-1H-imidazo[4,5-c]pyridin-2-yl]-1,2,5-oxadiazol-3-amine;
4-[1-(3-Amino-2,2-dimethylpropyl)-4-(3-methoxyphenyl)-1H-imidazo[4,5-c]pyridinyl-2-yl]-furazan-3-ylamine;
4-[1-(5-aminopentyl)-4-(3-thienyl)-1H-imidazo[4,5-c]pyridin-2-yl]-1,2,5-oxadiazol-3-amine;
4-[1-(6-aminohexyl)-4-(3-thienyl)-1H-imidazo[4,5-c]pyridin-2-yl]-1,2,5-oxadiazol-3-amine;
4-[4-phenyl-1-(3-piperidinylmethyl)-1H-imidazo[4,5-c]pyridin-2-yl]-1,2,5-oxadiazol-3-amine;
4-[4-(3-chlorophenyl)-1-(3-piperidinylmethyl)-1H-imidazo[4,5-c]pyridin-2-yl]-1,2,5-oxadiazol-3-amine;
4-[4-(4-chlorophenyl)-1-(3-piperidinylmethyl)-1H-imidazo[4,5-c]pyridin-2-yl]-1,2,5-oxadiazol-3-amine;
4-[1-(3-aminopropyl)-4-(2-thienyl)-1H-imidazo[4,5-c]pyridin-2-yl]-1,2,5-oxadiazol-3-amine;
4-[1-(3-aminopropyl)-4-(1-piperidinyl)-1H-imidazo[4,5-c]pyridin-2-yl]-1,2,5-oxadiazol-3-amine;
1-[2-(4-Aminofurazan-3-yl)-1-ethyl-4-phenyl-1-H-imidazo[4,5-c]pyridin-7-yl]-1-(3-aminopyrrolidin-1-yl)methanone;
1-[2-(4-Aminofurazan-3-yl)-1-ethyl-4-thiophen-3-yl-1-H-imidazo[4,5-c]pyridin-7-yl]-1-(3-aminopyrrolidin-1-yl)methanone;
1-[2-(4-Aminofurazan-3-yl)-1-ethyl-4-pyridin-yl-1-H-imidazo[4,5-c]pyridin-7-yl]-1-(3-aminopyrrolidin-1-yl)methanone;
1-[2-(4-Aminofurazan-3-yl)-1-ethyl-4-pyridin-3-yl-1-H-imidazo[4,5-c]pyridin-7-yl]-1-(3-aminopyrrolidin-1-yl)methanone;
1-[2-(4-Aminofurazan-3-yl)-1-ethyl-4-furan-3-yl-1-H-imidazo[4,5-c]pyridin-7-yl]-1-(3-aminopyrrolidin-1-yl)methanone;
1-[2-(4-Amino-furazan-3-yl)-4-chloro-1-ethyl-1-H-imidazo[4,5-c]pyridin-7-yl]-1-(3-amino-pyrrolidin-1-yl)-methanone;
1-[2-(4-Amino-furazan-3-yl)-4-(1H-pyrrol-2-yl))-1-ethyl-1-H-imidazo[4,5-c]pyridin-7-yl]-1-(3-amino-pyrrolidin-1-yl)-methanone;
1-[2-(4-Amino-furazan-3-yl)-1-ethyl-4-(2-methoxyphenyl)-1H-imidazo[4,5-c]pyridin-7-yl]-1-(3-amino-pyrrolidin-1-yl)-methanone;
1-[2-(4-Amino-furazan-3-yl)-1-ethyl-4-(3-chloro-phenyl)-1H-imidazo[4,5-c]pyridin-7-yl]-1-(3-amino-pyrrolidin-1-yl)-methanone;
1-[2-(4-Amino-furazan-3-yl)-1-ethyl-4-furan-2-yl-1H-imidazo[4,5-c]pyridin-7-yl]-1-(3-amino-pyrrolidin-1-yl)-methanone;
2-(4-Amino-furazan-3-yl)-1-ethyl-4-phenyl-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(3-chloro-phenyl)-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(2,3-dichloro-phenyl)-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(2-chloro-phenyl)-1H-imidazo[4,5-c]pyridine-7-carboxylic acid [1-(4-chloro-benzyl)-2-hydroxy-ethyl]-amide;
2-(4-Amino-furazan-3-yl)-1-ethyl-4-(2-hydroxy-phenyl)-1H-imidazo[4,5-c]pyridine-7-carboxylic acid [1-(4-chloro-benzyl)-2-hydroxy-ethyl]-amide,
2-(4-Amino-furazan-3-yl)-4-(3-chloro-phenyl)-1-ethyl-1H-imidazo[4,5-c]pyridine-7-carboxylic acid pyrrolidin-3-ylamide;
2-(4-Amino-furazan-3-yl)-4-phenyl-1-ethyl-1H-imidazo[4,5-c]pyridine-7-carboxylic acid pyrrolidin-3-ylamide;
2-(4-Amino-furazan-3-yl)-4-(5-chloro-thiophen-2-yl)-1-ethyl-1H-imidazo[4,5-c]pyridine-7-carboxylic acid pyrrolidin-3-ylamide;
2-(4-Amino-furazan-3-yl)-4-(2-amino-phenyl)-1-ethyl-1H-imidazo[4,5-c]pyridine-7-carboxylic acid pyrrolidin-3-ylamide;
2-(4-Amino-furazan-3-yl)-4-(3-amino-phenyl)-1-ethyl-1H-imidazo[4,5-c]pyridine-7-carboxylic acid pyrrolidin-3-ylamide;
2-(4-Amino-furazan-3-yl)-4-(3-bromo-phenyl)-1-ethyl-1H-imidazo[4,5-c]pyridine-7-carboxylic acid pyrrolidin-3-ylamide;
2-(4-Amino-furazan-3-yl)-4-(1-naphthalenyl)-1-ethyl-1H-imidazo[4,5-c]pyridine-7-carboxylic acid pyrrolidin-3-ylamide;
2-(4-Amino-furazan-3-yl)-4-(thiophen-2-yl)-1-ethyl-1H-imidazo[4,5-c]pyridine-7-carboxylic acid pyrrolidin-3-ylamide;
2-(4-Amino-furazan-3-yl)-4-(3,4-methylenedioxyphenyl)-1-ethyl-1H-imidazo[4,5-c]pyridine-7-carboxylic acid pyrrolidin-3-ylamide;
2-(4-Amino-furazan-3-yl)-4-(3,5-dichloro-phenyl)-1-ethyl-1H-imidazo[4,5-c]pyridine-7-carboxylic acid pyrrolidin-3-ylamide;
4-[7-[(3-amino-1-pyrrolidinyl)carbonyl]-4-(3-chlorophenyl)-1-(cyclopropylmethyl)-1H-imidazo[4,5-c]pyridin-2-yl]-1,2,5-oxadiazol-3-amine;
4-[7-[(3-amino-1-pyrrolidinyl)carbonyl]-4-(4-biphenylyl)-1-ethyl-1H-imidazo[4,5-c]pyridin-2-yl]-1,2,5-oxadiazol-3-amine;
4-[7-[(3-amino-1-pyrrolidinyl)carbonyl]-4-(2,4-dichlorophenyl)-1-ethyl-1H-imidazo[4,5-c]pyridin-2-yl]-1,2,5-oxadiazol-3-amine;
4-[7-[(3-amino-1-pyrrolidinyl)carbonyl]-1-ethyl-4-(phenylethynyl)-1H-imidazo[4,5-c]pyridin-2-yl]-1,2,5-oxadiazol-3-amine;
2-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol;
4-[7-[(3-amino-1-pyrrolidinyl)carbonyl]-4-(2-chlorophenyl)-1-ethyl-1H-imidazo[4,5-c]pyridin-2-yl]-1,2,5-oxadiazol-3-amine;
(2-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenyl)methanol;
2-{2-(4-amino-1,2,5-oxadiazol-3-yl)-7-[(3-amino-1-pyrrolidinyl)carbonyl]-1-ethyl-1H-imidazo[4,5-c]pyridin-4-yl}-4-chlorophenol;
4-(1-ethyl-7-{[3-(methylamino)-1-pyrrolidinyl]carbonyl}-4-phenyl-1H-imidazo[4,5-c]pyridin-2-yl)-1,2,5-oxadiazol-3-amine;
4-[7-[(3-amino-1-pyrrolidinyl)carbonyl]-1-ethyl-4-(4-methylphenyl)-1H-imidazo[4,5-c]pyridin-2-yl]-1,2,5-oxadiazol-3-amine;
4-[7-[(3-amino-1-pyrrolidinyl)carbonyl]-4-(2,5-dichlorophenyl)-1-ethyl-1H-imidazo[4,5-c]pyridin-2-yl]-1,2,5-oxadiazol-3-amine;
4-[7-[(3-amino-1-pyrrolidinyl)carbonyl]-4-(1-benzothien-2-yl)-1-ethyl-1H-imidazo[4,5-c]pyridin-2-yl]-1,2,5-oxadiazol-3-amine;
4-[1-ethyl-4-phenyl-7-(4-piperidinyloxy)-1H-imidazo[4,5-c]pyridin-2-yl]-1,2,5-oxadiazol-3-amine;
4-{7-[(3-amino-1-pyrrolidinyl)carbonyl]-1-ethyl-4-[4-(methyloxy)phenyl]-1H-imidazo[4,5-c]pyridin-2-yl}-1,2,5-oxadiazol-3-amine;
4-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol;
4-[7-[(3-amino-1-pyrrolidinyl)carbonyl]-4-(4-chlorophenyl)-1-ethyl-1H-imidazo[4,5-c]pyridin-2-yl]-1,2,5-oxadiazol-3-amine;
4-[4-(3-chlorophenyl)-1-ethyl-7-(4-piperidinyloxy)-1H-imidazo[4,5-c]pyridin-2-yl]-1,2,5-oxadiazol-3-amine;
2-(4-amino-1,2,5-oxadiazol-3-yl)-4-(3-chlorophenyl)-1-(cyclopropylmethyl)-N-{2-[(phenylmethyl)amino]ethyl}-1H-imidazo[4,5-c]pyridine-7-carboxamide;
3-{2-(4-amino-1,2,5-oxadiazol-3-yl)-7-[(3-amino-1-pyrrolidinyl)carbonyl]-1-ethyl-1H-imidazo[4,5-c]pyridin-4-yl}phenol;
4-{2-(4-amino-1,2,5-oxadiazol-3-yl)-7-[(3-amino-1-pyrrolidinyl)carbonyl]-1-ethyl-1H-imidazo[4,5-c]pyridin-4-yl}benzonitrile;
1-[2-(4-Amino-furazan-3-yl)-4-phenyl-1-piperidin-4yl-1-H-imidazo[4,5-c]pyridin-7-yl]-1-(3-amino-pyrrolidin-1-yl)-methanone;
4-(4-(3-chlorophenyl)-1-ethyl-7-{[3-(methylamino)-1-pyrrolidinyl]carbonyl}-1H-imidazo[4,5-c]pyridin-2-yl)-1,2,5-oxadiazol-3-amine;
4-(4-(2,5-dichlorophenyl)-1-ethyl-7-{[3-(methylamino)-1-pyrrolidinyl]carbonyl}-1H-imidazo[4,5-c]pyridin-2-yl)-1,2,5-oxadiazol-3-amine;
4-[4-(2,5-dichlorophenyl)-1-ethyl-7-(4-piperidinyloxy)-1H-imidazo[4,5-c]pyridin-2-yl]-1,2,5-oxadiazol-3-amine;
2-(4-amino-1,2,5-oxadiazol-3-yl)-4-(3-chlorophenyl)-1-(cyclopropylmethyl)-N-[3-(dimethylamino)propyl]-1H-imidazo[4,5-c]pyridine-7-carboxamide;
4-[7-[(3-amino-1-pyrrolidinyl)carbonyl]-1-ethyl-4-(1H-pyrrol-2-yl)-1H-imidazo[4,5-c]pyridin-2-yl]-1,2,5-oxadiazol-3-amine;
4-[7-[(3-amino-1-pyrrolidinyl)carbonyl]-4-(4-bromophenyl)-1-ethyl-1H-imidazo[4,5-c]pyridin-2-yl]-1,2,5-oxadiazol-3-amine;
4-[7-[(3-amino-1-pyrrolidinyl)carbonyl]-4-phenyl-1-(4-piperidinyl)-1H-imidazo[4,5-c]pyridin-2-yl]-1,2,5-oxadiazol-3-amine;
4-{7-[(4-aminobutyl)oxy]-1-ethyl-4-phenyl-1H-imidazo[4,5-c]pyridin-2-yl}-1,2,5-oxadiazol-3-amine;
4-{1-ethyl-4-phenyl-7-[(4-piperidinylmethyl)oxy]-1H-imidazo[4,5-c]pyridin-2-yl}-1,2,5-oxadiazol-3-amine;
4-{4-(3-chlorophenyl)-1-ethyl-7-[(4-piperidinylmethyl)oxy]-1H-imidazo[4,5-c]pyridin-2-yl}-1,2,5-oxadiazol-3-amine;
4-[7-[(4-aminobutyl)oxy]-4-(3-chlorophenyl)-1-ethyl-1H-imidazo[4,5-c]pyridin-2-yl]-1,2,5-oxadiazol-3-amine;
4-{7-[(2-aminoethyl)oxy]-1-ethyl-4-phenyl-1H-imidazo[4,5-c]pyridin-2-yl}-1,2,5-oxadiazol-3-amine;
4-{1-ethyl-4-phenyl-7-[(3-pyrrolidinylmethyl)oxy]-1H-imidazo[4,5-c]pyridin-2-yl}-1,2,5-oxadiazol-3-amine;
4-{7-[(3-aminopropyl)oxy]-1-ethyl-4-phenyl-1H-imidazo[4,5-c]pyridin-2-yl}-1,2,5-oxadiazol-3-amine;
4-(7-{[(2S)-2-amino-3-phenylpropyl]oxy}-1-ethyl-4-phenyl-1H-imidazo[4,5-c]pyridin-2-yl)-1,2,5-oxadiazol-3-amine;
4-[1-ethyl-4-phenyl-7-(3-piperidinyloxy)-1H-imidazo[4,5-c]pyridin-2-yl]-1,2,5-oxadiazol-3-amine;
2-(4-amino-1,2,5-oxadiazol-3-yl)-1-ethyl-N-methyl-N-(1-methyl-4-piperidinyl)-4-phenyl-1H-imidazo[4,5-c]pyridine-7-carboxamide;
N-{[2-(4-amino-1,2,5-oxadiazol-3-yl)-1-ethyl-4-phenyl-1H-imidazo[4,5-c]pyridin-7-yl]methyl}-N,1-dimethyl-4-piperidinamine;
4-(1-ethyl-4-phenyl-7-{[2-(4-piperidinyl)ethyl]oxy}-1H-imidazo[4,5-c]pyridin-2-yl)-1,2,5-oxadiazol-3-amine;
4-{1-(4-aminobutyl)-7-[(3-amino-1-pyrrolidinyl)carbonyl]-4-phenyl-1H-imidazo[4,5-c]pyridin-2-yl}-1,2,5-oxadiazol-3-amine;
4-(7-{[(2R)-2-amino-3-phenylpropyl]oxy)-1-ethyl-4-phenyl-1H-imidazo[4,5-c]pyridin-2-yl)-1,2,5-oxadiazol-3-amine;
4-{1-(4-aminobutyl)-7-[(3-amino-1-pyrrolidinyl)carbonyl]-4-phenyl-1H-imidazo[4,5-c]pyridin-2-yl}-1,2,5-oxadiazol-3-amine;
4-(1-(4-aminobutyl)-7-{[3-(methylamino)-1-pyrrolidinyl]carbonyl}-4-phenyl-1H-imidazo[4,5-c]pyridin-2-yl)-1,2,5-oxadiazol-3-amine;
4-{1-ethyl-7-[(4-methyl-1-piperazinyl)methyl]-4-phenyl-1H-imidazo[4,5-c]pyridin-2-yl}-1,2,5-oxadiazol-3-amine;
4-(1-ethyl-7-{[3-(methylamino)-1-pyrrolidinyl]methyl}-4-phenyl-1H-imidazo[4,5-c]pyridin-2-yl)-1,2,5-oxadiazol-3-amine;
(3-amino-2,2-dimethylpropyl){[2-(4-amino-1,2,5-oxadiazol-3-yl)-1-ethyl-4-phenyl-1H-imidazo[4,5-c]pyridin-7-yl]methyl}amine;
4-(7-{[3-(dimethylamino)-1-pyrrolidinyl]methyl}-1-ethyl-4-phenyl-1H-imidazo[4,5-c]pyridin-2-yl)-1,2,5-oxadiazol-3-amine;
4-(1-ethyl-7-{[2-(methylamino)ethyl]oxy}-4-phenyl-1H-imidazo[4,5-c]pyridin-2-yl)-1,2,5-oxadiazol-3-amine;
4-[1-ethyl-4-phenyl-7-({2-[(phenylmethyl)amino]ethyl}oxy)-1H-imidazo[4,5-c]pyridin-2-yl]-1,2,5-oxadiazol-3-amine;
4-{1-ethyl-4-phenyl-7-[(3-piperidinylmethyl)oxy]-1H-imidazo[4,5-c]pyridin-2-yl}-1,2,5-oxadiazol-3-amine;
4-{7-[(5-aminopentyl)oxy]-1-ethyl-4-phenyl-1H-imidazo[4,5-c]pyridin-2-yl}-1,2,5-oxadiazol-3-amine;
4-(7-{[3-(dimethylamino)-2,2-dimethylpropyl]oxy}-1-ethyl-4-phenyl-1H-imidazo[4,5-c]pyridin-2-yl)-1,2,5-oxadiazol-3-amine;
1-(4-aminobutyl)-2-(4-amino-1,2,5-oxadiazol-3-yl)-4-phenyl-N-{2-[(phenylmethyl)amino]ethyl}-1H-imidazo[4,5-c]pyridine-7-carboxamide;
2-(4-amino-1,2,5-oxadiazol-3-yl)-1-(1-methylethyl)-4-phenyl-N-3-pyrrolidinyl-1H-imidazo[4,5-c]pyridine-7-carboxamide;
4-[7-{[3-(methylamino)-1-pyrrolidinyl]carbonyl}-1-(1-methylethyl)-4-phenyl-1H-imidazo[4,5-c]pyridin-2-yl]-1,2,5-oxadiazol-3-amine;
4-(7-{[(3S)-3-amino-1-pyrrolidinyl]methyl}-1-ethyl-4-phenyl-1H-imidazo[4,5-c]pyridin-2-yl)-1,2,5-oxadiazol-3-amine;
4-[1-ethyl-7-(hexahydro-1H-1,4-diazepin-1-ylmethyl)-4-phenyl-1H-imidazo[4,5-c]pyridin-2-yl]-1,2,5-oxadiazol-3-amine;
4-[1-ethyl-4-phenyl-7-(1-piperazinylmethyl)-1H-imidazo[4,5-c]pyridin-2-yl]-1,2,5-oxadiazol-3-amine;
4-(7-{[2-(dimethylamino)ethyl]oxy}-1-ethyl-4-phenyl-1H-imidazo[4,5-c]pyridin-2-yl)-1,2,5-oxadiazol-3-amine;
4-(1-ethyl-4-phenyl-7-{[(2S)-2-pyrrolidinylmethyl]oxy}-1H-imidazo[4,5-c]pyridin-2-yl)-1,2,5-oxadiazol-3-amine;
4-(1-ethyl-4-phenyl-7-{[(2R)-2-pyrrolidinylmethyl]oxy}-1H-imidazo[4,5-c]pyridin-2-yl)-1,2,5-oxadiazol-3-amine;
2-(4-amino-1,2,5-oxadiazol-3-yl)-N-(3-aminopropyl)-1-(1-methylethyl)-4-phenyl-1H-imidazo[4,5-c]pyridine-7-carboxamide;
2-(4-amino-1,2,5-oxadiazol-3-yl)-1-(1-methylethyl)-4-phenyl-N²-propen-1-yl-1H-imidazo[4,5-c]pyridine-7-carboxamide;
2-(4-amino-1,2,5-oxadiazol-3-yl)-1-ethyl-N-[3-(4-morpholinyl)propyl]-4-phenyl-1H-imidazo[4,5-c]pyridine-7-carboxamide;
2-(4-amino-1,2,5-oxadiazol-3-yl)-1-ethyl-N-[2-(1H-imidazol-4-yl)ethyl]-4-phenyl-1H-imidazo[4,5-c]pyridine-7-carboxamide;
2-(4-amino-1,2,5-oxadiazol-3-yl)-1-ethyl-N-[3-(4-methyl-1-piperazinyl)propyl]-4-phenyl-1H-imidazo[4,5-c]pyridine-7-carboxamide;
4-[7-[(3-aminopropyl)oxy]-4-(2-chlorophenyl)-1-ethyl-1H-imidazo[4,5-c]pyridin-2-yl]-1,2,5-oxadiazol-3-amine;
4-[7-[(3-aminopropyl)oxy]-4-(3-chlorophenyl)-1-ethyl-1H-imidazo[4,5-c]pyridin-2-yl]-1,2,5-oxadiazol-3-amine;
4-[7-[(3-aminopropyl)oxy]-4-(4-chlorophenyl)-1-ethyl-1H-imidazo[4,5-c]pyridin-2-yl]-1,2,5-oxadiazol-3-amine;
4-{7-[(3-aminopropyl)oxy]-4-[5-chloro-2-(methyloxy)phenyl]-1-ethyl-1H-imidazo[4,5-c]pyridin-2-yl}-1,2,5-oxadiazol-3-amine;
N-(1-{[2-(4-amino-1,2,5-oxadiazol-3-yl)-1-ethyl-4-phenyl-1H-imidazo[4,5-c]pyridin-7-yl]carbonyl}-3-pyrrolidinyl)-N-methylacetamide;
2-(4-amino-1,2,5-oxadiazol-3-yl)-N-[3-(dimethylamino)propyl]-1-ethyl-4-phenyl-1H-imidazo[4,5-c]pyridine-7-carboxamide;
2-{2-(4-amino-1,2,5-oxadiazol-3-yl)-7-[(3-aminopropyl)oxy]-1-ethyl-1H-imidazo[4,5-c]pyridin-4-yl}-4-chlorophenol;
4-[7-[(3-aminopropyl)oxy]-1-ethyl-4-(2-pyridinyl)-1H-imidazo[4,5-c]pyridin-2-yl]-1,2,5-oxadiazol-3-amine;
4-(7-{[3-(dimethylamino)propyl]oxy}-1-ethyl-4-phenyl-1H-imidazo[4,5-c]pyridin-2-yl)-1,2,5-oxadiazol-3-amine;
4-(1-ethyl-7-{[3-(4-morpholinyl)propyl]oxy}-4-phenyl-1H-imidazo[4,5-c]pyridin-2-yl)-1,2,5-oxadiazol-3-amine;
2-(4-amino-1,2,5-oxadiazol-3-yl)-1-cyclopentyl-4-phenyl-N-3-pyrrolidinyl-1H-imidazo[4,5-c]pyridine-7-carboxamide;
4-{7-[(3-amino-1-pyrrolidinyl)carbonyl]-1-cyclopentyl-4-phenyl-1H-imidazo[4,5-c]pyridin-2-yl}-1,2,5-oxadiazol-3-amine;
4-(1-cyclopentyl-7-{[3-(methylamino)-1-pyrrolidinyl]carbonyl}-4-phenyl-1H-imidazo[4,5-c]pyridin-2-yl)-1,2,5-oxadiazol-3-amine;
4-(1-ethyl-7-{[3-(methylamino)propyl]oxy}-4-phenyl-1H-imidazo[4,5-c]pyridin-2-yl)-1,2,5-oxadiazol-3-amine;
4-{1-ethyl-7-[(3-hydrazinopropyl)oxy]-4-phenyl-1H-imidazo[4,5-c]pyridin-2-yl}-1,2,5-oxadiazol-3-amine;
2-[(3-{[2-(4-amino-1,2,5-oxadiazol-3-yl)-1-ethyl-4-phenyl-1H-imidazo[4,5-c]pyridin-7-yl]oxy}propyl)amino]ethanol;
4-(1-ethyl-7-{[3-(hydroxyamino)propyl]oxy}-4-phenyl-1H-imidazo[4,5-c]pyridin-2-yl)-1,2,5-oxadiazol-3-amine;
(3R)-1-{[2-(4-amino-1,2,5-oxadiazol-3-yl)-1-ethyl-4-phenyl-1H-imidazo[4,5-c]pyridin-7-yl]carbonyl}-3-pyrrolidinol;
2-(4-amino-1,2,5-oxadiazol-3-yl)-N-[3-(diethylamino)propyl]-1-ethyl-4-phenyl-1H-imidazo[4,5-c]pyridine-7-carboxamide;
2-(4-amino-1,2,5-oxadiazol-3-yl)-1-ethyl-N-[3-(2-methyl-1-piperidinyl)propyl]-4-phenyl-1H-imidazo[4,5-c]pyridine-7-carboxamide;
4-(1-methyl-7-{[3-(methylamino)-1-pyrrolidinyl]carbonyl}-4-phenyl-1H-imidazo[4,5-c]pyridin-2-yl)-1,2,5-oxadiazol-3-amine;
4-{7-[(3-amino-1-pyrrolidinyl)carbonyl]-1-methyl-4-phenyl-1H-imidazo[4,5-c]pyridin-2-yl}-1,2,5-oxadiazol-3-amine;
4-(1-butyl-7-{[3-(methylamino)-1-pyrrolidinyl]carbonyl}-4-phenyl-1H-imidazo[4,5-c]pyridin-2-yl)-1,2,5-oxadiazol-3-amine;
4-{7-[(3-amino-1-pyrrolidinyl)carbonyl]-1-butyl-4-phenyl-1H-imidazo[4,5-c]pyridin-2-yl}-1,2,5-oxadiazol-3-amine;
4-[7-[(3-amino-1-pyrrolidinyl)carbonyl]-1-(4-fluorophenyl)-4-phenyl-1H-imidazo[4,5-c]pyridin-2-yl]-1,2,5-oxadiazol-3-amine;
N-(2-aminoethyl)-2-(4-amino-1,2,5-oxadiazol-3-yl)-1-(4-fluorophenyl)-4-phenyl-1H-imidazo[4,5-c]pyridine-7-carboxamide;
4-{1-(4-aminophenyl)-7-[(3-amino-1-pyrrolidinyl)carbonyl]-4-phenyl-1H-imidazo[4,5-c]pyridin-2-yl}-1,2,5-oxadiazol-3-amine;
1-{[2-(4-amino-1,2,5-oxadiazol-3-yl)-1-ethyl-4-phenyl-1H-imidazo[4,5-c]pyridin-7-yl]oxy}-3-(4-morpholinyl)-2-propanol;
N-[2-(4-amino-1,2,5-oxadiazol-3-yl)-1-ethyl-4-phenyl-1H-imidazo[4,5-c]pyridin-7-yl]-4-piperidinecarboxamide;
4-[7-{[3-(dimethylamino)-1-pyrrolidinyl]carbonyl}-4-phenyl-1-(2,2,2-trifluoroethyl)-1H-imidazo[4,5-c]pyridin-2-yl]-1,2,5-oxadiazol-3-amine;
4-(1-ethyl-7-{[2-(4-morpholinyl)ethyl]oxy}-4-phenyl-1H-imidazo[4,5-c]pyridin-2-yl)-1,2,5-oxadiazol-3-amine;
4-(1-ethyl-4-phenyl-7-{[3-(1-piperidinyl)propyl]oxy}-1H-imidazo[4,5-c]pyridin-2-yl)-1,2,5-oxadiazol-3-amine trifluoroacetate;
2-(4-amino-1,2,5-oxadiazol-3-yl)-1-ethyl-N-[2-(1-methyl-2-pyrrolidinyl)ethyl]-4-phenyl-1H-imidazo[4,5-c]pyridine-7-carboxamide;
1-(1-{[2-(4-amino-1,2,5-oxadiazol-3-yl)-1-ethyl-4-phenyl-1H-imidazo[4,5-c]pyridin-7-yl]carbonyl}-4-piperidinyl)-1,3-dihydro-2H-benzimidazol-2-one;
1-{[2-(4-amino-1,2,5-oxadiazol-3-yl)-1-ethyl-4-phenyl-1H-imidazo[4,5-c]pyridin-7-yl]carbonyl}-3-piperidinecarboxamide;
(2-aminoethyl)(2-{[2-(4-amino-1,2,5-oxadiazol-3-yl)-1-ethyl-4-phenyl-1H-imidazo[4,5-c]pyridin-7-yl]oxy}ethyl)amine;
4-(1-ethyl-4-phenyl-7-{[2-(1-piperazinyl)ethyl]oxy}-1H-imidazo[4,5-c]pyridin-2-yl)-1,2,5-oxadiazol-3-amine;
4-(7-{[2-(4-acetyl-1-piperazinyl)ethyl]oxy}-1-ethyl-4-phenyl-1H-imidazo[4,5-c]pyridin-2-yl)-1,2,5-oxadiazol-3-amine trifluoroacetate;
4-(1-ethyl-7-{[3-(4-methyl-1-piperazinyl)propyl]oxy}-4-phenyl-1H-imidazo[4,5-c]pyridin-2-yl)-1,2,5-oxadiazol-3-amine;
4-(1-ethyl-4-phenyl-7-{[3-(1-piperazinyl)propyl]oxy}-1H-imidazo[4,5-c]pyridin-2-yl)-1,2,5-oxadiazol-3-amine;
4-(1-ethyl-4-phenyl-7-{[2-(1-piperidinyl)ethyl]oxy}-1H-imidazo[4,5-c]pyridin-2-yl)-1,2,5-oxadiazol-3-amine trifluoroacetate;
(3-{[2-(4-amino-1,2,5-oxadiazol-3-yl)-1-ethyl-4-phenyl-1H-imidazo[4,5-c]pyridin-7-yl]oxy}propyl)[2-(dimethylamino)ethyl]methylamine;
3-[(3-{[2-(4-amino-1,2,5-oxadiazol-3-yl)-1-ethyl-4-phenyl-1H-imidazo[4,5-c]pyridin-7-yl]oxy}propyl)amino]-1,2-propanediol;
N-(3-amino-2-hydroxypropyl)-2-(4-amino-1,2,5-oxadiazol-3-yl)-1-ethyl-4-phenyl-1H-imidazo[4,5-c]pyridine-7-carboxamide;
N-(2-amino-3-hydroxypropyl)-2-(4-amino-1,2,5-oxadiazol-3-yl)-1-ethyl-4-phenyl-1H-imidazo[4,5-c]pyridine-7-carboxamide;
N-(3-{2-(4-amino-1,2,5-oxadiazol-3-yl)-7-[(3-aminopropyl)oxy]-1-ethyl-1H-imidazo[4,5-c]pyridin-4-yl}phenyl)-N′-phenylurea;
3-{[2-(4-amino-1,2,5-oxadiazol-3-yl)-1-ethyl-4-phenyl-1H-imidazo[4,5-c]pyridin-7-yl]oxy}-1-propanol;
(4-{[2-(4-amino-1,2,5-oxadiazol-3-yl)-1-ethyl-4-phenyl-1H-imidazo[4,5-c]pyridin-7-yl]carbonyl}-2-piperazinyl)methanol;
4-[1-ethyl-7-({3-[(methyloxy)methyl]-1-piperazinyl}carbonyl)-4-phenyl-1H-imidazo[4,5-c]pyridin-2-yl]-1,2,5-oxadiazol-3-amine;
4-(7-{[3-({[2,4-bis(methyloxy)phenyl]methyl}amino)propyl]oxy}-1-ethyl-4-phenyl-1H-imidazo[4,5-c]pyridin-2-yl)-1,2,5-oxadiazol-3-amine;
(2S)-2-[(3-{[2-(4-amino-1,2,5-oxadiazol-3-yl)-1-ethyl-4-phenyl-1H-imidazo[4,5-c]pyridin-7-yl]oxy}propyl)amino]-4-methyl-1-pentanol;
diethyl 1-[2-(4-amino-1,2,5-oxadiazol-3-yl)-1-ethyl-7-hydroxy-4-phenyl-1H-imidazo[4,5-c]pyridin-6-yl]-1,2-hydrazinedicarboxylate; and
4-(2-(4-amino-1,2,5-oxadiazol-3-yl)-1-ethyl-7-{[3-({2-[4-(methyloxy)phenyl]ethyl}amino)propyl]oxy}-1H-imidazo[4,5-c]pyridin-4-yl)-2-methyl-3-butyn-2-ol.

24. A pharmaceutically acceptable salt, hydrate, solvate or pro-drug of a compound of claim 23.

25. A pharmaceutical composition comprising a compound according to claim 1, and/or a pharmaceutically acceptable salt, hydrate, solvate or pro-drug thereof and a pharmaceutically acceptable carrier.

26. A process for preparing a pharmaceutical composition containing a pharmaceutically acceptable carrier or diluent and an effective amount of a compound of Formula (I) as described in claim 1 and/or a pharmaceutically acceptable salt, hydrate, solvate or pro-drug thereof, which process comprises bringing the compound of Formula (I) and/or a pharmaceutically acceptable salt, hydrate, solvate or pro-drug thereof into association with a pharmaceutically acceptable carrier or diluent.

27. A method of treating or lessening the severity of a disease or condition selected from cancer and arthritis in a mammal in need thereof, which comprises administering to such mammal a therapeutically effective amount of a compound of Formula I, as described in claim 1 and/or a pharmaceutically acceptable salt, hydrate, solvate or pro-drug thereof.

28. The method of claim 27 wherein the mammal is a human.

29. A method of treating or lessening the severity of a disease or condition selected from cancer and arthritis in a mammal in need thereof, which comprises administering to such mammal a therapeutically effective amount of a compound of Formula II, as described in claim 3 and/or a pharmaceutically acceptable salt, hydrate, solvate or pro-drug thereof.

30. The method of claim 29 wherein the mammal is a human.

31. The method according to claim 27 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

32. The method according to claim 29 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

33. Use of a compound of Formula (I), as described in claim 1 and/or a pharmaceutically acceptable salt, hydrate, solvate or pro-drug thereof, in the manufacture of a medicament for use in treating or lessening the severity of a disease or condition selected from cancer and arthritis.

34. The method of inhibiting Akt activity in a mammal in need thereof, which comprises administering to such mammal a therapeutically effective amount of a compound of Formula I, as described in claim 1 and/or a pharmaceutically acceptable salt, hydrate, solvate or pro-drug thereof.

35. The method of claim 34 wherein the mammal is a human.

36. A method of treating cancer in a mammal in need thereof, which comprises: administering to such mammal a therapeutically effective amount of
a) a compound of Formula (I), as described in claim 1 and/or a pharmaceutically acceptable salt, hydrate, solvate or pro-drug thereof; and
b) at least one anti-neoplastic agent.

37. The method claim 36, wherein the at least one anti-neoplastic agent is selected from the group consisting essentially of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; non-receptor tyrosine kinase angiogenesis inhibitors; immunotherapeutic agents;
proapoptotic agents; and cell cycle signaling inhibitors.

38. The method of claim 36, wherein the at least one anti-neoplastic agent is an anti-microtubule agent selected from diterpenoids and vinca alkaloids.

39. The method of claim 36, wherein the at least one anti-neoplastic agent is a diterpenoid.

40. The method of claim 36, wherein the at least one anti-neoplastic agent is a vinca alkaloid.

41. The method of claim 36, wherein the at least one anti-neoplastic agent is a platinum coordination complex.

42. The method of claim 36, wherein the at least one anti-neoplastic agent is paclitaxel, carboplatin, or vinorelbine.

43. The method of claim 36, wherein the at least one anti-neoplastic agent is paclitaxel.

44. The method of claim 36, wherein the at least one anti-neoplastic agent is carboplatin.

45. The method of claim 36, wherein the at least one anti-neoplastic agent is vinorelbine.

46. The method of claim 36, wherein the at least one anti-neoplatic agent is a signal transduction pathway inhibitor.

47. The method of claim 46, wherein the signal transduction pathway inhibitor is an inhibitor of a growth factor receptor kinase selected from the group consisting of VEGFR2, TIE2, PDGFR, BTK, IGFR-1, TrkA, TrkB, TrkC, and c-fms.

48. The method of claim 46, wherein the signal transduction pathway inhibitor is an inhibitor of a serine/threonine kinase selected from the group consisting of rafk, akt, and PKC-zeta.

49. The method of claim 46, wherein the signal transduction pathway inhibitor is an inhibitor of a serine/threonine kinase selected from the src family of kinases.

50. The method of claim 49, wherein the signal transduction pathway inhibitor is an inhibitor of c-src.

51. The method of claim 46, wherein the signal transduction pathway inhibitor is an inhibitor of Ras oncogene selected from inhibitors of farnesyl transferase and geranylgeranyl transferase.

52. The method of claim 46, wherein the signal transduction pathway inhibitor is an inhibitor of a serine/threonine kinase selected from the group consisting of PI3K.

53. The method of claim 36, wherein the at least one anti-neoplastic agent is a cell cycle signaling inhibitor.

54. The method of claim 53, wherein the cell cycle signaling inhibitor is selected from inhibitors of the group CDK2, CDK4, and CDK6.

55. A pharmaceutical combination as claimed in claim 36 for use in therapy.

56. The use of a pharmaceutical combination as claimed in claim 36 for the preparation of a medicament useful in the treatment of cancer.

57. A compound selected from:
4-(7-Bromo-4-chloro-1-ethyl-1H-imidazo[4,5-c]pyridin-2-yl)-1,2,5-oxadiazol-3-amine;
2-(4-Amino-1,2,5-oxadiazol-3-yl)-1-methyl-4-phenyl-1H-imidazo[4,5-c]pyridine-7-carboxylic acid; and
Ethyl 4-chloro-5-nitro-6-phenyl-3-pyridinecarboxylate.

58. A process for preparing a compound of Formula (I), and/or pharmaceutically acceptable salts, hydrates, solvates and pro-drugs thereof, which comprises converting ethyl 4-chloro-5-nitro-6-phenyl-3-pyridinecarboxylate into a compound of Formula (I), and thereafter optionally preparing a pharmaceutically acceptable salt, hydrate, solvate or pro-drug thereof.

59. A process for preparing a compound of Formula (II), and/or pharmaceutically acceptable salts, hydrates, solvates and pro-drugs thereof, which comprises converting 4-(7-Bromo-4-chloro-1-ethyl-1H-imidazo[4,5-c]pyridin-2-yl)-1,2,5-oxadiazol-3-amine into a compound of Formula (II), and thereafter optionally preparing a pharmaceutically acceptable salt, hydrate, solvate or pro-drug thereof.

60. A process for preparing a compound of Formula (II), and/or pharmaceutically acceptable salts, hydrates, solvates and pro-drugs thereof, which comprises converting 2-(4-Amino-1,2,5-oxadiazol-3-yl)-1-methyl-4-phenyl-1H-imidazo[4,5-c]pyridine-7-carboxylic acid into a compound of Formula (II), and thereafter optionally preparing a pharmaceutically acceptable salt, hydrate, solvate or pro-drug thereof.

61. A method of treating or lessening the severity of a disease or condition selected from cancer and arthritis in a mammal in need thereof, which comprises administering to such mammal a therapeutically effective amount of 4-[1-Ethyl-7-(piperidin-4-yloxy)-1H-imidazo[4,5-c]pyridin-2-yl]-furazan-3-ylamine and/or a pharmaceutically acceptable salt, hydrate, solvate or pro-drug thereof.
